(19) United States
(12) Patent Application Publication
(10) Pub. No.: US 2024/0093258 A1
(43) Pub. Date: Mar. 21, 2024

(54) GLYCOSIDE PRODUCT BIOSYNTHESIS AND RECOVERY

(71) Applicant: Manus Bio Inc., Waltham, MA (US)

(72) Inventors: Ajikumar PARAYIL KUMARAN, Lexington, MA (US); Christine Nicole S. SANTOS, Boston, MA (US); Jason DONALD, Waltham, MA (US); Aaron LOVE, Waltham, NH (US); Yiying ZHENG, Waltham, MA (US); Adel GHADERI, Waltham, MA (US); Vineet SHASTRY, Waltham, MA (US); Lu CHEN, Waltham, MA (US); Christopher TOOMEY, Waltham, MA (US); Hannah LYNCH, Waltham, MA (US); Eric NIEMINEN, Waltham, MA (US)

(21) Appl. No.: 18/038,067

(22) PCT Filed: Nov. 24, 2021
(86) PCT No.: PCT/US21/60722

§ 371 Date: May 22, 2023

Related U.S. Application Data
(60) Provisional application No. 63/117,534, filed on Nov. 24, 2020.

(51) Int. Cl. C12P 19/60; C12N 9/10; C12N 9/90

(52) U.S. Cl. CPC C12P 19/60; C12N 9/1051; C12N 9/90; C12Y 204/01017; C12Y 503/01009

(57) ABSTRACT

In various aspects and embodiments, the present disclosure provides methods for making glycosylated products, as well as bacterial cells and uridine diphosphate (UDP)-dependent glycosyltransferase (UGT) enzymes useful for the same. In other aspects and embodiments, the disclosure provides methods for high yield and/or high purity recovery of such glycoside products from microbial cultures or cell free reactions. In various aspects and embodiments, the disclosure provides for whole cell bioconversion processes involving the glycosylation of a desired substrate, and/or the recovery of the glycosylated product at high yield and/or high purity.

### BACKGROUND

Glycosyltransferases of small molecules are encoded by a large multigene family in the plant kingdom. These enzymes transfer sugars from nucleotide sugars to a wide range of secondary metabolites, thereby altering the physical and chemical properties of the acceptor molecule. For example, steviol glycosides are a class of compounds found in the leaves of Stevia rebaudiana Bertoni, a perennial shrub of the Asteraceae (Compositae) family native to certain regions of South America. They are characterized structurally by the core terpenoid, steviol, differing by the presence of carbohydrate residues at positions C13 and C19. They accumulate in stevia leaves, composing approximately 10% to 20% of the total dry weight. On a dry weight basis, the four major glycosides found in the leaves of Stevia typically include stevioside, rebaudioside A, rebaudioside C, and dulcoside A. Other steviol glycosides are present at small or trace amounts, including rebaudioside B, D, E, F, G, H, I, J, K, L, M and O, dulcoside B, steviolbioside and rubusoside.

The minor glycosylation product rebaudioside M (RebM) is estimated to be about 200-350 times more potent than sucrose, and is described as possessing a clean, sweet taste with a slightly bitter or licorice aftertaste. Prakash I. et al., Development of Next Generation Stevia Sweetener: Rebaudioside M, Foods 3(1), 162-175 (2014). While RebM is of great interest to the global food industry, its low prevalence in stevia extract necessitates innovative processes for its synthesis.

As another example, mogrosides are triterpene-derived specialized secondary metabolites found in the fruit of the Cucurbitaceae family plant Siraitia grosvenorii (a/k/a monkfruit or Luo Han Guo). Their biosynthesis in fruit involves a number of consecutive glycosylations of the aglycone mogrol. The food industry is increasing its use of mogroside fruit extract as a natural non-sugar food sweetener. For example, mogroside V (mog. V) has a sweetening capacity that is ˜250 times that of sucrose (Kasai et al., Agric Biol Chem (1989)). Moreover, additional health benefits of mogrosides have been identified (Li et al., Chin J Nat Med (2014)).

Purified Mog. V has been approved as a high-intensity sweetening agent in Japan and the extract has gained GRAS status in the USA as a non-nutritive sweetener and flavor enhancer. Extraction of mogrosides from the fruit can yield a product of varying degrees of purity, often accompanied by undesirable aftertaste. In addition, yields of mogroside from cultivated fruit are limited due to low plant yields and particular cultivation requirements of the plant. Mogrosides are present at about 1% in the fresh fruit and about 4% in the dried fruit. Mog. V is the main component, with a content of 0.5% to 1.4% in the dried fruit. Moreover, purification difficulties limit purity for Mog. V, with commercial products from plant extracts being standardized to about 50% Mog. V. It is likely that a pure Mog. V product will achieve greater commercial success than the blend, since it is less likely to have off flavors, will be easier to formulate into products, and has good solubility potential. It is therefore advantageous to produce sweet mogroside compounds via biotechnological processes.

There remains a need for economical methods for producing high value glycosides, including those that are minor products of natural plant extract.

### SUMMARY

In various aspects and embodiments, the present disclosure provides methods for making glycosylated products, as well as bacterial cells and uridine diphosphate (UDP)-dependent glycosyltransferase (UGT) enzymes useful for the same. In other aspects and embodiments, the disclosure provides methods for high yield and/or high purity recovery of glycoside products from microbial cultures or cell free reactions. In various aspects and embodiments, the disclosure provides for whole cell bioconversion processes involving the glycosylation of a desired substrate, followed by recovery of the glycosylated product at high yield and/or high purity.

In one aspect, the invention provides a bacterial cell and method for making a glycosylated product. In particular, the disclosure provides a bacterial cell expressing one or more UGT enzymes for glycosylating a desired substrate according to a whole cell bioconversion process. In some embodiments, the bacterial cell expresses one or more recombinant sucrose synthase enzymes. Sucrose synthase expression can dramatically enhance whole cell glycosylation of fed substrates. Alternatively or in addition, the bacterial cell comprises one or more genetic modifications that increase availability of UDP-sugar. The bacterial cell is cultured in the presence of the substrate for glycosylation, and the glycosylated product is recovered, optionally using a recovery process described herein.

Whole cell bioconversion systems have advantages over cell-free systems, since the cell provides UDP-glucose cofactor regeneration. In embodiments of the present invention, catalysis (glycosylation) is carried out with live bacterial cells, and UDP-glucose cofactor recycling takes place using the cellular metabolism without requiring enzyme feeding or feeding expensive substrates for UDP-glucose regeneration. Various bacterial species may be used in accordance with this disclosure, including E. coli.

In some embodiments, the bacterial cell expresses a recombinant sucrose synthase enzyme, and the bacterial cell may be cultured in the presence of sucrose. In some embodiments, the sucrose synthase enzyme comprises an amino acid sequence that has at least about 70% sequence identity with an amino acid sequence selected from SEQ ID NOS: 1 to 12.

In some embodiments, the microbial cell has one or more genetic modifications that increase UDP-glucose availability, such as a deletion, inactivation, or reduced activity or expression of a gene encoding an enzyme that consumes UDP-glucose. Other UDP-glucose sinks that can be reduced or eliminated include eliminating or reducing activity or expression of genes responsible for lipid glycosylation and LPS biosynthesis, and genes responsible for glycosylating undecaprenyl-diphosphate (UPP). In these or other embodiments, the bacterial cell has a deletion, inactivation, or reduced activity or expression of a gene encoding an enzyme that consumes a precursor to UDP-glucose. In these or other embodiments, the cell has an overexpression or increased activity of one or more genes encoding an enzyme involved in converting glucose-6-phosphate to UDP-glucose. Alternatively or in addition, the bacterial cell has one or more genetic modifications that increase glucose transport. Alternatively or in addition, the microbial cell has one or more genetic modifications that increase UTP production and recycling. Alternatively or in addition, the microbial cell has one or more genetic modifications that increase UDP production. Alternatively or in addition, the microbial cell may have one or more genetic modifications to remove or reduce regulation of glucose uptake. Alternatively or in addition, the microbial cell may have one or more genetic modifications that reduce dephosphorylation of glucose-1-phosphate. Alternatively or in addition, the bacterial cell has one or more genetic modifications that reduce conversion of glucose-1-phosphate to TDP-glucose. Alternatively or in addition, the bacterial cell may have one or more genetic modifications that reduce conversion of glucose-1-phosphate to ADP-glucose.

In various embodiments, the substrates for glycosylation are provided as a plant extract or fraction thereof, or are produced synthetically or by a biosynthesis process. Exemplary substrates include various secondary metabolites, such as those selected from terpenoids or terpenoid glycosides, flavonoids or flavonoid glycosides, cannabinoids or cannabinoid glycosides, polyketides or polyketide glycosides, stilbenoids or stilbenoid glycosides, and polyphenols or polyphenol glycosides. Plant extracts can be fractionated or otherwise enriched for desired substrates. In some embodiments, the substrates comprise terpenoid glycosides, such as steviol or steviol glycosides, or mogrol or mogrol glycosides. UGT enzymes, as well as the relevant substrates (including as fractions enriched for desired substrates) can be selected to produce the desired glycosylated product. In some embodiments, the glycosylated product comprises one or more steviol glycosides, such as RebM, RebE, RebD, RebB, and/or RebI, or mogrol glycosides such as mog. IV, mog. IVA, mog. V, mog. VI, isomog. V, and/or siamenoside, among others.

In other aspects and embodiments, the invention provides an engineered UDP-dependent glycosyltransferase (UGT) enzyme with high productivity for glycosylating substrates, including terpenoid glycoside substrates, and including in connection with the bacterial cells and methods described herein. In some embodiments, the engineered UGT enzyme comprises an amino acid sequence that has at least about 70% sequence identity to SEQ ID NO: 13, and having one or more amino acid modifications that improve glycosylating activity on terpenoid glycoside substrates (e.g., steviol glycoside substrates). In still other embodiments, the UGT enzyme comprises an amino acid sequence that has at least about 70% sequence identity to SEQ ID NO: 14, and having one or more amino acid modifications that improve glycosylating activity on terpenoid glycoside substrates (e.g., steviol glycoside substrates). In still other embodiments, the UGT enzyme comprises an amino acid sequence that has at least about 70% sequence identity to SEQ ID NO: 15, and having one or more amino acid substitutions that improve glycosylating activity on terpenoid glycoside substrates (e.g., steviol glycoside substrates).

In other aspects and embodiments, the invention provides UGT enzymes (including microbial cells expressing the same) for glycosylating a mogrol or mogrol glycoside substrate. In these aspects and embodiments, the method comprises contacting the substrate with a UGT enzyme in the presence of UDP-sugar. The UGT enzyme may comprise an amino acid sequence that has at least about 80% sequence identity to an amino acid sequence selected from: SEQ ID NO: 84, SEQ ID NO: 80, SEQ ID NO: 46, SEQ ID NO: 83, SEQ ID NO: 82, SEQ ID NO: 73, SEQ ID NO: 72, SEQ ID NO: 78, SEQ ID NO: 54, SEQ ID NO: 74, SEQ ID NO: 75, SEQ ID NO: 76, SEQ ID NO: 13, SEQ ID NO: 14, SEQ ID NO: 15, SEQ ID NO: 16, SEQ ID NO: 29, and SEQ ID NO: 79. In these embodiments, the mogrol or mogrol glycoside substrate may be provided as a plant extract or fraction thereof, such as a monkfruit extract or fraction thereof. For example, the substrate may comprise (or be enriched for) one or more substrates selected from mogrol, mog. I-A, mog. I-E, mog. II-A, mog. II-E, mog III, mog IVA, mog. IV, and siamenoside. In some embodiments, the glycosylated product may comprise one or more of mog. IV, mog. IVA, mog. V, mog VI, isomog. V, and siamenoside. In various embodiments, the UGT enzymes may be capable of primary glycosylation at the C3 and C24 hydroxyl of a mogrol core, and 1-2 and 1-6 branching glycosylations of the C3 and/or C24 primary glycosyl groups.

In some embodiments with regard to producing mogrol glycosides, the substrates are cultured with a microbial cell expressing the UGT enzymes. Exemplary microbial cells include bacterial cells engineered for whole cell bioconversion processes as described herein. In still other embodiments, the microbial cell is a yeast cell. However, in still other embodiments, the substrates are incubated with a cell lysate comprising the UGT enzymes, or are incubated with purified recombinant UGT enzymes according to known techniques.

In some aspects, the invention provides a method for producing and recovering a glycoside product. In such embodiments, the method comprises converting a substrate for glycosylation to a target glycoside product by enzymatic transfer of one or more sugar moieties in a cell-free reaction or in a microbial culture, which may optionally employ a method, UGT enzyme, and/or microbial strain described herein. The method further comprises recovering the glycoside products from the reaction or culture, where the recovering comprises one or more of: adjusting the pH of the reaction or culture to below about pH 5 or above about 10, raising the temperature to at least about 50° C., and adding one or more glycoside solubilizers; followed by enzyme or biomass removal.

Conventionally, biomass removal is the first step in recovery, to remove large cellular debris, and to avoid disruption of cells that would complicate downstream purification. However, in accordance with embodiments of the present invention, the culture material can be highly viscous and difficult to process. By treating the culture material as described herein, prior to biomass or enzyme removal, it is possible to produce a product with desirable qualities, including: high purity of glycoside product, attractive color, easy solubilization, odorless, and/or high recovery yield. For example, initial pH and temperature adjustment of the culture can change fluid characteristics of the broth, and increase efficiency of a disc stack separator for biomass removal. Further, solubility and therefore yield of glycoside product can be substantially increased by the pH and/or temperature adjustment, and/or addition of a glycoside solubilizer.

Other aspects and embodiments of the disclosure will be apparent from the following detailed disclosure and examples.

### BRIEF DESCRIPTION OF THE DRAWINGS

FIG. 1 shows improvement of steviol glycoside bioconversion from two chromosomal modifications ((1) ΔotsA-otsB; (2) ΔotsA-otsB, insertion of ugpA) of engineered E. coli cells expressing UGT enzymes. Fold improvement is with respect to total steviol glycoside conversion.

FIG. 2 shows improvement of steviol glycoside bioconversion from overexpressed genes in engineered E. coli cells expressing UGT enzymes. Genes were complemented on plasmid. Fold improvement is with respect to total steviol glycoside conversion.

Complemented genes are, from left to right: (1) control (empty plasmid), (2) pgm (SEQ ID NO: 92) and galU (SEQ ID NO: 93), (3) pgm (SEQ ID NO: 92), (4) galU (SEQ ID NO: 93), (5) ugpA (SEQ ID NO: 95), (6) ycjU (SEQ ID NO: 94), (7) adk (SEQ ID NO: 96), (8) ndk (SEQ ID NO: 97), (9) pyrH, (10) cmk (SEQ ID NO: 98).

FIG. 3 shows improvement of steviol glycoside bioconversion by engineered E. coli cells expressing UGT enzymes, and with overexpressed sucrose synthases. Genes were complemented on plasmid. Fold improvement is with respect to total steviol glycoside conversion. Complemented genes are, from left to right: (1) control (empty plasmid), (2) StSus1 (SEQ ID NO: 1), (3) StSus2 (SEQ ID NO: 2), (4) StSus2_S11E (SEQ ID NO: 3), (5) AcSuSy (SEQ ID NO: 4), (6) AcSuSy_L637M-T640V (SEQ ID NO: 5), (7) AtSus1 (SEQ ID NO: 6), (8) AtSus3 (SEQ ID NO: 7), (9) VrSS1 (SEQ ID NO: 8), (10) VrSS1_S11E (SEQ ID NO: 9), (11) GmSS (SEQ ID NO: 10), (12) GmSS_S11E (SEQ ID NO: 11), (13) AtSusA (SEQ ID NO: 12).

FIG. 4 shows improvement of steviol glycoside bioconversion by engineered E. coli cells expressing UGT enzymes, and with various gene knockouts. Fold improvement is with respect to total steviol glycoside conversion. Deletions are (from left to right): (1) ΔotsA, (2) Δugd, (3) ΔrfaQPSBU, (4) ΔyfdGHI, (5) ΔwcaJ, and (6) ΔglgC.

FIG. 5 shows improvement of steviol glycoside bioconversion by engineered E. coli cells expressing the UGT enzyme defined by SEQ ID NO: 14 (MbUGT1,2.3). Fold improvement is with respect to % steviol glycoside conversion of the parent UGT enzyme (SEQ ID NO: 13).

FIG. 6 shows improvement of steviol glycoside bioconversion by engineered E. coli cells expressing the UGT enzyme defined by SEQ ID NO: 15 (MbUGT1,2.4). Fold improvement is with respect to % steviol glycoside conversion of the parent UGT enzyme (SEQ ID NO: 14).

FIG. 7 shows improvement of steviol glycoside bioconversion by engineered E. coli cells expressing the UGT enzyme defined by SEQ ID NO: 16 (MbUGT1,2.5). Fold improvement is with respect to % steviol glycoside conversion of the parent UGT enzyme (SEQ ID NO: 15).

FIG. 8 shows bioconversion by an engineered E. coli bioconversion strain of stevia leaf extract to a mix of rebaudioside E and rebaudioside D, by expression of the UGT enzyme of SEQ ID NO: 15 (MbUGT1,2.4), and to rebaudioside I by expression of the UGT enzyme of SEQ ID NO: 25 (MbUGT1-3.3).

FIG. 9 shows bioconversion by an engineered E. coli bioconversion strain of stevia leaf extract to a mix of rebaudioside B and steviolbioside, by expression of the UGT enzymes of SEQ ID NO: 31 and SEQ ID NO: 99.

FIG. 10 is a flow diagram illustrating a conventional process for recovery of steviol glycosides. Typically, biomass removal is conducted first to remove large cellular debris, enzymes, and whole intact cells to facilitate purification of the desired product.

FIG. 11 is a flow diagram illustrating an exemplary process for glycoside product recovery in accordance with embodiments of the present invention. pH and/or temperature adjustment and/or the addition of solubilizing agents are employed before biomass removal, to improve the physical properties of the culture material for processing, which in turn facilitates biomass removal while increasing yield of glycoside products.

FIG. 12 shows the effect of various treatments on the separation of biomass from aqueous broth following centrifugation. The compactness of the pellet and clarity of supernatant serve as indicators of the ease of biomass removal. Tubes from left to right: (1) 22° C. (room temperature), pH 6.64; (2) 22° C. (room temperature), ethanol; (3) 22° C. (room temperature), pH 3.78, +ethanol; (4) 22° C. (room temperature), pH 3.78; (5) 70° C., pH 6.64; (6) 70° C., ethanol; (7) 70° C., pH 3.78, +ethanol; and (8) 70° C., pH 3.78.

FIG. 13 shows that filtration of the solution prior to recrystallization impacts purity, and the selection of filter material has a significant impact on the quality of the final product. FIG. 13 compares the use of polypropylene (PP) (left) and polyethersulfone (PES) (right) material to filter the solution prior to recrystallization. Highly pure RebM final product (>98%) is dissolved in propylene glycol to a concentration of 10 wt %. The use of a PP filter results in a solution that is very cloudy whereas the use of PES filter yields a solution that is clear.

FIG. 14 shows the solubility (bottom curve) and metastable limit curve (top curve), defining the metastable zone width, as determined for RebM in water, allowing for control of crystal growth.

FIG. 15A, 15B show the solubility (bottom curve) and metastable limit curve (top curve), defining the metastable zone width, as determined for RebM in 67% water/33% ethanol at pH 7, allowing for control of crystal growth in this solvent system. FIG. 15A is 0% glycerol, while FIG. 15B includes 0.5% glycerol.

FIG. 16A, 16B show the solubility (bottom curve) and metastable limit curve (top curve), defining the metastable zone width, as determined for RebM in 67% water/33% ethanol at pH 11, allowing for control of crystal growth in this solvent system. FIG. 16A is 0% glycerol, while FIG. 16B includes 0.5% glycerol.

FIG. 17A shows the bioconversion of mogrol into mogroside compounds (mog IIE, mog-IE, and mog-IA) using engineered E. coli strains expressing either Enzyme 1 (SEQ ID NO: 71) or Enzyme 2 (SEQ ID NO: 33). FIG. 17B shows the bioconversion of mogrol into mogroside-IA using engineered E. coli strains expressing Enzyme 1 (SEQ ID NO: 71), Enzyme 3 (SEQ ID NO: 81), Enzyme 4 (SEQ ID NO: 82), and Enzyme 5 (SEQ ID NO: 83).

FIG. 18A and FIG. 18B show the bioconversion of mog-IA (FIG. 18A) or mog-IE (FIG. 18B) into mog-IIE using engineered E. coli strains expressing either Enzyme 1 (SEQ ID NO: 84), Enzyme 2 (SEQ ID NO: 71), or Enzyme 3 (SEQ ID NO: 33).

FIG. 19 shows the production of Mog-III or siamenoside from Mog II-E by engineered E. coli strains expressing Enzyme 1 (SEQ ID NO: 72), Enzyme 2 (SEQ ID NO: 54), or Enzyme 3 (SEQ ID NO: 13).

FIG. 20 shows the in vitro production of MogII-A2 by E. coli cells expressing Enzyme 1 (SEQ ID NO: 73).

FIG. 21 shows glycosylation products produced by action of UGT enzymes on steviol and steviol glycoside intermediates.

FIG. 22 illustrates glycosylation routes to Mog. V. Bubble structures represent different mogrosides. White tetra-cyclic core represents mogrol. The numbers below each structure indicate the particular glycosylated mogroside. Black circles represent C3 or C24 glucosylations. Dark grey vertical circles represent 1,6-glucosylations. Light grey horizontal circles represent 1,2-glucosylations. Abbreviations: Mog, mogrol; sia, siamenoside.

### DETAILED DESCRIPTION

In various aspects and embodiments, the present disclosure provides methods for making glycosylated products, as well as bacterial cells and uridine diphosphate (UDP)-dependent glycosyltransferase (UGT) enzymes useful for the same. In other aspects and embodiments, the disclosure provides methods for high yield and/or high purity recovery of glycoside products from microbial cultures or cell free reactions. In various aspects and embodiments, the disclosure provides for whole cell bioconversion processes involving the glycosylation of a desired substrate, followed by recovery of the glycosylated product at high yield and/or high purity.

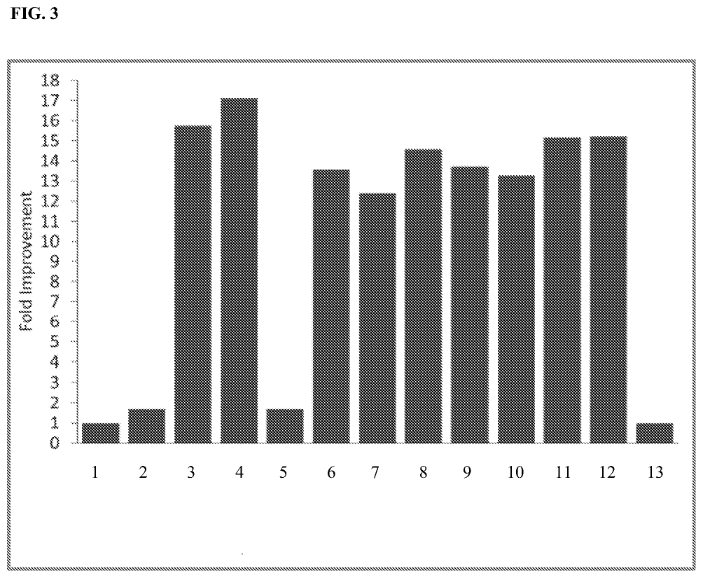

In one aspect, the invention provides a bacterial cell and method for making a glycosylated product. The bacterial cell expresses one or more UGT enzymes for glycosylating a desired substrate. In some embodiments, the bacterial cell further expresses one or more recombinant sucrose synthase enzymes. Sucrose synthase expression can dramatically enhance whole cell glycosylation of fed substrates (see FIG. 3). Alternatively or in addition, the bacterial cell comprises one or more genetic modifications that increase availability of UDP-sugar. The bacterial cell is cultured in the presence of the substrate for glycosylation, and the glycosylated product is recovered, optionally using a recovery process described herein.

Whole cell bioconversion systems have advantages over cell-free systems for glycosylation reactions, since the cell provides UDP-glucose cofactor regeneration. This is in contrast to processes that use enzymes from cell lysis or secretion outside the cell, which requires an exogenous UDP-glucose supply or UDP-glucose precursor or UDP-glucose regeneration mechanism or UDP-glucose regeneration enzyme system. In embodiments of the present invention, catalysis (glycosylation) is carried out with live bacterial cells, and UDP-glucose cofactor recycling takes place using the cellular metabolism without requiring enzyme feeding or the feeding of expensive substrates for UDP-glucose regeneration. Various bacterial species may be used in accordance with this disclosure, including species of Escherichia, Bacillus, Rhodobacter, Zymomonas, or Pseudomonas. In some embodiments, the bacterial cell is Escherichia coli, Bacillus subtilis, Rhodobacter capsulatus, Rhodobacter sphaeroides, Zymomonas mobilis, or Pseudomonas putida. In exemplary embodiments, the bacterial cell is E. coli.

In some embodiments, the bacterial cell expresses a recombinant sucrose synthase enzyme. In some embodiments, the bacterial cell expressing a sucrose synthase enzyme is cultured in the presence of sucrose. In some embodiments, the sucrose synthase enzyme comprises an amino acid sequence that has at least about 70% sequence identity with an amino acid sequence selected from SEQ ID NOS: 1 to 12. As demonstrated in FIG. 3, expression of a sucrose synthase in the bacterial cell provides for dramatic enhancement of glycosylation of substrates fed to whole cells. In various embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with an amino acid sequence selected from SEQ ID NOS: 1 to 12.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 2. In some embodiments, the sucrose synthase enzyme comprises an S11E or S11D substitution with respect to the amino acid sequence of SEQ ID NO: 2.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 3. In some embodiments, the sucrose synthase enzyme comprises amino acid substitutions at one or more of L637 (e.g., (L637M) and T640 (e.g., T640V, T640L, T640L, or T640A), with respect to the amino acid sequence of SEQ ID NO: 3.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 5.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 6.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 7. In some embodiments, the sucrose synthase enzyme comprises an S11E or S11D substitution with respect to the amino acid sequence of SEQ ID NO: 7.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 8. In some embodiments, the sucrose synthase enzyme comprises an S11E or S11D substitution with respect to the amino acid sequence of SEQ ID NO: 8.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 9.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 10. In some embodiments, the sucrose synthase enzyme comprises an S11E or S11D substitution with respect to the amino acid sequence of SEQ ID NO: 10.

In some embodiments, the sucrose synthase enzyme comprises an amino acid sequence that has at least about 80%, or at least about 85%, or at least about 90%, or at least about 95%, or at least about 97%, or at least about 98%, or at least about 99% sequence identity with SEQ ID NO: 11.

Knowledge of the three-dimensional structure of an enzyme and the location of relevant active sites, substrate-binding sites, and other interaction sites can facilitate the rational design of derivatives and provide mechanistic insight into the phenotype of specific changes. Plant sucrose synthase enzymes have shown increased activity when the highly conserved S11 and analogous positions are phosphorylated. In some embodiments, the sucrose synthase enzyme comprises an S11E or S11D mutation, which mimics phosphorylation by placing a negative charge where the negatively charged phosphate would be found. Other modifications to the sucrose synthase enzyme can be guided by publicly available structures, such as those described or referenced in Stein O. and Granot D., An Overview of Sucrose Synthases in Plants, Front Plant Sci. 2019; 10: 95.

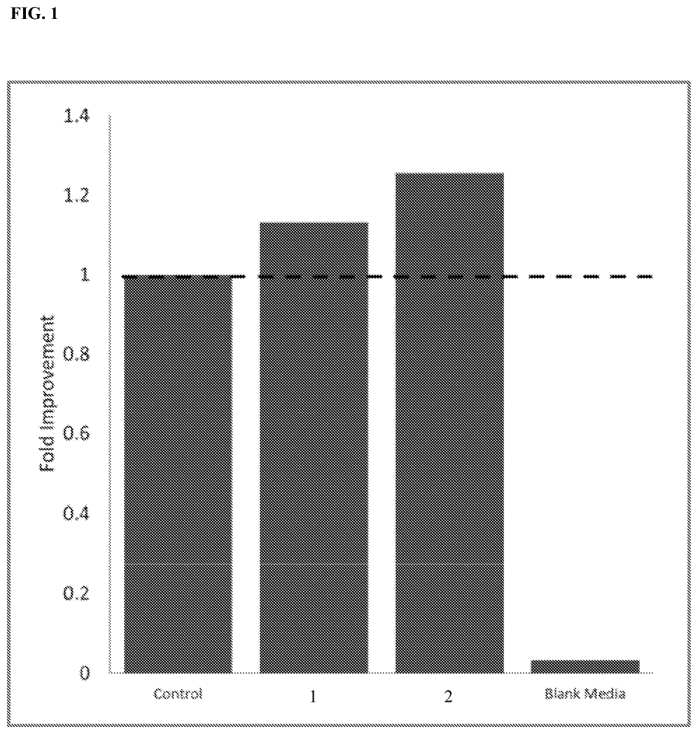

Alternatively or in addition, the bacterial cell comprises one or more genetic modifications that improve the availability of UDP-sugar (e.g., UDP-glucose), which as shown in FIGS. 1, 2, and 4, enhance whole cell bioconversion for glycosylating a desired substrate. Wild-type UDP-glucose levels in exponentially growing E. coli is about 2.5 mM (Bennett B D, Kimball E H, Gao M, Osterhout R, Van dien S J, Rabinowitz J D. Absolute metabolite concentrations and implied enzyme active site occupaney in Escherichia coli. Nat Chem Biol. 2009; 5(8):593-9.). In some embodiments, genetic modifications to the host cell are engineered to increase UDP-glucose, e.g., to at least about 5 mM, or at least about 10 mM, in exponentially growing cells (e.g., that do not have recombinant expression of UGT enzymes).

In some embodiments, the microbial cell has a deletion, inactivation, or reduced activity or expression of a gene encoding an enzyme that consumes UDP-glucose. For example, the bacterial cell may have a deletion, inactivation, or reduced activity or expression of ushA (UDP-sugar hydrolase) and/or one or more of galE, galT, galK, and galM (which are responsible for UDP-galactose biosynthesis from UDP-glucose), or ortholog thereof in the bacterial species. In some embodiments, galETKM genes are inactivated, deleted, or substantially reduced in expression or activity. Alternatively or in addition, the bacterial cell has a deletion, inactivation, or reduced activity or expression of E. coli otsA (trehalose-6-phosphate synthase), or ortholog thereof in the bacterial species. Alternatively or in addition, the microbial cell has a deletion, inactivation, or reduced activity or expression of E. coli ugd (UDP-glucose 6-dehydrogenase), or ortholog thereof in the bacterial species. Reducing or eliminating activity of otsA and ugd can remove or reduce UDP-glucose sinks to trehalose or UDP-glucuronidate, respectively.

Other UDP-glucose sinks that can be reduced or eliminated include eliminating or reducing activity or expression of genes responsible for lipid glycosylation and LPS biosynthesis, and genes responsible for glycosylating undecaprenyl-diphosphate (UPP). Genes involved in glycosylating lipids or LPS biosynthesis include E. coli waaG (lipopolysaccharide glucosyltransferase 1), E. coli waaO (UDP-D-glucose:(glucosyl)LPS α-1,3-glucosyltransferase)), and E. coli waaJ (UDP-glucose:(glycosyl)LPS α-1,2-glucosyltransferase)). Genes responsible for glycosylating undecaprenyl-diphosphate (UPP) include E. coli yfdG (putative bactoprenol-linked glucose translocase), E. coli yfdH (bactoprenol glucosyl transferase), E. coli yfdI (serotype specific glucosyl transferase), and E. coli wcaJ (undecaprenyl-phosphate glucose phosphotransferase). Deletion, inactivation, or reduction in activity or expression of one or more of these gene products (or corresponding orthologs in the bacterial cell) can increase UDP-glucose availability.

In these or other embodiments, the bacterial cell has a deletion, inactivation, or reduced activity or expression of a gene encoding an enzyme that consumes a precursor to UDP-glucose. For example, in some embodiments, the bacterial cell has a deletion, inactivation, or reduced activity or expression of pgi (glucose-6 phosphate isomerase), or ortholog thereof in the bacterial species of the host cell.

In these or other embodiments, the cell has an overexpression or increased activity of one or more genes encoding an enzyme involved in converting glucose-6-phosphate to UDP-glucose. For example, pgm (phosphoglucomutase) and/or galU (UTP-glucose-1-phosphate uridylyltransferase) (or ortholog or derivative thereof) can be overexpressed, or modified to increase enzyme productivity. Alternatively or in addition, E. coli ycjU (β-phosphoglucomutase), which converts glucose-6-phosphate to glucose-1-phosphate, and Bifidobacterium bifidum ugpA, which converts glucose-1-phosphate to UDP-glucose, or ortholog or derivative of these enzymes, can be overexpressed, or modified to increase enzyme productivity.

Alternatively or in addition, the bacterial cell has one or more genetic modifications that increase glucose transport. Such modifications include increased expression or activity of E. coli galP (galactose:H+symporter) and E. coli glk (glucokinase), or alternatively expression of Zymomonas mobilis glf and E. coli glk, or orthologs, or engineered derivatives of these genes.

Alternatively or in addition, the microbial cell has one or more genetic modifications that increase UTP production and recycling. Such modifications include increased expression or activity of, E. coli adk (adenylate kinase), or E. coli ndk (nucleoside diphosphate kinase), or orthologs, or engineered derivatives of these enzymes.

Alternatively or in addition, the microbial cell has one or more genetic modifications that increase UDP production. Such modifications include overexpression or increased activity of one or more of E. coli upp (uracil phosphoribosyltransferase), E. coli dctA (C4 dicarboxylate/orotate:H+symporter), E. coli pyrE (orotate phosphoribosyltransferase), E. coli pyrF (orotidine-5′-phosphate decarboxylase), E. coli pyrH (UMP kinase), and E. coli cmk (cytidylate kinase), including orthologs, or engineered derivatives thereof. For example, in some embodiments, the microbial cell overexpresses or has increased activity of upp, pyrH and cmk, or ortholog or engineered derivative thereof. Alternatively, the microbial cell overexpresses or has increased activity of dctA, pyre, pyrH and cmk, or ortholog or engineered derivative thereof.

Alternatively or in addition, the microbial cell may have one or more genetic modifications to remove or reduce regulation of glucose uptake. For example, the microbial cell may have a deletion, inactivation, or reduced expression of sgrS, which is a small regulatory RNA in E. coli.

Alternatively or in addition, the microbial cell may have one or more genetic modifications that reduce dephosphorylation of glucose-1-phosphate. Exemplary modifications include deletion, inactivation, or reduced expression or activity of one or more of E. coli agp (glucose-1-phosphatase), E. coli yihX (α-D-glucose-1-phosphate phosphatase), E. coli ybiV (sugar phosphatase), E. coli yidA (sugar phosphatase), E. coli yigL (phosphosugar phosphatase), and E. coli phoA (alkaline phosphatase), or an ortholog thereof in the bacterial cell.

Alternatively or in addition, the bacterial cell may have one or more genetic modifications that reduce conversion of glucose-1-phosphate to TDP-glucose. Exemplary modifications include deletion, inactivation, or reduced expression or activity of one or more of E. coli rffH (dTDP-glucose pyrophosphorylase) and E. coli rfbA (dTDP glucose pyrophosphorylase), or an ortholog thereof in the bacterial cell.

Alternatively or in addition, the bacterial cell may have one or more genetic modifications that reduce conversion of glucose-1-phosphate to ADP-glucose. Exemplary modifications include deletion, inactivation, or reduced expression or activity of E. coli glgC (glucose-1-phosphate adenylyltransferase), or an ortholog thereof in the bacterial cell.

For example, in some embodiments, ushA (UDP-sugar diphosphatase) and galETKM or orthologs thereof are deleted, inactivated, or reduced in expression or activity; pgi (glucose-6-phosphate isomerase) or ortholog thereof is deleted, inactivated, or reduced in expression or activity; E. coli pgm (SEQ ID NO: 92) and/or ycjU (SEQ ID NO: 94) or ortholog are overexpressed or a derivative is expressed having increased activity as compared to the wild type enzyme; and E. coli galU (SEQ ID NO: 93) and/or Bifidobacterium bifidum ugpA (SEQ ID NO: 95) or orthologs are overexpressed or derivatives thereof are expressed having increased activity as compared to the wild-type enzyme.

In the various embodiments where the bacterial strain overexpresses E. coli pgm (SEQ ID NO: 92) and/or ycjU (SEQ ID NO: 94) or ortholog, or expresses a derivative having increased activity as compared to the wild-type enzyme; or overexpresses E. coli galU (SEQ ID NO: 93) or expresses Bifidobacterium bifidum ugpA (SEQ ID NO: 95) or orthologs or derivatives thereof (e.g., having higher activity than the wild-type enzyme), complementing genes may comprise amino acid sequences that are at least about 50%, or at least about 60%, or at least about 70%, or at least about 80%, or at least about 90%, or at least about 95%, or at least about 97% identical to SEQ ID NO: 92, SEQ ID NO: 94, SEQ ID NO: 93, or SEQ ID NO: 95, respectively.

For example, in some embodiments, the bacterial cell comprises an overexpression of pgm or an ortholog or derivative thereof (e.g., a derivative having higher activity than the wild-type enzyme), and optionally galU or ortholog or derivative thereof (e.g., a derivative having higher activity than the wild-type enzyme). In still some embodiments, the bacterial cell has a deletion, inactivation, or reduced activity or expression of ushA or ortholog thereof, and/or one or more of galE, galT, galK, and galM, or ortholog(s) thereof. For example, galETKM genes or orthologs thereof may be inactivated, deleted, or reduced in expression or activity. In some embodiments, pgi (glucose-6-phosphate isomerase) or ortholog thereof is deleted, inactivated, or reduced in expression or activity.

Alternatively or in addition, the bacterial cell has a deletion, inactivation, or reduced activity or expression of otsA (trehalose-6-phosphate synthase) or ortholog thereof and/or otsB (trehalose-phosphate phosphatase) or ortholog thereof.

Alternatively or in addition, the bacterial cell has a deletion, inactivation, or reduced activity or expression of one or more of: ugd (UDP-glucose 6-dehydrogenase) or ortholog thereof, rfaQ-G-P-S-B-I-J or ortholog(s) thereof; yfdG-H-I or ortholog(s) thereof, wcaJ or ortholog thereof; and glgC or ortholog thereof.

In exemplary embodiments, the bacterial cell has an overexpression or increased activity or expression of one or more of E. coli ycjU (β-phosphoglucomutase) (SEQ ID NO: 94) or ortholog or derivative thereof, Bifidobacterium bifidum ugpA (UTP-glucose-1-phosphate uridylyltransferase) (SEQ ID NO: 95) or ortholog or derivative thereof, E. coli adk (adenylate kinase) (SEQ ID NO: 96) or ortholog or derivative thereof, E. coli ndk (nucleoside diphosphate kinase) (SEQ ID NO: 97) or ortholog or derivative thereof, and E. coli cmk (cytidine monophosphate kinase) (SEQ ID NO: 98) or ortholog or derivative thereof. In various embodiments, derivative enzymes may be engineered to have higher enzyme activity than the wild-type enzyme. Complementing genes may comprise amino acid sequences that are at least about 50%, or at least about 60%, or at least about 70%, or at least about 80%, or at least about 90%, or at least about 95%, or at least about 97% identical to SEQ ID NO: 94, SEQ ID NO: 95, SEQ ID NO: 96, SEQ ID NO: 97, or SEQ ID NO: 98, respectively.

Other modifications to the bacterial cells to improve UDP-sugar availability are described in US 2020/0087692, which is hereby incorporated by reference in its entirety.

In various embodiments, the substrates for glycosylation are provided as a plant extract or fraction thereof, or are produced synthetically or by a biosynthesis process. Exemplary substrates include various secondary metabolites, such as those selected from terpenoids or terpenoid glycosides, flavonoids or flavonoid glycosides, cannabinoids or cannabinoid glycosides, polyketides or polyketide glycosides, stilbenoids or stilbenoid glycosides, and polyphenols or polyphenol glycosides. Plant extracts can be fractionated or otherwise enriched for desired substrates.

In some embodiments, the substrates comprise terpenoids and/or terpenoid glycosides, such as steviol or steviol glycosides, or mogrol or mogrol glycosides (“mogrosides”). In some embodiments, the substrates have predominantly from 0 to about 4 glycosyl groups, and which may include glucosyl, galactosyl, mannosyl, xylosyl, and/or rhamnosyl groups. In various embodiments, the glycosyl groups are predominately glucosyl. After whole cell bioconversion, in various embodiments the glycosylated product will have at least four, at least five, at least six, or at least seven glycosyl groups (e.g., glucosyl). In various embodiments, whole cell bioconversion involves at least two glycosylation reactions of the substrate by the bacterial cell. In some embodiments, whole cell bioconversion results in a single glycosylation or deglycosylation of the substrate (in the case of a reverse reaction catalyzed by the UGT).

In various embodiments, the substrate is provided as a stevia leaf extract or fraction thereof which may be enriched for target substrates. For example, the stevia leaf extract may comprise or be enriched for one or more of steviol, stevioside, steviolbioside, rebaudioside A, dulcoside A, dulcoside B, rebaudioside C, and rebaudioside F. In some embodiments, at least about 20%, or at least about 30%, or at least about 40%, or at least about 50%, or at least about 75% of the steviol glycosides in the extract or fraction thereof includes one or more of stevioside, steviolbioside, and Rebaudioside A.

UGT enzymes, as well as the relevant substrates (including as plant extract fractions enriched for desired substrates) can be selected to produce the desired glycosylated product. In some embodiments, at least one UGT enzyme comprises an amino acid sequence that has at least about 70% sequence identity to any one of SEQ ID NOS: 13 to 84, and 99. In various embodiments, at least one UGT enzyme comprises an amino acid sequence that has at least about 80%, or at least about 85%, or at least about 90%, or at least about 95%, or at least about 97%, or at least about 99% sequence identity to any one of SEQ ID NOS: 13 to 84, and 99. In accordance with embodiments of this disclosure, UGT enzymes are expressed without secretion or transport signals, and do not contain membrane anchoring domains.

Knowledge of the three-dimensional structure of an enzyme and the location of relevant active sites, substrate-binding sites, and other interaction sites can facilitate the rational design of derivatives and provide mechanistic insight into the phenotype of specific changes. Plant UGTs share a highly conserved secondary and tertiary structure while having relatively low amino acid sequence identity. Osmani et al, Substrate specificity of plant UDP-dependent glycosyltransferases predicted from crystal structures and homology modeling Phytochemistry 70 (2009) 325-347. The sugar acceptor and sugar donor substrates of UGTs are accommodated in a cleft formed between the N- and C-terminal domains. Several regions of the primary sequence contribute to the formation of the substrate binding pocket including structurally conserved domains as well as loop regions differing both with respect to their amino acid sequence and sequence length.

In some embodiments, the substrate is a terpenoid glycoside, and may comprise steviol glycosides or mogrosides in some embodiments. Numerous UGT enzymes having glycosyltransfase activity on terpenoids or terpenoid glycoside scaffolds are described herein, including the UGT enzymes defined by SEQ ID NOS: 13 to 39, 46, 54, 60, 71 to 84, and 99. See Tables 1, 8, and 9.

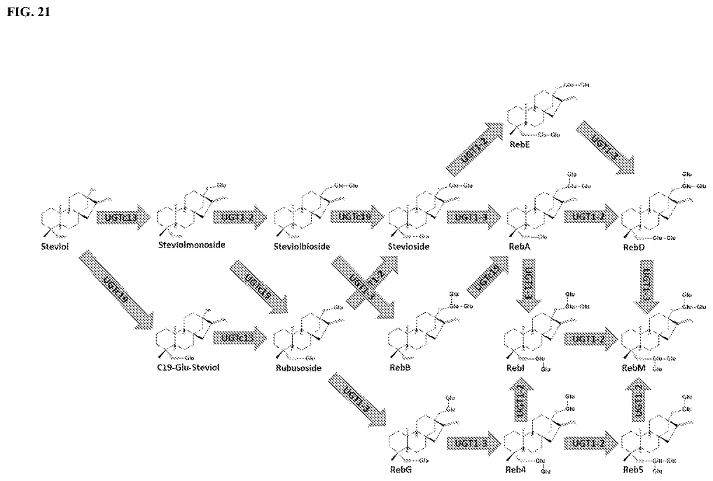

For example, in some embodiments, the glycosylated product is a rebaudioside (steviol glycoside). In these embodiments, the UGT enzymes are capable of one or more of primary glycosylation at the C13 and/or C19 hydroxyl of a steviol core; 1-2 branching glycosylations of the C13 and/or C19 primary glycosyl groups; and 1-3 branching glycosylations of the C13 and/or C19 primary glycosyl groups. See FIG. 21. In some embodiments, the UGT enzymes are selected from enzymes comprising amino acid sequences having at least about 70% sequence identity (or at least about 80%, at least about 85%, at least about 90%, at least about 95%, or at least about 97% sequence identity) to one of SEQ ID NOS: 13 to 32, and 84.

UGT enzymes for glycosylation of steviol and steviol glycosides (including for biosynthesis of RebM) are disclosed in US 2017/0332673 and 2020/0087692, which are hereby incorporated by reference in their entireties. Exemplary UGT enzymes are listed in Table 1, below:

TABLE 1
Example UGT Enzymes for Steviol Glycoside Production

| Type of | | |
|---|---|---|
| glycosylation | Enzyme Name | SEQ ID NO |
| C13 | SrUGT85C2 | 17 |
| | MbUGTC13 | 32 |
| C19 | SrUGT74G1 | 18 |
| | MbUGTc19 | 30 |
| | MbUGTc19-2 | 31 |
| | At75D1 | 99 |
| 1-2′ | SrUGT91D1 | 26 |
| | SrUGT91D2 | 27 |
| | SrUGT91D2e | 28 |
| | OsUGT1-2 | 29 |
| | MbUGT1, 2.2 | 13 |
| | MbUGT1, 2.3 | 14 |
| | MbUGT1, 2.4 | 15 |
| | MbUGT1, 2.5 | 16 |
| 1-3′ | SrUGT76G1 | 19 |
| | MbUGT1-3 | 20 |
| | 76G1_L200A | 21 |
| | MbUGT1-3_0 | 22 |
| | MbUGT1-3_1 | 23 |
| | MbUGT1-3_2 | 24 |
| | MbUGT1-3_3 | 25 |

In some embodiments, the glycosylated product is a mogroside. In various embodiments, the UGT enzymes are capable of one or more of primary glycosylation at the C3 and/or C24 hydroxyl of a mogrol core, 1-2 branching glycosylations of the C3 and/or C24 primary glycosyl groups; and/or 1-6 branching glycosylations of the C3 and/or C24 primary glycosyl groups. UGT enzymes useful for these embodiments are shown in Tables 8 and 9. In some embodiments, the UGT enzymes are selected from enzymes comprising amino acid sequences having at least about 70% sequence identity (or at least about 80%, at least about 85%, at least about 90%, at least about 95%, or at least about 97% sequence identity) to one of SEQ ID NOS: 13 to 17, 29, 33 to 39, 46, 54, 60, 71 to 80, and 82 to 84.

Changes to the amino acid sequence of an enzyme can alter its activity or have no measurable effect. Silent changes with no measurable effect are often conservative substitutions and small insertions or deletions on solvent-exposed surfaces that are located away from active sites and substrate-binding sites. In contrast, enzymatic activity is more likely to be affected by non-conservative substitutions, large insertions or deletions, and changes within active sites, substrate-binding sites, and at buried positions important for protein folding or conformation. Changes that alter enzymatic activity may increase or decrease the reaction rate or increase or decrease the affinity or specificity for a particular substrate. For example, changes that increase the size of a substrate-binding site may permit an enzyme to act on larger substrates and changes that position a catalytic amino acid side chain closer to a target site on a substrate may increase the enzymatic rate.

In some embodiments “rational design” is involved in constructing specific mutations in enzymes. Rational design refers to incorporating knowledge of the enzyme, or related enzymes, such as its reaction thermodynamics and kinetics, its three-dimensional structure, its active site(s), its substrate(s) and/or the interaction between the enzyme and substrate, into the design of the specific mutation. Based on a rational design approach, mutations can be created in an enzyme which can then be screened for increased production of a terpene or terpenoid relative to control levels. In some embodiments, mutations can be rationally designed based on homology modeling. As used herein, “homology modeling” refers to the process of constructing an atomic resolution model of one protein from its amino acid sequence and a three-dimensional structure of a related homologous protein.

Identity of amino acid sequences, i.e. the percentage of sequence identity, can be determined via sequence alignments. Such alignments can be carried out with several known algorithms, such as that described by Karlin and Altschul (Karlin & Altschul (1993) Proc. Natl. Acad. Sci. USA 90: 5873-5877), with hmmalign (HMMER package) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST protein alignments may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used.

The UGT enzymes or other expressed enzymes may be integrated into the chromosome of the microbial cell, or alternatively, are expressed extrachromosomally. For example, the UGT enzymes may be expressed from a bacterial artificial chromosome (BAC) or yeast artificial chromosome (YAC).

The amino acid sequence of one or more of the UGT enzymes (or other expressed enzymes) can optionally include an alanine inserted or substituted at position 2 to decrease turnover in the cell. In various embodiments, one or more UGT enzymes comprise an alanine amino acid residue inserted or substituted at position 2 to provide additional stability in vivo.

Expression of enzymes can be tuned for optimal activity, using, for example, gene modules (e.g., operons) or independent expression of the enzymes. For example, expression of the genes or operons can be regulated through selection of promoters, such as inducible or constitutive promoters, with different strengths (e.g., strong, intermediate, or weak). Several non-limiting examples of promoters of different strengths include Trc, T5 and T7. Additionally, expression of genes or operons can be regulated through manipulation of the copy number of the gene or operon in the cell. In some embodiments, the cell expresses a single copy of each UGT enzyme. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome.

Optimization of expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In some embodiments, the glycosylated product comprises RebM. In these embodiments, the UGT enzymes are capable of primary glycosylation at the C13 and C19 hydroxyl of a steviol core; 1-2 branching glycosylations of the C13 and C19 primary glycosyl groups; and 1-3 branching glycosylations of the C13 and C19 primary glycosyl groups. See FIG. 21. In some embodiments, the UGT enzymes are selected from enzymes comprising amino acid sequences having at least about 70% sequence identity (or at least about 80%, at least about 85%, at least about 90%, at least about 95%, or at least about 97% sequence identity) to one of SEQ ID NOS: 13 to 32, and 84. In such embodiments, the glycosylated product recovered according to this disclosure is at least about 50% RebM, or at least about 75% RebM, or at least about 85% RebM, or at least about 90% RebM, or at least about 95% RebM, with respect to the total steviol glycoside component.

In some embodiments, the glycosylated product comprises RebE and/or RebD. In such embodiments, the bacterial cell may express one or more UGT enzymes capable of 1-2 glycosylation of steviol C13 and C19 primary glycosyl groups. In some embodiments, the substrates for glycosylation comprise RebA and stevioside as major components (e.g., at least about 20%, or at least about 30%, or at least about 50%, or at least about 70% of the steviol glycoside composition of the substrate). In some embodiments, the UGT enzymes are selected from enzymes having at least about 70% sequence identity (or at least about 80%, at least about 85%, at least about 90%, at least about 95%, or at least about 97% sequence identity) to one of SEQ ID NOS: 13 to 16 and 26 to 29. In such embodiments, the glycosylated product recovered according to this disclosure is at least about 50% RebE and/or RebD, or at least about 75% RebE and/or RebD, or at least about 85% RebE and/or RebD, or at least about 90% RebE and/or RebD, or at least about 95% RebE and/or RebD, with respect to the total steviol glycoside component.

In some embodiments, the glycosylated product comprises RebB. In such embodiments, the bacterial cell expresses one or more UGT enzymes capable of deglycosylation of steviol C19 primary glycosyl groups. In some embodiments, the substrates for glycosylation comprise RebA as major a component (e.g., at least about 20%, or at least about 30%, or at least about 50%, or at least about 70% of the rebaudioside composition of the substrate). In some embodiments, the UGT enzymes are selected from enzymes having at least about 70% sequence identity (or at least about 80%, at least about 85%, at least about 90%, at least about 95%, or at least about 97% sequence identity) to one of SEQ ID NOS: 18, 30, 31 and 99. In some embodiments, the bacterial cell expresses a UGT enzyme having at least about 70% (or at least about 80%, at least about 85%, at least about 90%, at least about 95%, or at least about 97% sequence identity) sequence identity to SEQ ID NO: 31 or SEQ ID NO: 99. In such embodiments, the glycosylated product recovered according to this disclosure is at least about 50% RebB, or at least about 75% RebB, or at least about 85% RebB, or at least about 90% RebB, or at least about 95% RebB, with respect to the total steviol glycoside component.

In some embodiments, the glycosylated product comprises RebI. In such embodiments, the bacterial cell expresses one or more UGT enzymes capable of 1-3 glycosylation of a steviol C19 primary glycosyl groups. In some embodiments, the substrates for glycosylation comprise RebA as major a component (e.g., at least about 20%, or at least about 30%, or at least about 50%, or at least about 70%, or at least about 80% of the steviol glycoside composition of the substrate). In some embodiments, the UGT enzymes are selected from enzymes having at least about 70% (or at least about 80%, at least about 85%, at least about 90%, at least about 95%, or at least about 97%) sequence identity to one of SEQ ID NOS: 19 to 25. In such embodiments, the glycosylated product recovered according to this disclosure is at least about 50% RebI, or at least about 75% RebI, or at least about 85% RebI, or at least about 90% RebI, or at least about 95% RebI, with respect to the total steviol glycoside component.

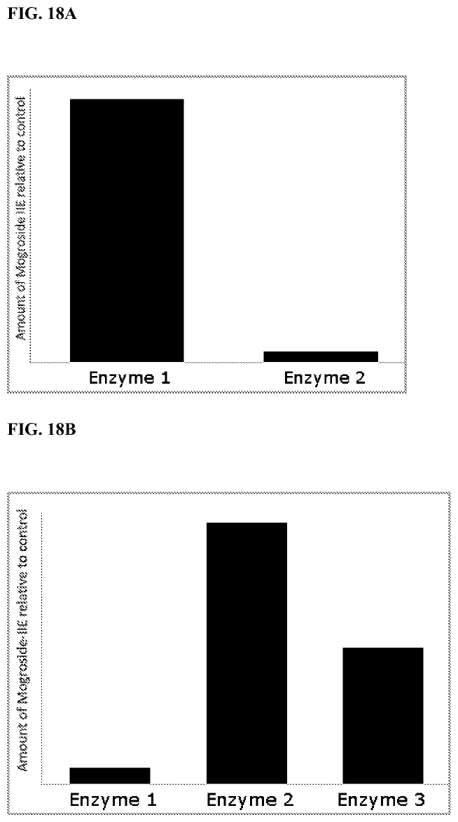

In some embodiments, the substrate is provided as a monk fruit extract or fraction thereof, or a biosynthetically produced mogrol or mogrol glycoside. For example, the substrate may comprise one or more substrates selected from mogrol, mog. I-A, mog. I-E, mog. II-A, mog. II-E, mog III, mog IVA, mog. IV, and siamenoside. The glycosylated product may comprise, for example, one or more of mog. IV, mog. IVA, mog. V, mog. VI, isomog. V, and siamenoside. See FIG. 18. In various embodiments, the UGT enzymes are capable of one or more of primary glycosylation at the C3 and/or C24 hydroxyl of a mogrol core, and 1-2 branching glycosylations of the C3 and/or C24 primary glycosyl groups; and 1-6 branching glycosylations of the C3 and/or C24 primary glycosyl groups. In some embodiments, the glycosylated product is mog. V or siamenoside. In various embodiments, the UGT enzymes are selected from enzymes comprising amino acid sequences having at least about 70% (or at least about 80%, at least about 85%, at least about 90%, at least about 95%, or at least about 97%) sequence identity to one of SEQ ID NOS: 13 to 17, 29, 33 to 39, 46, 54, 60, 71 to 80, and 82 to 84. See Tables 8 and 9. In various embodiments, the method results in at least about 40% conversion of the substrate to the glycosylated product, or at least about 50% conversion of the substrate to the glycosylated product, or at least about 75% conversion of the substrate to the glycosylated product, or at least about 90% conversion of the substrate to the glycosylated product, or at least about 95% conversion of the substrate to the glycosylated product (with respect to the total mogrol glycoside component of the recovered composition).

In various embodiments, the bacterial cell biomass is created by growth in complex or minimal medium. The bacterial cell is then cultured in the presence of the substrate for glycosylation with one or more carbon sources. In some embodiments, the carbon source comprises one or more of glucose, sucrose, fructose, xylose, and glycerol. In some embodiments, the carbon sources include sucrose, and one or more of glycerol and glucose. In general, suitable carbon sources include C1 to C6 carbon sources. Culture conditions can be selected from aerobic, microaerobic, and anaerobic. The culturing may be performed in batch, continuous, or semi-continuous processes. For example, in some embodiments, the method is conducted as a fed batch process.

In some embodiments, the substrates are incubated with the bacterial cell for about 72 hours or less, or for about 48 hours or less. In certain embodiments, the substrates are incubated with the bacterial cell from 1 to about 3 days, using, for example, a stirred tank fermenter. In various embodiments, the glycoside products are recovered as described elsewhere herein. For example, recovery may comprise one or more of: lowering the pH of the culture to below about pH 5 or raising the pH to above about pH 9, raising the temperature to at least about 50° C., and addition of one or more glycoside solubility enhancers; followed by enzyme or biomass removal.

In other aspects and embodiments, the invention provides an engineered UDP-dependent glycosyltransferase (UGT) enzyme with high productivity for glycosylating substrates, including terpenoid glycoside substrates, and including in connection with the bacterial cells and methods described herein. In some embodiments, the engineered UGT enzyme comprises an amino acid sequence that has at least about 70% sequence identity (or at least about 80%, at least about 85%, at least about 90%, at least about 95%, or at least about 97% sequence identity) to SEQ ID NO: 13, and having one or more amino acid modifications that improve glycosylating activity on terpenoid glycoside substrates (e.g., steviol glycoside substrates). In some embodiments the amino acid modifications comprise one or more (e.g., at least 2, 3, 4, 5, 6, 7, 8, 9, or 10) amino acid substitutions selected from: V397S, V397C, G5N, S20E, S23D, R45Y, H59P, G94S, K97E, M150L, I185F, A206P, G210E, Q237R, M250K, A251E, C252L, G259E, Q263Y, I287M, C288F, V336L, F338L, D351E, F186I, F186M, F186T, L418F, A451T, A451L, T453K, T453R, V456S, V456W, V456T, V456M with respect to SEQ ID NO: 13. See Table 2. Alternatively or in addition, the amino acid modifications comprise the substitution of residues 270 to 281 of SEQ ID NO: 13 with from five to fifteen amino acids comprised predominately of glycine and serine amino acids. Alternatively or in addition, the amino acid modifications comprise insertion of one or two amino acids at position 3 with respect to SEQ ID NO: 13, and/or addition of an amino acid to the C-terminus of SEQ ID NO: 13.

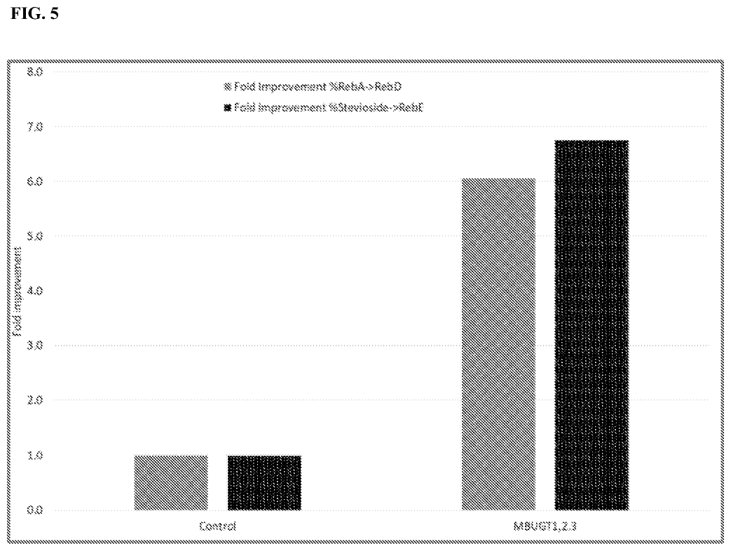

In some embodiments, the UGT enzyme has a substitution of amino acids 270 to 281 of SEQ ID NO: 13 with the sequence GGSGGS (SEQ ID NO: 85). In these or other embodiments, the UGT enzyme has an insertion of Arg at position 3, or an insertion of Ile-Arg between positions 2 and 3 with respect to SEQ ID NO: 13. In these or other embodiments, the UGT enzyme comprises one or more (or all) substitutions selected from G5N, F186T, and V397S with respect to SEQ ID NO: 13. An exemplary UGT enzyme of this aspect comprises the amino acid sequence of SEQ ID NO: 14. See FIG. 5.

In still other embodiments, the UGT enzyme comprises an amino acid sequence that has at least about 70% (or at least about 80%, at least about 85%, at least about 90%, at least about 95%, or at least about 97%) sequence identity to SEQ ID NO: 14, and having one or more amino acid modifications that improve glycosylating activity on terpenoid glycoside substrates (e.g., steviol glycoside substrates). For example, the amino acid modifications may comprise one or more (e.g., at least 2, 3, 4, 5, 6, 7, 8, 9, or 10) amino acid substitutions selected from: V395A, Q263Y, D269R, K97E, Q262E, H59P, G259E, M150L, Y267H, T3R, V95Q, A238E, S308Q, Q237R, R45Y, E254D, L203L, S151R, S123D, D351E, T453M, G94T, T186M, V336L, L58S, F338L, F51W, C252L, M250D, A251E, C252V, A79P, W401F, S323A, A251E, A130D, S42E, H400Y, S266R, S23D, P56A, A206P, M250K, A143W, V456T, G94S, I427F, T186L, T453F, C252R, V38F, R45F, T37S, Q244K, L11I, I287M, V31P, T43D, and P39T, with respect to SEQ ID NO: 14. See Table 3. Alternatively or in addition, the amino acid modifications comprise a deletion of residues 270 to 281 of SEQ ID NO: 14, with a linker of from five to fifteen amino acids and comprised predominately of glycine and serine amino acids. Alternatively or in addition, the UGT enzyme comprises an insertion of one or two amino acids at position 3 with respect to SEQ ID NO: 14, and/or addition of an amino acid to the C-terminus of SEQ ID NO: 14.

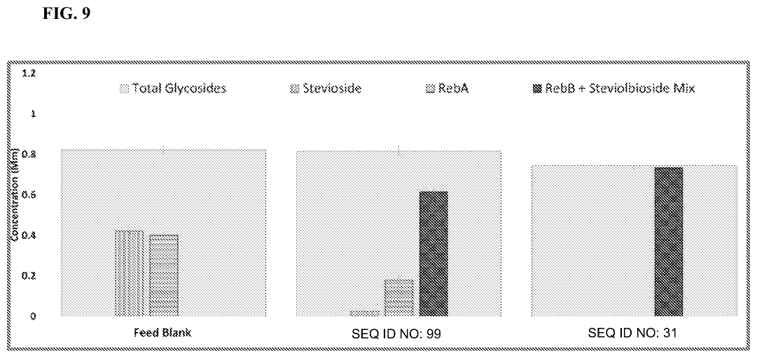

In some embodiments, the UGT enzyme has a substitution of amino acids 270 to 281 of SEQ ID NO: 14 with a linker sequence of from 6 to 12 amino acids composed predominately of Ser and Gly. In these or other embodiments, the UGT enzyme comprises one or more substitutions (or all substitutions) selected from H59P, A238E, and L417F with respect to SEQ ID NO: 14. In these or other embodiments, the UGT enzyme comprises an insertion or Arg-Arg between A2 and T3 of SEQ ID NO: 14. An exemplary UGT enzyme according to these embodiments comprises the amino acid sequence of SEQ ID NO: 15. See FIG. 9.

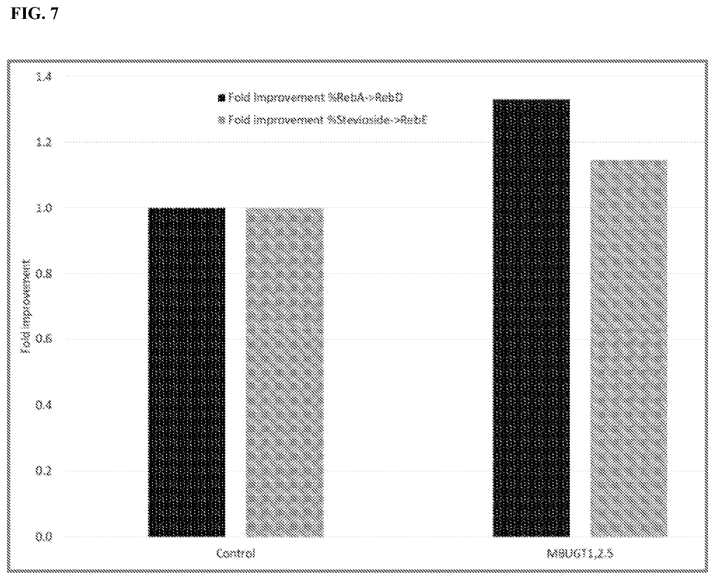

In still other embodiments, the UGT enzyme comprises an amino acid sequence that has at least about 70% (or at least about 80%, at least about 85%, at least about 90%, at least about 95%, or at least about 97%) sequence identity to SEQ ID NO: 15, and having one or more amino acid substitutions that improve glycosylating activity on terpenoid glycoside substrates (e.g., steviol glycoside substrates). Such amino acid substitutions may be at positions selected from 125, 152, 153, and 442 with respect to SEQ ID NO: 15. In some embodiments, the UGT enzyme comprises one or more (or all) amino acid substitutions selected from M152A, S153A, P442D, and S125V with respect to SEQ ID NO: 15. See Table 4. In exemplary UGT enzyme according to these embodiments comprises the amino acid sequence of SEQ ID NO: 16. See FIG. 7.

In other aspects and embodiments, the invention provides UGT enzymes (including bacterial cells expressing the same) for glycosylating a mogrol or mogrol glycoside substrate. In these embodiments, the method comprises contacting the substrate with a UGT enzyme in the presence of UDP-sugar (e.g., UDP-glucose). The UGT enzyme may comprise an amino acid sequence that has at least about 80% (or at least about 85%, at least about 90%, at least about 95%, or at least about 97%, or at least about 98%, or at least about 99%) sequence identity to an amino acid sequence selected from: SEQ ID NO: 84, SEQ ID NO: 80, SEQ ID NO: 46, SEQ ID NO: 83, SEQ ID NO: 82, SEQ ID NO: 73, SEQ ID NO: 72, SEQ ID NO: 78, SEQ ID NO: 54, SEQ ID NO: 74, SEQ ID NO: 75, SEQ ID NO: 76, SEQ ID NO: 13, SEQ ID NO: 14, SEQ ID NO: 15, SEQ ID NO: 16, SEQ ID NO: 29, and SEQ ID NO: 79. See Tables 8 and 9.

In various embodiments, the substrate is contacted with a UGT enzyme comprising an amino acid sequence that has at least about 80% (or at least about 85%, at least about 90%, at least about 95%, or at least about 97%, or at least about 98%, or at least about 99%) sequence identity to an amino acid sequence selected from: SEQ ID NO: 84, SEQ ID NO: 80, SEQ ID NO: 83, SEQ ID NO: 73, SEQ ID NO: 72, SEQ ID NO: 54, and SEQ ID NO: 13.

In these embodiments, the mogrol or mogrol glycoside substrate may be provided as a plant extract or fraction thereof, such as a monkfruit extract or fraction thereof. For example, the substrate may comprise (or be enriched for) one or more substrates selected from mogrol, mog. I-A, mog. I-E, mog. II-A, mog. II-E, mog III, mog IVA, mog. IV, and siamenoside. In these embodiments, the glycosylated product may comprise one or more of mog. IV, mog. IVA, mog. V, mog. VI, isomog V, and siamenoside. For example, the UGT enzymes may be capable of one or more of primary glycosylation at the C3 and/or C24 hydroxyl of a mogrol core, and/or 1-2 and/or 1-6 branching glycosylations of the C3 and/or C24 primary glycosyl groups. An exemplary product according to these embodiments is mog. V. Other mogroside products can be prepared (including mog. IV, mog. VI, and siamenoside), and UGT enzymes selected by their glycosylation activity.

In some embodiments with regard to producing mogrol glycosides, the substrates are cultured with a microbial cell expressing the UGT enzymes. Exemplary microbial cells include bacterial cells, such as a species of Escherichia, Bacillus, Rhodobacter, Zymomonas, or Pseudomonas. Exemplary bacterial cells include Escherichia coli, Bacillus subtilis, Rhodobacter capsulatus, Rhodobacter sphaeroides, Zymomonas mobilis, or Pseudomonas putida. In some embodiments, the bacterial cell is E. coli. In various embodiments, the bacterial cell is engineered for whole cell bioconversion processes as described herein, for example, having one or more genetic modifications that increase availability of UDP-sugar and/or expressing a sucrose synthase, as described elsewhere herein.

In still other embodiments, the microbial cell is a yeast cell, which may be selected from species of Saccharomyces, Pichia, or Yarrowia, including Saccharomyces cerevisiae, Pichia pastoris, and Yarrowia lipolytica.

However, in still other embodiments, the substrates are incubated with a cell lysate comprising the UGT enzymes, or are incubated with purified recombinant UGT enzymes according to know techniques. UDP-sugar to support the glycosylation reaction can be added exogenously.

In various embodiments, the glycosylated product is recovered according to methods described below. Such methods can comprise one or more of: lowering the pH of the reaction or culture to below about pH 5 or raising the pH of the reaction or culture to above about pH 9, raising the temperature to at least about 50° C., and adding one or more glycoside solubility enhancers; followed by enzyme or biomass removal.

In some aspects, the invention provides a method for producing and recovering a glycoside product. In such embodiments, the method comprises converting a substrate for glycosylation to a target glycoside product by enzymatic transfer of one or more sugar moieties in a cell-free reaction or in a microbial culture, which may optionally employ a method, UGT enzyme, and/or bacterial cell described herein. The method further comprises recovering the glycoside products from the reaction or culture, where the recovering comprising one or more of: lowering the pH of the reaction or culture to below about pH 5, raising the pH of the reaction or culture to above about pH 9, raising the temperature to at least about 50° C., and adding one or more glycoside solubilizers; followed by enzyme or biomass removal.

Conventionally, biomass removal is the first step in recovery, to remove large cellular debris, and to avoid further disruption of cells that would complicate downstream purification. However, in accordance with some embodiments of the present invention, the culture material will be highly viscous and difficult to process. For example, efficiency of biomass removal by centrifugation can be limited by the physical properties of the harvested culture material. By treating the culture material as described herein, prior to biomass or enzyme removal, it is possible to produce a product with desirable qualities, including: high purity of glycoside product, white color, easy solubilization, odorless, and high recovery yield. In particular, initial pH and temperature adjustment of the culture can change fluid characteristics of the broth, and increase efficiency of a disc stack separator for biomass removal. Further, solubility and therefore yield of glycoside product is substantially increased by the pH and temperature adjustment, which avoids significant losses of glycoside product in the solid phase.

In various embodiments, the glycosylated product is a terpenoid glycoside, such as one or more of RebM, RebE, RebD, RebB, and RebI (e.g., as discussed herein). In some embodiments, the glycosylated product is RebM. In other embodiments, the glycosylated product includes one or more of mog. IV, mog. IVA, mog. V, mog. VI, isomog. V, and siamenoside (as described herein). An exemplary mogroside product is mog. V.

In some embodiments, the enzymatic transfer occurs in a microbial culture, where the microbial culture comprises microbial strains expressing one or more UGT enzymes (e.g., whole cell bioconversion using fed substrate). In other embodiments, the microbial strain further expresses a biosynthetic pathway producing the substrate for glycosylation (e.g., steviol or mogrol), and expresses the one or more UGT enzymes for glycosylating the substrate. See, for example, U.S. Pat. No. 10,463,062 and WO 2019/169027, which are hereby incorporated by reference in their entireties. In various embodiments, the enzymatic transfer is by microbial culture of a yeast strain, such as those selected from Saccharomyces, Pichia, or Yarrowia, including Saccharomyces cerevisiae, Pichia pastoris, and Yarrowia lipolytica. In other embodiments, the enzymatic transfer is by microbial culture of a bacterial cell as described herein, including E. coli cells engineered for whole cell bioconversion in some embodiments (e.g., expressing one or more sucrose synthase enzymes, and/or comprising one or more genetic modifications that improve UDP-sugar availability, as described). For example, the bacterial cell may comprise genetic modifications, for example, where: ushA and galETKM or orthologs thereof are deleted, inactivated, or reduced in expression or activity; pgi or ortholog thereof is deleted, inactivated, or reduced in expression or activity; E. coli pgm (SEQ ID NO: 92) and/or ycjU (SEQ ID NO: 94) or ortholog or derivative thereof (e.g., a derivative having higher activity than the wild-type enzyme) are overexpressed; and/or E. coli galU (SEQ ID NO: 93) and/or Bifidobacterium bifidum ugpA (SEQ ID NO: 95) or orthologs or derivatives thereof (e.g., a derivative having higher activity than the wild-type enzyme) are overexpressed.

In some embodiments, the enzymatic transfer takes place in a bioreactor having a volume of at least about 10,000 L, or at least about 50,000 L, or at least about 100,000 L, or at least about 150,000 L, or at least about 200,000 L, or at least about 500,000 L. In various embodiments, culture material may be harvested for glycoside recovery in batch, continuous, or semi-continuous manner.

In some embodiments, the harvested culture material is pH adjusted, for example, to a pH within the range of about 2 to about 5. In some embodiments, the pH is adjusted to a pH in the range of about 2 to about 4, or a pH of about 2 to about 3.5, or a pH within the range of about 2.5 to about 4. In some embodiments, the pH is adjusted to about 2.5, about 3.0, or about 3.5. In still other embodiments, the pH is adjusted to the basic pH range, such as a pH within the range of about 9 to about 12, or within the range of about 9.5 to about 12 or about 10 to about 12 (e.g., about 11, about 11.5, or about 12). In various embodiments, pH adjustment improves glycoside solubility and/or improves the physical properties of the harvested material, so that biomass and/or enzymes are more easily removed without large loss in product. pH adjustment may be by addition or titration of an organic or inorganic acid or hydroxide ions, according to known methods.

Alternatively or in addition, the temperature of the harvested culture material is adjusted to a temperature between about 50° C. and about 90° C., such as from about 50° C. to about 80° C. For example, in some embodiments, the temperature is adjusted to a temperature in the range of about 55° C. to about 75° C., or a temperature in the range of about 65° C. to about 75° C. In some embodiments, the temperature is adjusted to about 70° C. In various embodiments, temperature adjustment improves glycoside solubility and/or improves the physical properties of the harvested material, so that biomass and/or enzymes are more easily removed without large loss in product. In some embodiments, temperature adjustment takes place by transfer of the reaction media or culture to pre-heated harvest tanks. In some embodiments, temperature adjustment takes place in-line, for example, by passage through a retention loop on the way to the next unit operation.

In some embodiments, harvested reaction or culture media is transferred from a reaction tank to a harvest tank for pH and/or temperature adjustment, which may take place in the same harvest tank or in different harvest tanks. In some embodiments, pH and/or temperature adjustment take place in-line, as a continuous unit operation. Temperature and pH adjustment can take place in any order or simultaneously. In some embodiments, pH adjustment takes place prior to temperature adjustment. In other embodiments, temperature adjustment takes place prior to pH adjustment. In still other embodiments, pH adjustment and temperature adjustment take place substantially simultaneously.

Alternatively or in addition, the method comprises adding one or more glycoside solubility enhancers. Exemplary solubility enhancers include chemical reagents with alcohol functional groups (including organic acids and polymers) and/or polar reagents (including those with ether, ester, aldehyde, and ketone functional groups), and including but not limited to glycerol, 1,3-propanediol, polyvinyl alcohol, polyethylene glycol, among others. Other exemplary solubility enhancers include organic acids, saccharides, and polysaccharides. Other solubility enhances are described in US 2020/0268026, which is hereby incorporated by reference in its entirety. Improvement in glycoside solubility facilitates biomass and/or enzyme removal without large loss in product. Generally, solubility enhancers can be added to the harvested culture material in a range of from about 0.1 wt % to about 2 wt %, such as in a range of from about 0.1 wt % to about 1 wt % (e.g., about 0.5 wt %).

Subsequently, biomass and/or enzymes are removed by centrifugation, thereby preparing a clarified broth. An exemplary process for biomass removal employs a disc stack centrifuge to separate liquid and solid phases. The clarified broth (liquid phase) is recovered for further processing to purify the glycoside product. The separated biomass (solid phase) can be reprocessed for further glycoside product recovery, or is alternatively processed as waste.

In some embodiments, glycosides are crystallized from the clarified broth. In some embodiments, the process includes 1, 2, or 3 crystallization steps. In some embodiments, glycoside products are purified from the clarified broth using one or more processes selected from filtration, ion exchange, activated charcoal, bentonite, affinity chromatography, and digestion, which can optionally be conducted prior to crystallization and/or prior to recrystallization. These processes can be selected to achieve a high product purity, attractive color (which is white in the case of RebM), easy solubilization, odorless, and high recovery yield. In some embodiments, the method employs affinity chromatography, such as with one or more of a styrene-divinylbenzene adsorbent resin, a strongly acidic cation exchange resin, a weakly acidic cation exchange resin, a strongly basic anion exchange resin, a weakly basic anion exchange resin, and a hydrophobic interaction resin. In still other embodiments, the process employs simulated moving bed chromatography, as described in U.S. Pat. No. 10,213,707, which is hereby incorporated by reference in its entirety. In still other embodiments, the recovery process is non-chromatographic (i.e., there are no chromatographic steps), providing substantial cost advantages. For example, the recovery process after biomass removal can consist essentially of, or consist of, filtration and crystallization steps. In some embodiments, the recovery process employs organic solvents (e.g., ethanol), but in other embodiments the process is entirely with aqueous solvents. In some embodiments, two crystallization steps are employed.

In some embodiments, the recovery process will include one or more steps of tangential flow filtration (TFF). For example, TFF with a filter having a pore size of about 5 kD can remove endotoxin, large proteins, and other cell debris, while also enhancing solubility of the final powdered product. In some embodiments, prior to initial crystallization, glycoside products are purified by tangential flow filtration, optionally having a membrane pore size of about 5 kD. TFF with a filter having a pore size of about 0.5 kD can also be employed downstream to remove small molecule impurities and salts, and/or to concentrate the mother liquor for recrystallization. In some embodiments, TFF with a pore size of about 0.5 kD is employed prior to recrystallization.

In each case, crystallization steps can include one or more phases of static crystallization, stirred crystallization, and evaporative crystallization. For example, crystallization steps may comprise a static phase followed by a stirred phase, to control crystal morphology. The static phase can grow large crystals with a high degree of crystalline domains. The crystallization process can include seeding crystals, or in some embodiments, does not involve seeding crystals (i.e., crystals form spontaneously). In various embodiments, the crystallization solvent comprises water or water/ethanol. Exemplary crystallization solvents include water, optionally with from about 5% to about 50% ethanol by volume, or from about 25% to about 50% ethanol by volume (e.g., from about 30% to about 40% ethanol by volume). In some embodiments, after seeding crystals during a static phase, a stirred phase will rapidly grow the crystals, and increase the degree of amorphous domains. Using this process, resulting crystals may have better final solubility and a high purity of glycoside product, and may be easier to recover and wash.

In various embodiments, prior to recrystallization, glycoside products are resolubilized in a solvent (such as but not limited to water and/or ethanol), which may employ one or more of: lowering the pH of the solvent and glycoside product solution or suspension to below about pH 5 or raising the pH of the solution or suspension to above about pH 9, heating to at least about 50° C., and adding one or more glycoside solubilizers. The targeted values for pH, temperature, glycoside solubilizer concentration can alternatively be as employed for biomass removal. For example, the glycoside product solution or suspension may be pH adjusted within the range of about 2 to about 5. In other embodiments, the pH is adjusted to the basic pH range, such as a pH within the range of about 9 to about 12, or within the range of about 9.5 or about 10 to about 12. pH adjustment may be by addition or titration of an organic or inorganic acid or hydroxide ions, according to known methods. In still other embodiments, recrystallization is performed at a pH of about 4 to about 12. Alternatively or in addition, the temperature of the solution or suspension is adjusted to a temperature between about 50° C. and about 90° C., such as from about 50° C. to about 80° C. Exemplary recrystallization solvents include water, optionally with from about 5% to about 50% ethanol by volume, or from about 25% to about 50% ethanol by volume (e.g., about 30% to about 40% ethanol by volume). Alternatively or in addition, solubility enhancers can be added to the solution/suspension in a range of from about 0.1 wt % to about 2 wt %, such as in a range of from about 0.1 wt/to about 1 wt % (e.g., about 0.5 wt %), as described. Exemplary solubility enhancers include glycerol.

In some embodiments, after crystallization, resulting crystals are isolated, e.g., using basket centrifuges or belt filter, thereby isolating glycoside wet cake (e.g., steviol glycoside or mogrol glycoside wet cake). Washing at basket centrifuge steps can employ washes with water, or alternatively other rinses can be employed (e.g., chilled water/ethanol). In some embodiments, the cake is dissolved and recrystallized. The wet cake from recrystallization may then be dried, optionally using a belt dryer, paddle dryer, or spray dryer. The dried cake can be milled and packaged.

Prior to recrystallization, the glycoside solution (e.g., RebM and other steviol glycosides) can be filtered to remove impurities. The filter can be about a 0.2 micron filter in some embodiments. Alternatively, other pore sizes can be employed, such as about 0.45 micron filters and about 1.2 micron filters. In accordance with embodiments, the material of the filter can be selected to further remove impurities, such as by adsorption. For example, hydrophilic materials such as polyethersulfone (PES) have significant advantages over more hydrophobic materials such as polypropylene. Other exemplary hydrophilic filer materials include nylon, cellulose acetate, cellulose nitrate, and normally hydrophobic materials that have been functionalized to result in a hydrophilic material (such PTFE or PVDF coated with fluoroalkyl terminated polyethylene glycol).

In various embodiments, the recovery process results in a highly pure composition of the target glycoside. For example, in some embodiments, the target glycoside product is at least about 75% of the recovered composition by weight. In some embodiments, the target glycoside product is at least about 80%, or at least about 90%, or at least about 95% of the recovered composition by weight. In exemplary embodiments, the yield of the glycosylated product is at least about 25 grams of product per liter of culture or reaction (g/L), or at least about 50 g/L, or at least about 75 g/L, or at least about 100 g/L, or at least about 125 g/L, or at least about 150 g/L, or at least about 200 g/L.

In some aspects, the invention provides methods for making a product comprising a glycosylated product, such as a steviol glycoside or mogrol glycoside (e.g., RebM or mog. V). The method comprises incorporating the glycoside product (produced according to this disclosure) into a product, such as a food, beverage, oral care product, sweetener, flavoring agent, or other product. Purified glycosides, prepared in accordance with the present invention, may be used in a variety of products including, but not limited to, foods, beverages, texturants (e.g., starches, fibers, gums, fats and fat mimetics, and emulsifiers), pharmaceutical compositions, tobacco products, nutraceutical compositions, oral hygiene compositions, and cosmetic compositions. Non-limiting examples of flavors for which the glycosides can be used in combination include lime, lemon, orange, fruit, banana, grape, pear, pineapple, mango, bitter almond, cola, cinnamon, sugar, cotton candy and vanilla flavors. Non-limiting examples of other food ingredients include flavors, acidulants, and amino acids, coloring agents, bulking agents, modified starches, gums, texturizers, preservatives, antioxidants, emulsifiers, stabilizers, thickeners and gelling agents.

In some aspects, the invention provides methods for making a sweetener product comprising a plurality of high-intensity sweeteners, said plurality including two or more of a steviol glycoside (e.g., RebM, RebE, RebD, RebI, or RebB), a mogroside (e.g., mog. IV, mog. IVA, mog. V, mog. VI, or isomog. V), sucralose, aspartame, neotame, advantame, acesulfame potassium, saccharin, sugar alcohol (e.g., erythritol or xylitol), tagatose, cyclamate, neohesperidin dihydrochalcone, gnetifolin E, and/or piceatannol 4′-O-β-D-glucopyranoside. The method may further comprise incorporating the sweetener product into a food, beverage, oral care product, sweetener, flavoring agent, or other product, including those described above.

Target glycoside(s), such as RebM or mog. V, and sweetener compositions comprising the same, can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory.

Further, target glycoside(s), such as RebM and mog. V, and sweetener compositions obtained according to this invention, may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, sweetener compositions can be used as a sweetener not only for drinks, foodstuffs, and other products dedicated for human consumption, but also in animal feed and fodder with improved characteristics.

Examples of products in which target glycoside(s) and sweetener compositions may be used include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others.

During the manufacturing of products such as foodstuffs, drinks, pharmaceuticals, cosmetics, tabletop products, and chewing gum, the conventional methods such as mixing, kneading, dissolution, pickling, permeation, percolation, sprinkling, atomizing, infusing and other methods may be used.

As used herein, the term “about” means f 10% of an associated numerical value.

Other aspects and embodiments of the invention will be apparent from the following Examples.

### EXAMPLES

### Example 1: Bioconversion Chassis Strain Engineering

Bioconversion (glycosylation) of steviol glycoside intermediates using engineered E. coli strains expressing UGT enzymes is described in US 2020/0087692, which is hereby incorporated by reference. US 2020/0087692 describes bacterial genetic modifications to increase the native flux to UDP-glucose, a critical substrate for the UGT enzymes. Greater than native flux to UDP-glucose allows for greater UGT performance by increasing the amount of substrate available to the UGTs. The genetic modifications result in the ability of the cell to convert fed substrates to glycosylated products, such as but not limited to rebaudiosides and mogrosides. Other substrates for glycosylation are described herein. Genetic modifications include: deletion or inactivation of enzymes that consume UDP-glucose (ushA, galETKM); deletion or inactivation of enzymes that consume a precursor of UDP-glucose, glucose-6-phosphate (G6P) (e.g., pgi); and overexpression of enzymes that convert G6P to UDP-glucose via glucose-1-phosphate (GIP) (e.g., pgm, galU). An E. coli strain having the modifications ΔushA, ΔgalETKM, Δpgi, and complementation of pgm and galU is referred to below as the “chassis strain.”

Additional chromosomal modifications were tested for improved bioconversion, as compared to the chassis strain. FIG. 1 shows improvement of steviol glycoside bioconversion from two additional chromosomal modifications. Fold improvement is with respect to total steviol glycoside conversion. Stevia leaf extract, sucrose, and glucose were fed to bioconversion strains expressing UGT enzymes of SEQ ID NOS: 15 and 25, and sucrose synthase of SEQ ID NO: 11. Bioconversion took place at 37° C. for 48 hr. Data were quantified by reverse phase LC DAD to quantify conversion of steviol glycosides. Deletion of otsA and otsB, shown as 1 in FIG. 1) showed an improvement in steviol glycoside bioconversion as compared to the chassis strain. Further, overexpression of ugpA (shown as 2 in FIG. 1) provided further improvement in total steviol glycoside bioconversion.

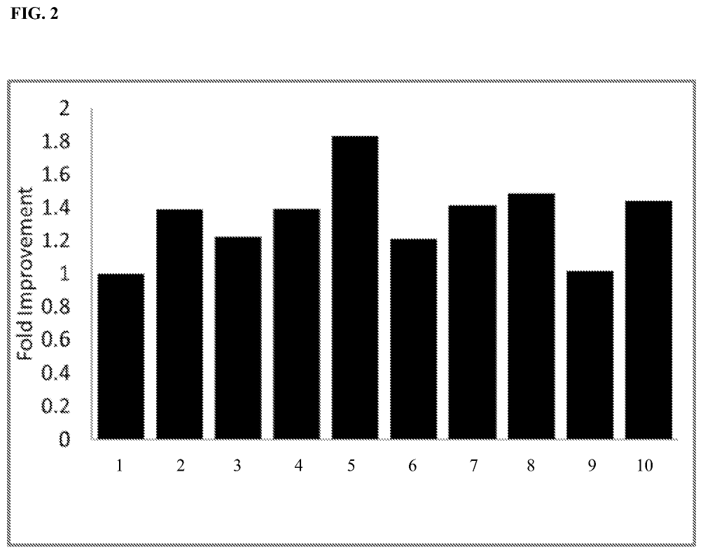

Other bacterial genes were overexpressed, and bioconversion tested as compared to the chassis strain expressing UGT enzymes of SEQ ID NOS: 14 and 24. FIG. 2 shows improvement of steviol glycoside bioconversion from a series of overexpressed genes complemented on plasmid. Fold improvement is with respect to total steviol glycoside conversion. Stevia leaf extract and glucose were fed to the bioconversion strains. Bioconversion took place at 37° C. for 48 hr. Data were quantified by reverse phase LC DAD to quantify conversion of steviol glycosides. As shown, several gene complementations improved overall glycosylation, including complementation with E. coli pgm (SEQ ID NO: 92), galU (SEQ ID NO: 93), pgm-galU (SEQ ID NOS: 92 and 93), ugpA (SEQ ID NO: 95), ycjU (SEQ ID NO: 94), adk (SEQ ID NO: 96), ndk (SEQ ID NO: 97), and cmk (SEQ ID NO: 98).

Strains were created that expressed candidate sucrose synthase enzymes, which may improve UDP-glucose availability when fed sucrose. Expression of a sucrose synthase enables splitting of sucrose into fructose and glucose. Glucose can be funneled towards UDP-glucose biosynthesis. However, the cells exhibit similar growth and UDP-glucose availability when grown on either glycerol or glucose as the carbon source. FIG. 3 shows improvement of steviol glycoside bioconversion from complemented sucrose synthases in the bioconversion chassis strain expressing UGT enzymes of SEQ ID NOS: 15 and 25. Genes were complemented on plasmid. Fold improvement is with respect to total steviol glycoside conversion. Stevia leaf extract, sucrose, and glucose were fed to the bioconversion strains. Bioconversion took place at 37° C. for 48 hr. Data were quantified by reverse phase LC DAD to quantify conversion of steviol glycosides. Several sucrose synthase enzymes provided dramatic improvements in glycosylation, including the enzymes represented by SEQ ID NOS: 2, 3, 5, 6, 7, 8, 9, 10, and 11.

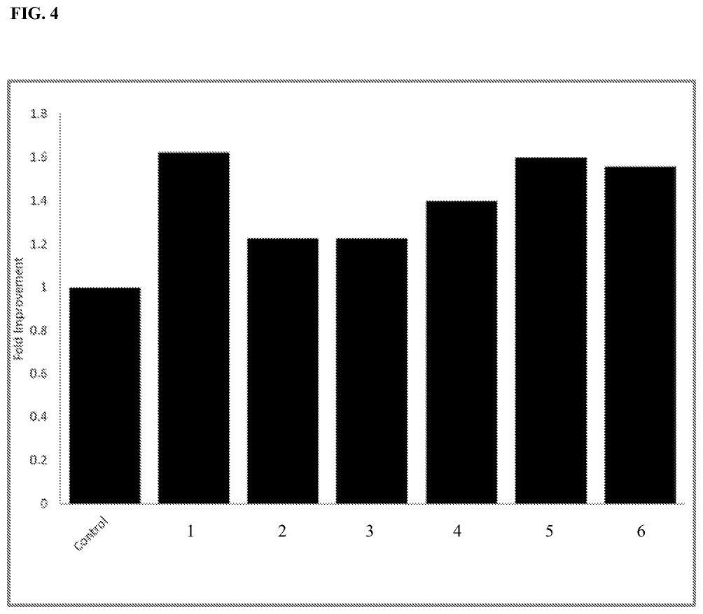

FIG. 4 shows improvement of steviol glycoside bioconversion by the bioconversion chassis strain expressing UGT enzymes of SEQ ID NO: 14 and SEQ ID NO: 24, with various gene knockouts. Fold improvement is with respect to total steviol glycoside conversion. Stevia leaf extract and glucose were fed to the bioconversion strains. Bioconversion took place at 37° C. for 48 hr. Data were quantified by reverse phase LC DAD to quantify conversion of steviol glycosides. Several gene knockouts were identified to improve bioconversion.

### Example 2: UGT Engineering and Target Steviol Glycoside Production

A UGT enzyme referred to as MbUGT1,2 is described in U.S. Pat. No. 10,743,567, which is hereby incorporated by reference. An engineered version of MbUGT1,2 (SEQ ID NO: 13) is described in US 2020/0087692, which is hereby incorporated by reference in its entirety. The UGT enzyme of SEQ ID NO: 13 was further engineered to improve activity for steviol glycoside bioconversion. FIG. 5 shows improvement of steviol glycoside bioconversion by the engineered version of SEQ ID NO: 14. Fold improvement is with respect to % steviol glycoside conversion of the parent UGT enzyme (SEQ ID NO: 13). SEQ ID NO: 14 has the following mutations from SEQ ID NO: 13: G5N, F186T, V397S. Stevia leaf extract and glucose were fed to the bioconversion strains. Bioconversion took place at 37° C. for 48 hr. Data were quantified by reverse phase LC DAD to quantify conversion of steviol glycosides.

Table 2 shows improvement of steviol glycoside bioconversion from individual mutations to the UGT enzyme of SEQ ID NO: 13. Fold improvement (FI) is with respect to % steviol glycoside conversion.

TABLE 2

| Mutation | FI % rebA −> rebD | FI % Stevioside −> rebE |
|---|---|---|
| G5N | 1.5 | 1.5 |
| S20E | 1.1 | 1.2 |
| S23D | 1.3 | 1.4 |
| R45Y | 1.5 | 1.6 |
| M55L | 1.0 | 1.1 |
| H59P | 1.8 | 1.8 |
| G94S | 1.2 | 1.3 |
| K97E | 1.4 | 1.4 |
| M150L | 1.5 | 1.4 |
| I185F | 1.3 | 1.2 |
| A206P | 1.3 | 1.2 |
| G210E | 1.2 | 1.2 |
| Q237R | 1.3 | 1.3 |
| A248G | 1.1 | 1.1 |
| M250K | 1.4 | 1.5 |
| A251E | 1.4 | 1.4 |
| C252L | 1.6 | 1.5 |
| G259E | 1.4 | 1.6 |
| Q263Y | 1.1 | 1.6 |
| I287M | 1.0 | 1.4 |
| C288F | 1.5 | 1.7 |
| l6* | 1.3 | 1.5 |
| V336I | 1.2 | 1.4 |
| F338L | 1.2 | 1.3 |
| D351E | 1.1 | 1.3 |
| V91A | 1.1 | 1.1 |
| F186I | 2.1 | 2.6 |
| F186M | 2.3 | 2.9 |
| F186T | 1.7 | 1.8 |
| V397S | 3.4 | 3.8 |
| V397C | 1.9 | 1.9 |
| L418F | 1.5 | 1.6 |
| A451T | 2.0 | 1.8 |
| A451L | 1.5 | 1.4 |
| T453K | 1.6 | 1.4 |
| T453R | 1.6 | 1.4 |
| V456S | 1.6 | 1.5 |
| V456W | 1.2 | 1.2 |
| V456T | 1.3 | 1.2 |
| V456M | 1.5 | 1.5 |
| P5A8 | 1.9 | 1.8 |
| P5A10 | 1.3 | 1.2 |
| P5B8 | 1.2 | 1.1 |
| P5B9 | 1.8 | 1.7 |
| P5E2 | 2.0 | 1.7 |

Key

| | | |
|---|---|---|
| Name | N-terminus | C-terminus |
| CTL | MATKG | IR |
| P5A8 | MARTKG | IR |
| P5A10 | MARTKG | IRTKG |
| P5B8 | MARTKG | I |
| P5B9 | MAIRTKG | — |
| P5E2 | MATKG | IRT |

*l6 has a GGSGGS linker (SEQ ID NO: 85). Linker swaps replace residues 270-281 of SEQ ID NO: 13.

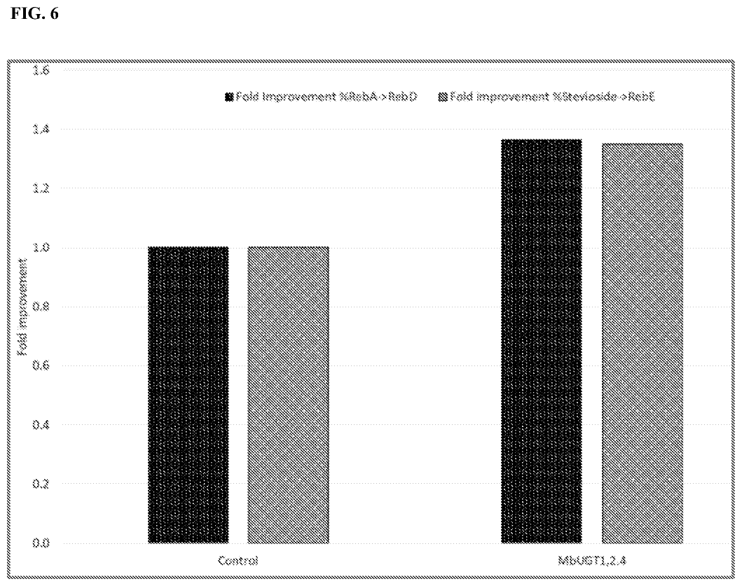

FIG. 6 shows improvement of steviol glycoside bioconversion by a further engineered version, SEQ ID NO: 15. Fold improvement is with respect to % steviol glycoside conversion of the parent UGT enzyme of SEQ ID NO: 14. The UGT enzyme of SEQ ID NO: 15 has the following mutations from SEQ ID NO: 14: ins_A2_T3_RR, H59P, A238E, L417F. Stevia leaf extract and glucose were fed to the bioconversion strains. Bioconversion took place at 37° C. for 48 hr. Data were quantified by reverse phase LC DAD to quantify conversion of steviol glycosides.

Table 3 shows improvement of steviol glycoside bioconversion from individual mutations to the UGT enzyme of SEQ ID NO: 14. Fold improvement (FI) is with respect to % steviol glycoside conversion.

TABLE 3

| | FI | FI |
|---|---|---|
| Mutation | %rebA→rebD | %Stevioside→rebE |
| V395A | 2.5 | 2.8 |
| ins_A2_T3_RR | 2.0 | 2.4 |
| Q263Y | 2.0 | 2.3 |
| 18* | 1.9 | 2.1 |
| D269R | 1.7 | 2.0 |
| 19* | 1.7 | 2.0 |
| K97E | 1.7 | 1.9 |
| Q262E | 1.7 | 2.0 |
| H59P | 1.7 | 2.0 |
| G259E | 1.7 | 1.9 |
| M150L | 1.7 | 1.9 |
| Y267H | 1.6 | 1.8 |
| T3R | 1.6 | 1.9 |
| V95Q | 1.6 | 1.8 |
| A238E | 1.6 | 1.9 |
| ins_A2_T3_R | 1.6 | 1.9 |
| S308Q | 1.6 | 1.6 |
| Q237R | 1.5 | 1.8 |
| R45Y | 1.5 | 1.9 |
| E254D | 1.5 | 1.7 |
| L203I | 1.5 | 1.6 |
| S151R | 1.5 | 1.6 |
| 17* | 1.5 | 1.7 |
| S123D | 1.5 | 1.7 |
| D351E | 1.5 | 1.6 |
| 112* | 1.5 | 1.7 |
| T453M | 1.5 | 1.7 |
| G94T | 1.4 | 1.6 |
| 110* | 1.4 | 1.7 |
| T186M | 1.4 | 1.5 |
| V336I | 1.4 | 1.5 |
| L58S | 1.4 | 1.5 |
| 111* | 1.4 | 1.5 |
| V336I_F338L | 1.4 | 1.5 |
| F51W | 1.4 | 1.7 |
| 16* | 1.4 | 1.6 |
| C252L | 1.4 | 1.6 |
| M250D_A251E_C252V | 1.4 | 1.5 |
| A79P | 1.3 | 1.4 |
| W401F | 1.3 | 1.3 |
| S323A | 1.3 | 1.3 |
| A251E | 1.3 | 1.5 |
| A130D | 1.3 | 1.4 |
| S42E | 1.3 | 1.8 |
| H400Y | 1.3 | 1.2 |
| S266R | 1.3 | 1.4 |
| S23D | 1.3 | 1.5 |
| P56A | 1.3 | 1.5 |
| A206P | 1.3 | 1.4 |
| M250K | 1.3 | 1.5 |
| F338L | 1.3 | 1.3 |
| A143W | 1.3 | 1.3 |
| V456T | 1.3 | 1.4 |
| G94S | 1.24 | 1.33 |
| I427F | 1.24 | 1.15 |
| T186I | 1.24 | 1.48 |
| M250K_A251E_C252L | 1.23 | 1.40 |
| T453F | 1.20 | 1.32 |
| C252R | 1.19 | 1.25 |
| V38F | 1.19 | 1.33 |
| R45F | 1.18 | 1.35 |
| T37S | 1.17 | 1.35 |
| Q244K | 1.16 | 1.25 |
| V26I | 1.16 | 1.18 |
| L11I | 1.16 | 1.39 |
| I152L | 1.15 | 1.09 |
| R145S | 1.14 | 1.06 |
| I287M | 1.14 | 1.30 |
| G94R | 1.13 | 1.12 |
| L418F | 1.09 | 0.93 |
| I185F | 1.08 | 0.73 |
| V31P | 1.08 | 1.22 |
| T43D | 1.08 | 1.29 |
| H99T | 1.04 | 0.76 |
| C299A | 1.03 | 0.76 |
| P39T | 0.98 | 1.31 |
| L417F | 1.24 | 1.34 |
| V395A | 1.24 | 1.25 |
| L418L (CTGαCTC) | 1.13 | 1.08 |
| S29T | 1.00 | 1.05 |
| L417M | 1.02 | 1.02 |
| C288C (TGTαTGC) | 1.03 | 1.00 |

Key

| Name | Linker |
|---|---|
| 16 | GGSGGS (SEQ ID NO: 85) |
| 17 | GGSGGSG (SEQ ID NO: 86) |
| 18 | GGSGGSGG (SEQ ID NO: 87) |
| 19 | GGSGGSGGS (SEQ ID NO: 88) |
| 110 | GGSGGSGGSG (SEQ ID NO: 89) |
| 111 | GGSGGSGGSGG (SEQ ID NO: 90) |
| 112 | GGSGGSGGSGGS (SEQ ID NO: 91) |

FIG. 7 shows improvement of steviol glycoside bioconversion by a further engineered version, SEQ ID NO: 16. Fold improvement is with respect to % steviol glycoside conversion of the parent UGT enzyme of SEQ ID NO: 15. SEQ ID NO: 16 has the following mutations from SEQ ID NO: 15: M152A, S153A. Stevia leaf extract and glucose were fed to the bioconversion strains. Bioconversion took place at 37° C. for 48 hr. Data were quantified by reverse phase LC DAD to quantify conversion of steviol glycosides. Table 4 shows improvement of steviol glycoside bioconversion from individual mutations to SEQ ID NO: 15. Fold improvement (FI) is with respect to % steviol glycoside conversion.

TABLE 4

| Mutation | FI % rebA −> rebD | FI % Stevioside −> rebE |
|---|---|---|
| M152A_S153A | 1.26 | 1.32 |
| P442D | 1.19 | 1.18 |
| S125V | 1.13 | 1.16 |
| G96M | 1.10 | 1.10 |
| H402W | 1.09 | 1.07 |
| K99F | 1.08 | 1.08 |
| S310T | 1.04 | 1.04 |
| A81K | 1.04 | 1.02 |
| L205Y | 1.04 | 1.03 |
| A81G | 1.04 | 1.09 |
| G189Y | 1.04 | 1.04 |
| K99E | 1.03 | 1.02 |
| M425S | 1.02 | 1.03 |
| A451R | 1.02 | 0.99 |
| A81K | 1.02 | 1.01 |
| P442G | 1.01 | 1.01 |
| G96M | 1.01 | 1.03 |
| L298F | 0.97 | 1.01 |

In addition to production of RebM with co-expression of four UGT enzymes (see FIG. 21), in various embodiments, other target steviol glycosides can be produced with expression of fewer UGTs and/or by utilizing certain rebaudiosides as substrates. For example, certain stevia leaf extracts can contain high amounts of RebA and stevioside. Expression of a 1-2 branching UGT enzyme (e.g., one of SEQ ID NOS: 13-16) will result in production of a RebE and RebD mix, depending on the rebaudioside content of the extract. Expression of a 1-3 branching UGT enzyme (e.g., SEQ ID NOS: 19-25) will produce substantially RebI. Further, expression of UGTC19 favors deglycosylation to steviolbioside and RebB.

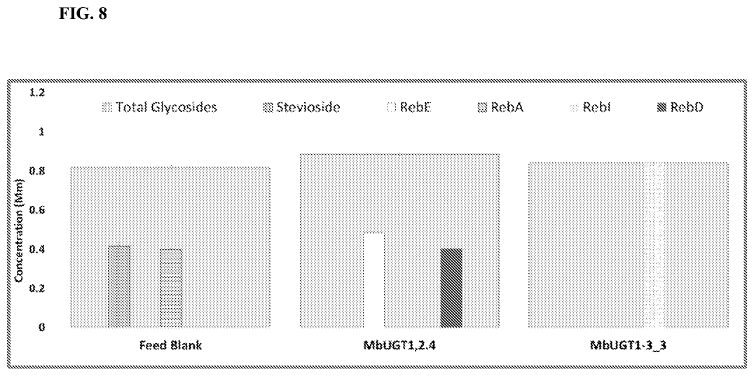

FIG. 8 demonstrates bioconversion of stevia leaf extract to a mix of RebE and RebD using the UGT enzyme of SEQ ID NO: 15, and conversion to RebI using the UGT enzyme of SEQ ID NO: 25. Stevia leaf extract (containing predominantly stevioside and RebA), sucrose, and glucose were fed to the bioconversion strains. Bioconversion took place at 37° C. for 48 hr. Data were quantified by reverse phase LC DAD to quantify conversion of steviol glycosides.

FIG. 9 shows bioconversion of stevia leaf extract to a mix of RebB and steviolbioside using the UGT enzyme of SEQ ID NO: 31 and SEQ ID NO: 99. Stevia leaf extract, UDP, and glucose were fed to the bioconversion strains. Bioconversion took place at 37° C. for 48 hr. Data were quantified by reverse phase LC DAD to quantify conversion of steviol glycosides.

### Example 3: Recovery of Steviol Glycosides

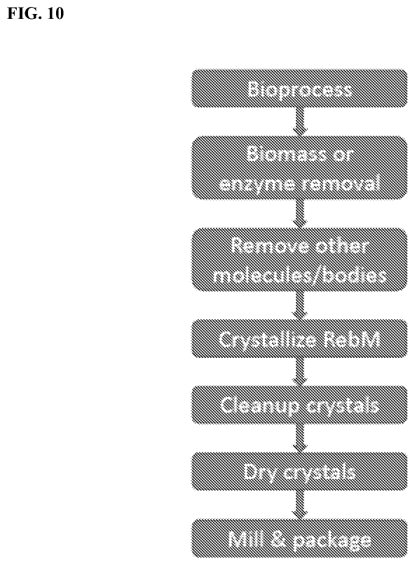

Conventionally, biomass (in the case of fermentation or whole cell/lysate bioconversion processes) or enzymes (in the case of bioconversion with purified enzymes) will be initially removed from the culture to allow for steviol glycoside (or other glycoside product) recovery and purification. Conventionally, it is preferred to remove biomass as an initial step in recovery to minimize disruption of microbial cells, in which cellular debris (both large and small molecules) would otherwise complicate the purification process. A conventional process for recovery of steviol glycosides is summarized in FIG. 10. However, in accordance with embodiments of the present invention, the culture broth produced by the bioconversion strains will have a very high viscosity that presents challenges for removing the biomass and recovering steviol glycosides.

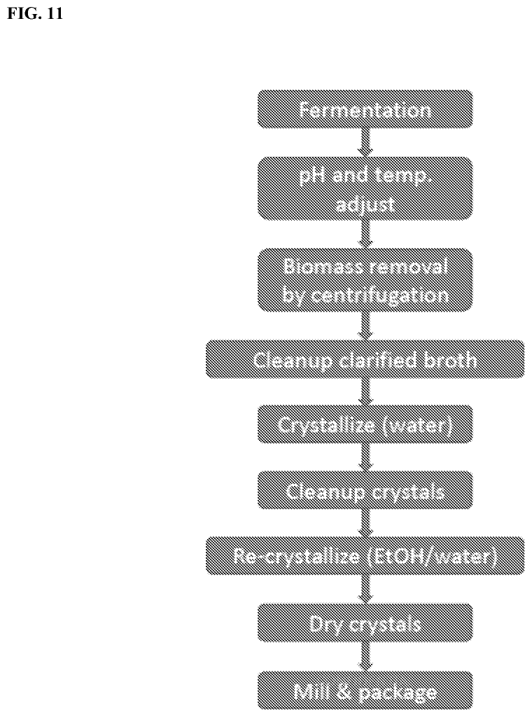

Temperature, pH adjustment, and/or addition of glycoside solubility enhancers, prior to biomass removal, substantially alleviates this difficulty. For example, these treatments can lower the viscosity of the culture material, allow for precipitation of proteins, as well as solubilization of glycosides to facilitate their subsequent separation and recovery from the biomass. The process as outlined in FIG. 11 can result in a final product with ≥95% steviol glycosides (e.g., RebM). For example, lowering pH (from the starting pH of about 7) to a pH in the range of about 2 to 4 enhances solubility of RebM considerably. Alternatively, increasing pH (from initial pH of about 7) to >pH 11 can also enhance solubility. With pH adjustment and solubility enhancers, it is possible to achieve concentrations of >100 g/L or >150 g/L of steviol glycosides with high purity in accordance with the current disclosure. Exemplary solubility enhancers include glycerol (e.g., about 0.5 wt %) and 1,3-propanediol (e.g., about 0.5 wt %). Other solubility enhancers include polyvinyl alcohol, polyethylene glycol, and polypropylene glycol, in addition to others described herein.

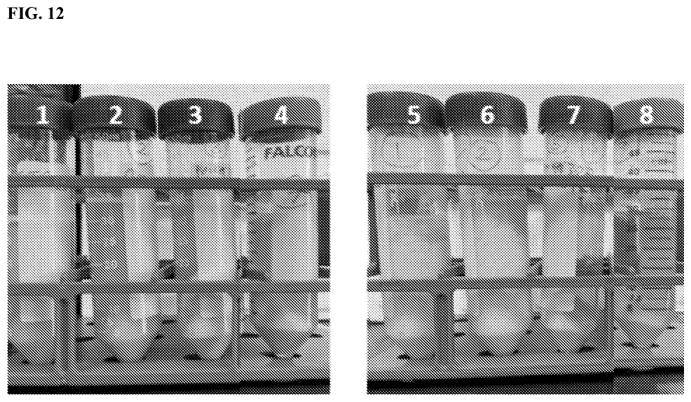

FIG. 12 shows the effect of various treatments on the separation of biomass from aqueous broth following centrifugation. The compactness of the pellet and clarity of supernatant serve as indicators of the ease of biomass removal. Briefly, 30 ml of fermentation broth was subjected to the following treatments, either singly or in combination: heating at 70° C. for 30 min, acidification to pH 3.78, or addition of 15% v/v ethanol. Broth was then centrifuged at 3000 rpm for 5 min to determine efficiency of separation. Results show a positive impact of heating and acidification on separation. Tube No. 8, which showed the best separation was heated to 70° C., and pH adjusted to 3.78. Tube No. 7, which included pH adjustment to 3.78 with addition of ethanol, also showed good separation.

Table 5 shows the effect of heating (70° C., 30 min) and acidification (pH 3.6) on processing time required for biomass removal. Briefly, treated and untreated fermentation broth were passed through a GEA Westfalia SB7 Separator to test removal of biomass. Untreated broth required three separate passes through the SB7 at a processing time of 0.44 min/L each followed by tangential flow filtration (TFF) at a processing time of 2.2 min/L to achieve sufficient separation of biomass for downstream steps. Treatment with heating and acidification enabled efficient biomass separation with just a single pass through the SB7, leading to a more than eight-fold faster processing time.

TABLE 5

| | | | | | Time required to |
|---|---|---|---|---|---|
| | 1st pass | 2nd pass | 3rd pass | TFF | remove biomass |
| Method | (min/L) | (min/L) | (min/L) | (min/L) | (min/L) |
| No treatment | 0.444 | 0.444 | 0.444 | 2.273 | 3.606 |
| pH + temp | 0.444 | — | — | — | 0.444 |

Table 6 shows the effect of heating and acidification on the quantification and recovery of RebM. Briefly, RebM concentrations in aqueous broth were measured before and after treatment with both heating (70° C., 15 min) and acidification (pH 2.5). Treatment resulted in a 120-173% improvement in RebM recovery compared to the untreated conditions (an average of 140% improvement over seven independent samples.)

TABLE 6

| Sample No. | 1 | 2 | 3 | 4 | 5 | 6 | 7 | Average |
|---|---|---|---|---|---|---|---|---|
| No treatment - RebM (g/L) | 8.32 | 16.49 | 15.55 | 15.37 | 15.99 | 12.31 | 22.28 | |
| pH + Temp - RebM (g/L) | 12.48 | 21.79 | 18.82 | 19.91 | 22.07 | 21.27 | 30.85 | |
| % RebM recovered | 150% | 132% | 121% | 130% | 138% | 173% | 138% | 140% |

Table 7 shows the effect of heating combined with acid or base addition and/or the addition of small molecule enhancers on the solubility of RebM. Fermentation broth was held at a constant temperature of 70° C. and subjected to different treatments as described in Table 7. RebM was slowly added with constant stirring to determine the maximum solubility of RebM. Acidification of the media showed an increase in RebM solubility, as did the addition of small molecule enhancers (glycerol, 1,3-propanediol). In some cases, the addition of enhancers plus a change in pH (base addition) resulted in further increases in RebM solubility.

TABLE 7

| | | | | RebM |
|---|---|---|---|---|
| | Temp. | Small | wt % | solubility |
| pH | (° C.) | molecule | enhancer | (g/L) |
| 7 | 70.8 | (none) | 0 | 29.9 |
| 3.5 | 70.8 | (none) | 0 | 68.9 |
| 7 | 70.8 | Glycerol | 0.5 | 119.0 |
| 11.6 | 70.8 | Glycerol | 0.5 | 151.3 |
| 11.6 | 70.8 | 1,3-Propanediol | 0.5 | 151.3 |

The process described here can produce a product with desirable qualities, including: ≥95% glycoside (e.g., RebM or mog.V) purity, attractive white color, easy solubilization, odorless, and high recovery yield. In particular, initial pH and temperature adjustment of the culture can change fluid characteristics of the broth, and increase efficiency of the disc stack separator for biomass removal. Further, solubility and therefore yield of glycosides is substantially increased by the pH and temperature adjustment (which avoids substantial losses of product in the solid phase).

The process may employ one or more crystallization steps. The crystallization process can include a static phase followed by a stirred phase, and optionally an evaporative phase, to control crystal morphology. The static phase can grow large crystals with a high degree of crystalline domains. Crystallization can include a process of seeding crystals, or use a system that does not involve seeding of crystals (i.e., crystals are spontaneously formed). For example, using a crystal seeding process, after seeding crystals during a static phase, a stirred phase will rapidly grow the crystals, and increase the degree of amorphous domains. Using this process, resulting crystals can have good final solubility. Resulting crystals will have a high purity of steviol glycoside (e.g., RebM), and will be easier to recover and wash. For recrystallization, an exemplary recrystallization solution system can comprise water, or in some embodiments includes ethanol (e.g., 1:2 EtOH:H₂O). In some embodiments, the recrystallization solution further comprises glycerol (e.g., up to 2%). The pH of the solution for recrystallization can vary, such as from about 4.0 to about 12.0.

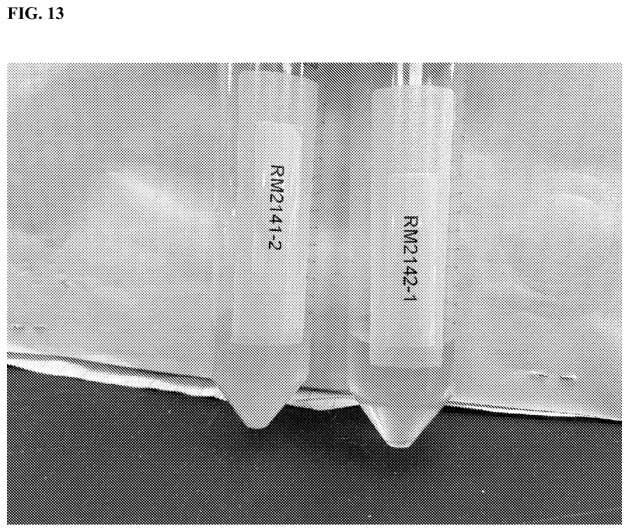

Prior to recrystallization, the solution can be filtered. Further, the selection of filter material can have a significant impact on the quality of the final product. For example, FIG. 13 compares the use of a hydrophobic filer material such as polypropylene (PP) and a relatively hydrophilic material such as polyethersulfone (PES) to filter the solution prior to recrystallization (both with 0.2-micron pore size). The RebM final product (>98%) is dissolved in propylene glycol to a concentration of 10 wt %. As shown in FIG. 13, the use of a PP filter results in a solution that is quite cloudy (left side) whereas the use of PES filter yields a solution that is clear (right side). Accordingly, PES filter materials, and similar hydrophilic materials provide significant advantages, likely by adsorption of impurities.

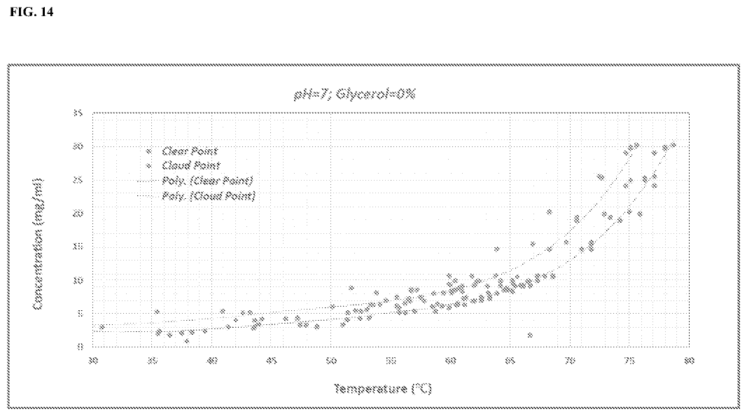

Understanding the thermodynamic and kinetic properties of crystallization systems is critical for the proper design of an industrial process. For example, the thermodynamic solubility represents the maximum concentration that a solute can reach (saturation or solubility) at a given temperature. The region above this solubility curve (see FIG. 14, for example) is supersaturated, and the region below it is undersaturated. For crystallization to proceed, a solution must be supersaturated. One way to achieve supersaturation is to reduce the temperature of a saturated solution. However, crystallization is not immediately initiated in this context; rather, the system enters a metastable zone of the supersaturated space that has a specified width. In this zone there is no spontaneous nucleation, but crystallization can be initiated by adding seed crystal. If no seed is added while cooling, the system will reach the edge of the metastable zone, past which point spontaneous nucleation will occur. The higher the degree of supersaturation the faster the nucleation rate will be, which has a significant impact on the particle size and crystal growth rate. As such, knowledge of the solubility and metastable zone width enables the design of an industrial crystallization process that has a desirable crystal size distribution (CSD) and also proceeds at a practical speed. In the case of unseeded crystallization, the edge of the metastable zone must be reached and exceeded slightly to initiate crystallization without the negative effects of too much spontaneous nucleation (e.g., which produces very small crystals). As the process proceeds, the concentration will drop back into the metastable zone after which supersaturation needs to be maintained (by cooling, evaporation, etc.) throughout the duration of the crystallization. In the case of seeded crystallization, it is important not to exceed the metastable zone to avoid spontaneous nucleation and seed must be added after the solution is saturated, so that seed crystal does not dissolve and does initiate nucleation. In both the seeded and unseeded cases, understanding the thermodynamics and kinetics of the system enables the design of the ramping rates (or evaporation rates) as well as nucleation points of the crystallization process.

Studies were undertaken to understand the solubility of RebM in different solvent systems, and to explore the effects of solubility enhances, to enable seeding and temperature ramp down strategies for crystal formation and growth. For these studies, Crystal 16 system (Crystalline Series) from Technobis Crystallization systems was used to generate Clear Points during an increasing temperature ramp (as a surrogate for solubility) and Cloud Points during a decreasing ramp (as a surrogate for the metastable zone width). Various solvent systems were evaluated.

TABLE 8
Experimental Design for Recrystallization Step

| | |
|---|---|
| Solvent Systems | 100% Water |
| | 67% Water/33% Ethanol |
| Solubility Temperature | 1. 5° C. to 85° C. at 0.5° C. per minute |
| Ramp Conditions | 2. Hold at 70° C. for 10 minutes |
| | 3. 85° C. to 5° C. at 0.2° C. per minute |
| Variables Tested | pH 7 or 11 |
| | Glycerol loading: 0% or 0.5% |

FIG. 14 shows the solubility (bottom curve) and metastable limit curve (top curve), defining the metastable zone width, as determined for RebM in water (pH 7.0, 0% glycerol), enabling control of crystal growth in this solvent system.

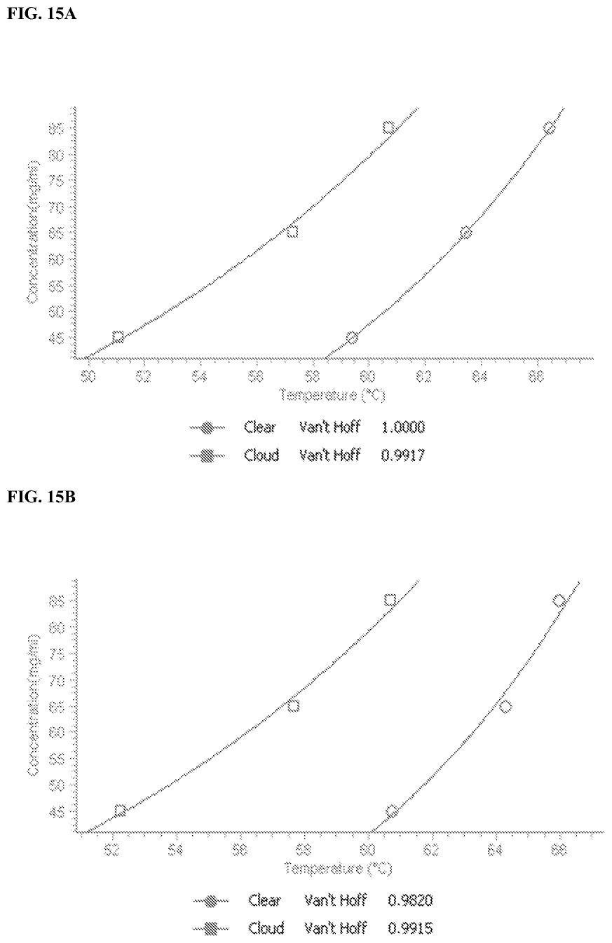

FIG. 15A, 15B show the solubility (bottom curve) and metastable limit curve (top curve), defining the metastable zone width, as determined for RebM in 67% water/33% ethanol at pH 7, enabling control of crystal growth in this solvent system. FIG. 15A is 0% glycerol, while FIG. 15B includes 0.5% glycerol.

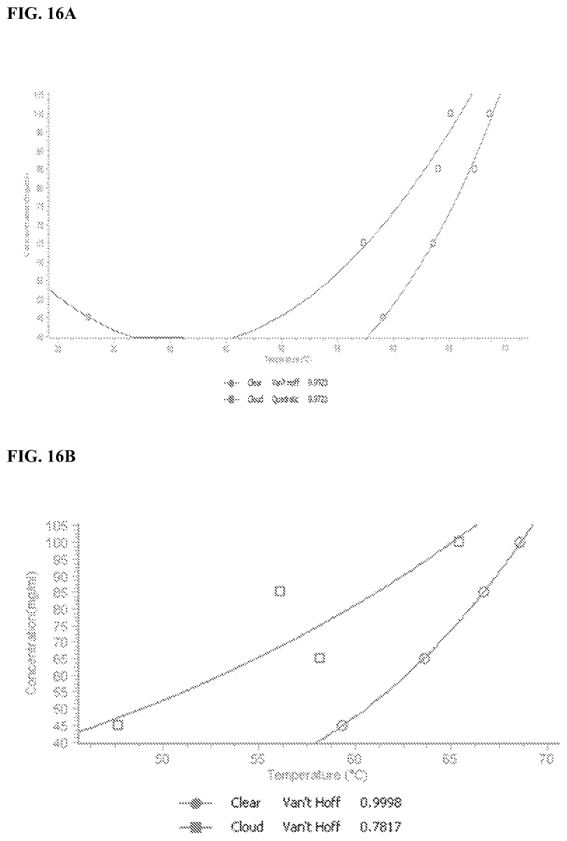

FIG. 16A, 16B show the solubility (bottom curve) and metastable limit curve (top curve), defining the metastable zone width, as determined for RebM in 67% water/33% ethanol at pH 11, enabling control of crystal growth in this solvent system. FIG. 16A is 0% glycerol, while FIG. 16B includes 0.5% glycerol.

In some embodiments, the recovery process will include one or more steps of tangential flow filtration (TFF). For example, TFF with a filter having a pore size of about 5 kD can remove endotoxin, large proteins, and other cell debris, while also enhancing solubility of the final powdered product. TFF with a filter having a pore size of about 0.5 kD can also be employed downstream to remove small molecule impurities and salts, and/or to concentrate the mother liquor for recrystallization.

Washing at basket centrifuge steps can employ washes with water, or alternatively other rinses can be employed. For example, chilled water/ethanol (e.g., 15% ethanol) can improve the quality of the cake. The mother liquor can be employed as wash water, or can be reworked.

Other processes that can be employed include activated carbon treatment, bentonite treatment, ion exchange chromatography, and concentration by evaporation. In particular, activated carbon treatment after dissolution of wet cake in EtOH:H₂O may improve the color of the final product.

### Example 4: Bioconversion of Mogrol or Mogrol Glycoside Precursors

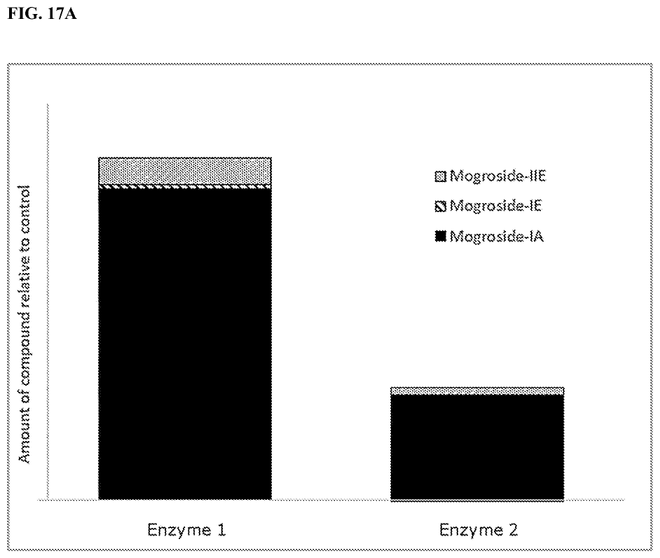

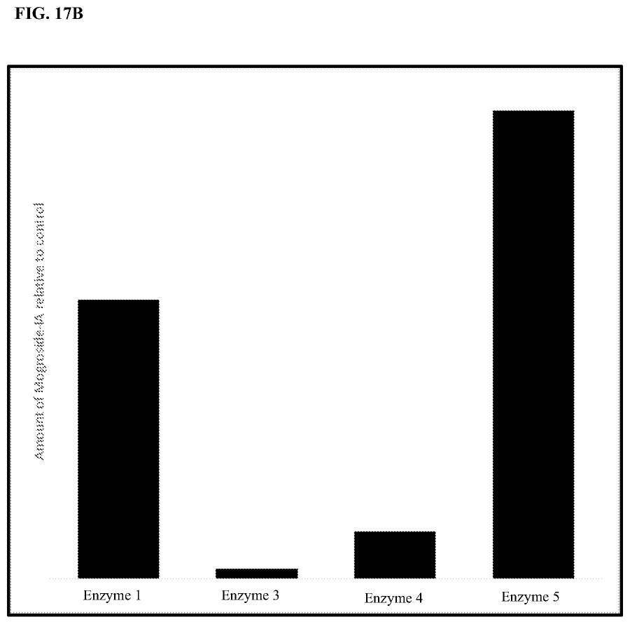

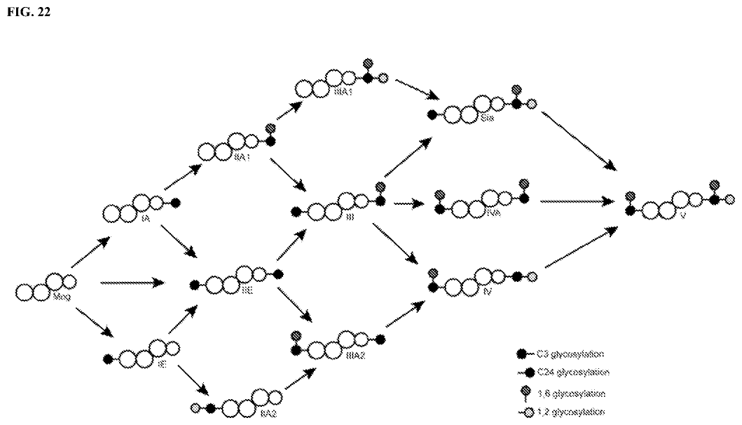

The engineered bacterial strains described herein can be used for the glycosylation of a variety of substrates, including but not limited to terpenoid glycosides. In some aspects, the invention identifies UGT enzymes that are active on the mogrol or mogroside scaffolds. Glycosylation pathways for the production of various mogrosides is provided in FIG. 22. FIGS. 17A and B show the bioconversion of mogrol into mogroside intermediates. Engineered E. coli strains (chassis strain) expressing UGT enzymes were incubated in 96-well plates with mogrol. Product formation was examined after 48 hours. Reported values are those in excess of the empty vector control. Products were measured on LC-MS/MS with authentic standards. As shown in FIG. 17A, both Enzyme 1 (SEQ ID NO: 71) and Enzyme 2 (SEQ ID NO: 33) UGT enzymes largely produced Mog.IA from mogrol, with a smaller amount of Mog.IE and/or Mog.IIE formed. FIG. 17B shows the bioconversion of mogrol into mogroside-IA using engineered E. coli strains expressing Enzyme 1 (SEQ ID NO: 71), Enzyme 3 (SEQ ID NO: 33), Enzyme 4 (SEQ ID NO: 82), and Enzyme 5 (SEQ ID NO: 83).

FIG. 18A and FIG. 18B shows the bioconversion of Mog. IA (FIG. 18A) or Mog. IE (FIG. 18B) into Mog. IE. In the experiment, engineered E. coli chassis strains expressing UGT enzymes, SEQ ID NO: 84, SEQ ID NO: 71, or SEQ ID NO: 33 were incubated in fermentation media containing Mog. IA (FIG. 18A) or Mog. IE (FIG. 18B) in 96-well plates at 37° C. Product formation was examined after 48 hours. Products were measured on LC-MS/MS with authentic standards. The values of Mog.IIE levels in excess of the empty vector control were calculated. As shown in FIG. 18A, SEQ ID NO: 84 and SEQ ID NO: 71 were able to catalyze bioconversion of Mog.IA into Mog.IIE. Similarly, as shown in FIG. 18B, SEQ ID NO: 84, SEQ ID NO: 71, and SEQ ID NO: 33 were able to catalyze the bioconversion of Mog.IE into Mog.IIE.

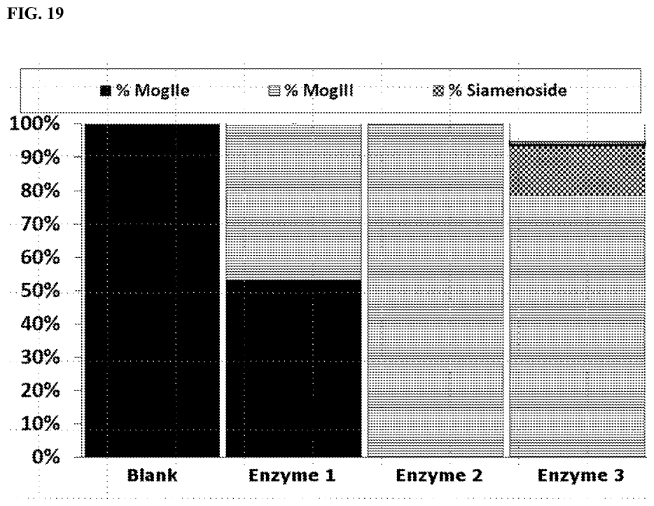

FIG. 19 shows the production of Mog.III or siamenoside from Mog.II-E. In the experiment, engineered E. coli strains expressing UGT enzymes SEQ ID NO: 72, SEQ ID NO: 54 or SEQ ID NO: 13 were grown in fermentation media containing Mog.II-E at 37° C. for 48 hr. Products were quantified by LCMS/MS with authentic standards of each compound. As shown in FIG. 19, all strains were able to catalyze bioconversion of Mog. IIE to Mog.III. In addition, the enzyme of SEQ ID NO: 13 also showed production of substantial amounts of siamenoside.

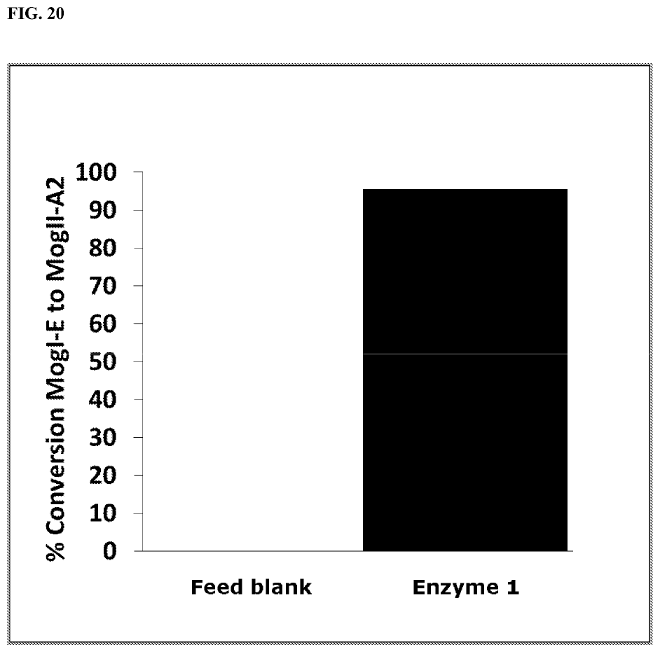

FIG. 20 shows the production of Mog. II-A2. Mog. I-E was fed in vitro. In the experiment, engineered E. coli strains (chassis strain) expressing UGT enzyme SEQ ID NO: 73 were incubated at 37° C. for 48 hr. Products were quantified by LC-MS/MS with authentic standards of each compound. As shown in FIG. 20, SEQ ID NO: 73 is able to catalyze bioconversion of Mog. IE to Mog. II-A2.

A summary of observed primary glycosylation reactions at C3 and C24 hydroxyls of mogrol are provided in Table 8. Specifically, mogrol was fed to cells expressing various UGT enzymes. Reactions were incubated at 37° C. for 48 hrs. Products were quantified by LCMS/MS with authentic standards of each compound.

TABLE 8

| UGT | C3 O-Glucosylation | C24 O-Glucosylation |
|---|---|---|
| SEQ ID NO: 84 | Yes | Yes |
| SEQ ID NO: 17 | No | Yes |
| SEQ ID NO: 80 | No | Yes |
| SEQ ID NO: 71 | Yes | Yes |
| SEQ ID NO: 46 | Yes | No |
| SEQ ID NO: 33 | Yes | Yes |
| SEQ ID NO: 83 | No | Yes |
| SEQ ID NO: 81 | No | Yes |
| SEQ ID NO: 82 | No | Yes |

A summary of branched glycosylation reactions are provided in Table 9. Mog. IIE or Mog. LE was fed to cells expressing various UGT enzymes. Reactions were incubated at 37° C. for 48 hr. Products were quantified by LC-MS/MS with authentic standards of each compound. “Indirect” evidence means that consumption of substrate was observed.

TABLE 9

| Name | C3 1-2 | C3 1-6 | C24 1-2 | C24 1-6 |
|---|---|---|---|---|
| SEQ ID NO: 73 | No | Yes | No | Yes |
| SEQ ID NO: 72 | No | Yes | No | No |
| SEQ ID NO: 39 | No | Yes | Yes | Yes |
| SEQ ID NO: 78 | No | No | Yes | No |
| SEQ ID NO: 54 | No | Yes | No | Yes |
| SEQ ID NO: 74 | No | Yes | No | Yes |
| SEQ ID NO: 76 | No | Yes | No | Yes |
| SEQ ID NO: 75 | Yes | Yes | Yes | Yes |
| | (Indirect) | | (Indirect) | (Indirect) |
| SEQ ID NO: 13 | Yes | Yes | Yes | Yes |
| | (Indirect) | (Indirect) | | |
| SEQ ID NO: 60 | No | Yes | Yes | Yes |
| SEQ ID NO: 77 | No | Yes | No | Yes |
| SEQ ID NO: 29 | No | No | No | Yes |
| SEQ ID NO: 79 | No | No | No | Yes |

Embodiments of the invention will now be defined with reference to the appended claims.

SEQUENCES
SUCROSE SYNTHASE ENZYMES
SEQ ID NO: 1: Solanum tuberosum (StSus1)
MAERVLTRVHSLRERVDATLAAHRNEILLFLSRIESHGKGILKPHELLAEFDAIRQDDKNKLNEHA
FEELLKSTQEAIVLPPWVALAIRLRPGVWEYIRVNVNALVVEELSVPEYLQFKEELVDGASNGNFV
LELDFEPFTASFPKPTLTKSIGNGVEFLNRHLSAKMFHDKESMTPLLEFLRAHHYKGKTMMLNDRI
QNSNTLQNVLRKAEEYLIMLPPETPYFEFEHKFQEIGLEKGWGDTAERVLEMVCMLLDLLEAPDSC
TLEKFLGRIPMVENVVILSPHGYFAQENVLGYPDTGGQVVYILDQVPALEREMLKRIKEQGLDIIP
RILIVTRLLPDAVGTTCGQRIEKVYGAEHSHILRVPFRTEKGIVRKWISRFEVWPYMETFIEDVAK
EISAELQAKPDLIIGNYSEGNLAASLLAHKLGVTQCTIAHALEKTKYPDSDIYWKKEDEKYHESSQ
FTADLIAMNHTDFIITSTFQEIAGSKDTVGQYESHMAFTMPGLYRVVHGINVEDPKFNIVSPGADI
NLYFSYSETEKRLTAFHPEIDELLYSDVENDEHLCVLKDRTKPILFTMARLDRVKNLTGLVEWYAK
NPRLRGLVNLVVVGGDRRKESKDLEEQAEMKKMYELIETHNLNGQFRWISSQMNRVRNGELYRYIA
DTKGAFVQPAFYEAFGLTVVEAMTCGLPTFATNHGGPAEIIVHGKSGFHIDPYHGEQAADLLADFF
EKCKKDPSHWETISMGGLKRIEEKYTWQIYSESLLTLAAVYGFWKHVSKLDRLEIRRYLEMFYALK
YRKMAEAVPLAAE
SEQ ID NO: 2: Solanum tuberosum (StSus2)
MAERVLTRVHSLRERLDATLAAHRNEILLELSRIESHGKGILKPHQLLAEFESIHKEDKDKLNDHA
FEEVLKSTQEAIVLPPWVALAIRLRPGVWEYVRVNVNALIVEELTVPEFLQFKEELVNGTSNDNEV
LELDFEPFTASFPKPTLTKSIGNGVEFLNRHLSAKMFHDKESMTPLLEFLRVHHYKGKTMMLNDRI
QNLYTLQKVLRKAEEYLTTLSPETSYSAFEHKFQEIGLERGWGDTAERVLEMICMLLDLLEAPDSC
TLEKFLGRIPMVFNVVILSPHGYFAQENVLGYPDTGGQVVYILDQVPALEREMLKRIKEQGLDIKP
RILIVTRLLPDAVGTTCGQRLEKVFGTEHSHILRVPFRTEKGIVRKWISRFEVWPYMETFIEDVGK
EITAELQAKPDLIIGNYSEGNLAASLLAHKLGVTQCTIAHALEKTKYPDSDIYLNKEDEKYHESAQ
FTADLIAMNHTDFIITSTFQEIAGSKDTVGQYESHMAFTMPGLYRVVHGIDVEDPKFNIVSPGADV
NLYFPYSEKEKRLTTFHPEIEDLLESDVENEEHLCVLKDRNKPIIFTMARLDRVKNLTGLVEWYAK
NPRLRELVNLVVVGGDRRKESKDLEEQAEMKKMYELIKTHNLNGQFRWISSQMNRVRNGELYRYIA
DTRGAFVQPAFYEAFGLTVVEAMSCGLPTFATNQGGPAEIIVHGKSGFQIDPYHGEQAADLLADFF
EKCKVDPSHWEAISEGGLKRIQEKYTWQIYSDRLLTLAAVYGFWKHVSKLDRLEIRRYLEMFYALK
FRKLAQLVPLAVE
SEQ ID NO: 3: Solanum tuberosum (StSus2 S11E)
MAERVLTRVHELRERLDATLAAHRNEILLELSRIESHGKGILKPHQLLAEFESIHKEDKDKLNDHA
FEEVLKSTQEAIVLPPWVALAIRLRPGVWEYVRVNVNALIVEELTVPEFLQFKEELVNGTSNDNEV
LELDFEPFTASFPKPTLTKSIGNGVEFLNRHLSAKMFHDKESMTPLLEFLRVHHYKGKTMMLNDRI
QNLYTLQKVLRKAEEYLTTLSPETSYSAFEHKFQEIGLERGWGDTAERVLEMICMLLDLLEAPDSC
TLEKFLGRIPMVFNVVILSPHGYFAQENVLGYPDTGGQVVYILDQVPALEREMLKRIKEQGLDIKP
RILIVTRLLPDAVGTTCGQRLEKVFGTEHSHILRVPFRTEKGIVRKWISRFEVWPYMETFIEDVGK
EITAELQAKPDLIIGNYSEGNLAASLLAHKLGVTQCTIAHALEKTKYPDSDIYLNKEDEKYHESAQ
FTADLIAMNHTDFIITSTFQEIAGSKDTVGQYESHMAFTMPGLYRVVHGIDVEDPKFNIVSPGADV
NLYFPYSEKEKRLTTFHPEIEDLLESDVENEEHLCVLKDRNKPIIFTMARLDRVKNLTGLVEWYAK
NPRLRELVNLVVVGGDRRKESKDLEEQAEMKKMYELIKTHNLNGQFRWISSQMNRVRNGELYRYIA
DTRGAFVQPAFYEAFGLTVVEAMSCGLPTFATNQGGPAEIIVHGKSGFQIDPYHGEQAADLLADFF
EKCKVDPSHWEAISEGGLKRIQEKYTWQIYSDRLLTLAAVYGFWKHVSKLDRLEIRRYLEMFYALK
FRKLAQLVPLAVE
SEQ ID NO: 4: Acidithiobacillus caldus (AcSuSy)
MAIEALRQQLLDDPRSWYAFLRHLVASQRDSWLYTDLQRACADFREQLPEGYAEGIGPLEDFVAHT
QEVIFRDPWMVFAWRPRPGRWIYVRIHREQLALEELSTDAYLQAKEGIVGLGAEGEAVLTVDERDE
RPVSRRLRDESTIGDGLTHLNRRLAGRIFSDLAAGRSQILEFLSLHRLDGQNLMLSNGNTDEDSLR
QTVQYLGTLPRETPWAEIREDMRRRGFAPGWGNTAGRVRETMRLLMDLLDSPSPAALESELDRIPM
ISRILIVSIHGWFAQDKVLGRPDTGGQVVYILDQARALEREMRNRLRQQGVDVEPRILIATRLIPE
SDGTTCDQRLEPVVGAENVQILRVPFRYPDGRIHPHWISRFKIWPWLERYAQDLEREVLAELGSRP
DLIIGNYSDGNLVATLLSERLGVTQCNIAHALEKSKYLYSDLHWRDHEQDHHFACQFTADLIAMNA
ADIIVTSTYQEIAGNDREIGQYEGHQDYTLPGLYRVENGIDVEDSKENIVSPGADPRFYESYARTE
ERPSFLEPEIESLLFGREPGADRRGVLEDRQKPLLLSMARMDRIKNLSGLAELYGRSSRLRGLANL
VIIGGHVDVGNSRDAEEREEIRRMHEIMDHYQLDGQLRWVGALLDKTVAGELYRVVADGRGVFVQP
ALFEAFGLTVIEAMSSGLPVFATRFGGPLEIIEDGVSGFHIDPNDHEATAERLADFLEAARERPKY
WLEISDAALARVAERYTWERYAERLMTIARIFGEWRFVLDRESQVMERYLQMFRHLQWRPLAHAVP
ME
SEQ ID NO: 5: Acidithiobacillus caldus (AcSuSy L637M-T640V)
MAIEALRQQLLDDPRSWYAFLRHLVASQRDSWLYTDLQRACADFREQLPEGYAEGIGPLEDEVAHT
QEVIFRDPWMVFAWRPRPGRWIYVRIHREQLALEELSTDAYLQAKEGIVGLGAEGEAVLTVDERDE
RPVSRRLRDESTIGDGLTHLNRRLAGRIFSDLAAGRSQILEFLSLHRLDGQNLMLSNGNTDEDSLR
QTVQYLGTLPRETPWAEIREDMRRRGFAPGWGNTAGRVRETMRLLMDLLDSPSPAALESELDRIPM
ISRILIVSIHGWFAQDKVLGRPDTGGQVVYILDQARALEREMRNRLRQQGVDVEPRILIATRLIPE
SDGTTCDQRLEPVVGAENVQILRVPFRYPDGRIHPHWISRFKIWPWLERYAQDLEREVLAELGSRP
DLIIGNYSDGNLVATLLSERLGVTQCNIAHALEKSKYLYSDLHWRDHEQDHHFACQFTADLIAMNA
ADIIVTSTYQEIAGNDREIGQYEGHQDYTLPGLYRVENGIDVEDSKFNIVSPGADPRFYESYARTE
ERPSFLEPEIESLLFGREPGADRRGVLEDRQKPLLLSMARMDRIKNLSGLAELYGRSSRLRGLANL
VIIGGHVDVGNSRDAEEREEIRRMHEIMDHYQLDGQLRWVGALMDKVVAGELYRVVADGRGVFVQP
ALFEAFGLTVIEAMSSGLPVFATREGGPLEIIEDGVSGFHIDPNDHEATAERLADFLEAARERPKY
WLEISDAALARVAERYTWERYAERLMTIARIFGFWRFVLDRESQVMERYLQMFRHLQWRPLAHAVP
ME
SEQ ID NO: 6: Arabidopsis thaliana (AtSus1)
MANAERMITRVHSQRERLNETLVSERNEVLALLSRVEAKGKGILQQNQIIAEFEALPEQTRKKLEG
GPFFDLLKSTQEAIVLPPWVALAVRPRPGVWEYLRVNLHALVVEELQPAEFLHFKEELVDGVKNGN
FTLELDFEPFNASIPRPTLHKYIGNGVDELNRHLSAKLFHDKESLLPLLKFLRLHSHQGKNLMLSE
KIQNLNTLQHTLRKAEEYLAELKSETLYEEFEAKFEEIGLERGWGDNAERVLDMIRLLLDLLEAPD
PCTLETFLGRVPMVFNVVILSPHGYFAQDNVLGYPDTGGQVVYILDQVRALEIEMLQRIKQQGLNI
KPRILILTRLLPDAVGTTCGERLERVYDSEYCDILRVPERTEKGIVRKWISRFEVWPYLETYTEDA
AVELSKELNGKPDLIIGNYSDGNLVASLLAHKLGVTQCTIAHALEKTKYPDSDIYWKKLDDKYHES
CQFTADIFAMNHTDFIITSTFQEIAGSKETVGQYESHTAFTLPGLYRVVHGIDVEDPKFNIVSPGA
DMSIYFPYTEEKRRLTKFHSEIEELLYSDVENKEHLCVLKDKKKPILFTMARLDRVKNLSGLVEWY
GKNTRLRELANLVVVGGDRRKESKDNEEKAEMKKMYDLIEEYKLNGQFRWISSQMDRVRNGELYRY
ICDTKGAFVQPALYEAFGLTVVEAMTCGLPTFATCKGGPAEIIVHGKSGFHIDPYHGDQAADTLAD
FFTKCKEDPSHWDEISKGGLQRIEEKYTWQIYSQRLLTLTGVYGFWKHVSNLDRLEARRYLEMFYA
LKYRPLAQAVPLAQDD
SEQ ID NO: 7: Arabidopsis thaliana (AtSus3)
MANPKLTRVLSTRDRVQDTLSAHRNELVALLSRYVDQGKGILQPHNLIDELESVIGDDETKKSLSD
GPFGEILKSAMEAIVVPPFVALAVRPRPGVWEYVRVNVFELSVEQLTVSEYLRFKEELVDGPNSDP
FCLELDFEPFNANVPRPSRSSSIGNGVQFLNRHLSSVMERNKDCLEPLLDELRVHKYKGHPLMLND
RIQSISRLQIQLSKAEDHISKLSQETPFSEFEYALQGMGFEKGWGDTAGRVLEMMHLLSDILQAPD
PSSLEKFLGMVPMVENVVILSPHGYFGQANVLGLPDTGGQVVYILDQVRALETEMLLRIKRQGLDI
SPSILIVTRLIPDAKGTTCNQRLERVSGTEHTHILRVPFRSEKGILRKWISREDVWPYLENYAQDA
ASEIVGELQGVPDFIIGNYSDGNLVASLMAHRMGVTQCTIAHALEKTKYPDSDIYWKDEDNKYHES
CQFTADLIAMNNADFIITSTYQEIAGTKNTVGQYESHGAFTLPGLYRVVHGIDVEDPKFNIVSPGA
DMTIYFPYSEETRRLTALHGSIEEMLYSPDQTDEHVGTLSDRSKPILESMARLDKVKNISGLVEMY
SKNTKLRELVNLVVIAGNIDVNKSKDREEIVEIEKMHNLMKNYKLDGQFRWITAQTNRARNGELYR
YIADTRGAFAQPAFYEAFGLTVVEAMTCGLPTFATCHGGPAEIIEHGLSGFHIDPYHPEQAGNIMA
DFFERCKEDPNHWKKVSDAGLQRIYERYTWKIYSERLMTLAGVYGFWKYVSKLERRETRRYLEMFY
ILKFRDLVKTVPSTADD
SEQ ID NO: 8: Vigna radiate (VrSS1)
MATDRLTRVHSLRERLDETLSANRNEILALLSRIEGKGKGILQHHQVIAEFEEIPEESRQKLTDGA
FGEVLRSTQEAIVLPPWVALAVRPRPGVWEYLRVNVHALVVEVLQPAEYLRFKEELVDGSSNGNFV
LELDFEPFTASFPRPTLNKSIGNGVQFLNRHLSAKLFHDKESLHPLLEFLRLHSVKGKTLMLNDRI
QNPDALQHVLRKAEEYLGTVPPETPYSAFEHKFQEIGLERGWGDNAERVLESIQLLLDLLEAPDPC
TLETFLGRIPMVFNVVILSPHGYFAQDNVLGYPDTGGQVVYILDQVRALENEMLHRIKQQGLDIVP
RILIITRLLPDAVGTTCGQRLEKVFGTEHSHILRVPERTENGIVRKWISRFEVWPYLETYTEDVAH
ELAKELQGKPDLIVGNYSDGNIVASLLAHKLGVTQCTIAHALEKTKYPESDIYWKKLEERYHESCQ
FTADLFAMNHTDFIITSTFQEIAGSKDTVGQYESHTAFTLPGLYRVVHGIDVEDPKFNIVSPGADQ
TIYFPHTETSRRLTSFHTEIEELLYSSVENEEHICVLKDRSKPIIFTMARLDRVKNITGLVEWYGK
NAKLRELVNLVVVAGDRRKESKDLEEKAEMKKMYSLIETYKLNGQFRWISSQMNRVRNGELYRVIA
DTKGAFVQPAVYEAFGLTVVEAMTCGLPTFATCNGGPAEIIVHGKSGFHIDPYHGDRAADLLVEFF
EKVKVDPSHWDKISQAGLQRIEEKYTWQIYSQRLLTLTGVYGFWKHVSNLDRRESRRYLEMFYALK
YRKLAESVPLAVE
SEQ ID NO: 9: Vigna radiate (VrSS1 S11E)
MATDRLTRVHELRERLDETLSANRNEILALLSRIEGKGKGILQHHQVIAEFEEIPEESRQKLTDGA
FGEVLRSTQEAIVLPPWVALAVRPRPGVWEYLRVNVHALVVEVLQPAEYLRFKEELVDGSSNGNFV
LELDFEPFTASFPRPTLNKSIGNGVQFLNRHLSAKLFHDKESLHPLLEFLRLHSVKGKTLMLNDRI
QNPDALQHVLRKAEEYLGTVPPETPYSAFEHKFQEIGLERGWGDNAERVLESIQLLLDLLEAPDPC
TLETFLGRIPMVFNVVILSPHGYFAQDNVLGYPDTGGQVVYILDQVRALENEMLHRIKQQGLDIVP
RILIITRLLPDAVGTTCGQRLEKVFGTEHSHILRVPERTENGIVRKWISRFEVWPYLETYTEDVAH
ELAKELQGKPDLIVGNYSDGNIVASLLAHKLGVTQCTIAHALEKTKYPESDIYWKKLEERYHESCQ
FTADLFAMNHTDFIITSTFQEIAGSKDTVGQYESHTAFTLPGLYRVVHGIDVEDPKFNIVSPGADQ
TIYFPHTETSRRLTSFHTEIEELLYSSVENEEHICVLKDRSKPIIFTMARLDRVKNITGLVEWYGK
NAKLRELVNLVVVAGDRRKESKDLEEKAEMKKMYSLIETYKLNGQFRWISSQMNRVRNGELYRVIA
DTKGAFVQPAVYEAFGLTVVEAMTCGLPTFATCNGGPAEIIVHGKSGFHIDPYHGDRAADLLVEFF
EKVKVDPSHWDKISQAGLQRIEEKYTWQIYSQRLLTLTGVYGFWKHVSNLDRRESRRYLEMFYALK
YRKLAESVPLAVE
SEQ ID NO: 10: Glycine Max (GmSS)
MATDRLTRVHSLRERLDETLTANRNEILALLSRIEAKGKGILQHHQVIAEFEEIPEENRQKLTDGA
FGEVLRSTQEAIVLPPWVALAVRPRPGVWEYLRVNVHALVVEELQPAEYLHFKEELVDGSSNGNFV
LELDFEPFNAAFPRPTLNKSIGNGVQFLNRHLSAKLFHDKESLHPLLEFLRLHSVKGKTLMLNDRI
QNPDALQHVLRKAEEYLGTVPPETPYSEFEHKFQEIGLERGWGDNAERVLESIQLLLDLLEAPDPC
TLETFLGRIPMVFNVVILSPHGYFAQDNVLGYPDTGGQVVYILDQVRALENEMLHRIKQQGLDIVP
RILIITRLLPDAVGTTCGQRLEKVFGTEHSHILRVPFRTEKGIVRKWISRFEVWPYLETYTEDVAH
ELAKELQGKPDLIVGNYSDGNIVASLLAHKLGVTQCTIAHALEKTKYPESDIYWKKLEERYHFSCQ
FTADLFAMNHTDFIITSTFQEIAGSKDTVGQYESHTAFTLPGLYRVVHGIDVEDPKFNIVSPGADQ
TIYFPHTETSRRLTSFHPEIEELLYSSVENEEHICVLKDRSKPIIFTMARLDRVKNITGLVEWYGK
NAKLRELVNLVVVAGDRRKESKDLEEKAEMKKMYGLIETYKLNGQFRWISSQMNRVRNGELYRVIC
DTRGAFVQPAVYEAFGLTVVEAMTCGLPTFATCNGGPAEIIVHGKSGFHIDPYHGDRAADLLVDFF
EKCKLDPTHWDKISKAGLQRIEEKYTWQIYSQRLLTLTGVYGFWKHVSNLDRRESRRYLEMFYALK
YRKLAESVPLAAE
SEQ ID NO: 11: Glycine Max (GmSS S11E)
MATDRLTRVHELRERLDETLTANRNEILALLSRIEAKGKGILQHHQVIAEFEEIPEENRQKLTDGA
FGEVLRSTQEAIVLPPWVALAVRPRPGVWEYLRVNVHALVVEELQPAEYLHFKEELVDGSSNGNFV
LELDFEPENAAFPRPTLNKSIGNGVQFLNRHLSAKLFHDKESLHPLLEFLRLHSVKGKTLMLNDRI
QNPDALQHVLRKAEEYLGTVPPETPYSEFEHKFQEIGLERGWGDNAERVLESIQLLLDLLEAPDPC
TLETFLGRIPMVFNVVILSPHGYFAQDNVLGYPDTGGQVVYILDQVRALENEMLHRIKQQGLDIVP
RILIITRLLPDAVGTTCGQRLEKVFGTEHSHILRVPERTEKGIVRKWISRFEVWPYLETYTEDVAH
ELAKELQGKPDLIVGNYSDGNIVASLLAHKLGVTQCTIAHALEKTKYPESDIYWKKLEERYHESCQ
FTADLFAMNHTDFIITSTFQEIAGSKDTVGQYESHTAFTLPGLYRVVHGIDVEDPKFNIVSPGADQ
TIYFPHTETSRRLTSFHPEIEELLYSSVENEEHICVLKDRSKPIIFTMARLDRVKNITGLVEWYGK
NAKLRELVNLVVVAGDRRKESKDLEEKAEMKKMYGLIETYKLNGQFRWISSQMNRVRNGELYRVIC
DTRGAFVQPAVYEAFGLTVVEAMTCGLPTFATCNGGPAEIIVHGKSGFHIDPYHGDRAADLLVDFF
EKCKLDPTHWDKISKAGLQRIEEKYTWQIYSQRLLTLTGVYGFWKHVSNLDRRESRRYLEMFYALK
YRKLAESVPLAAE
SEQ ID NO: 12: Anabaena sp. (AsSusA)
MASELMQAILDSEEKHDLRGFISELRQQDKNYLLRNDILNVYAEYCSKCQKPETSYKESNLSKLIY
YTQEIIPEDSNFCFIIRPKIAAQEVYRLTADLDVEPMTVQELLDLRDRLVNKFHPYEGDILELDFG
PFYDYTPTIRDPKNIGKGVQYLNRYLSSKLFQDSQQWLESLENFLRLHNYNGIQLLINHQIQSQQQ
LSQQVKNALNFVSDRPNDEPYEQFRLQLQTMGFEPGWGNTASRVRDTLNILDELIDSPDPQTLEAF
ISRIPMIFRIVLVSAHGWFGQEGVLGRPDTGGQVVYVLDQAKNLEKQLQEDAILAGLEVLNVQPKV
IILTRLIPNSDGTLCNQRLEKVYGTENAWILRVPLREFNPKMTQNWISRFEFWPYLETFAIDSERE
LLAEFQGRPDLIVGNYTDGNLVAFLLTRRMKVTQCNIAHALEKSKYLESNLYWQDLEEKYHESLQF
TADLIAMNAANFVISSTYQEIVGTPDSIGQYESYKCFTMPELYHVVNGIELFSPKENVVPPGVNEN
SYFPYTQTQNRIESDRDRLEEMLFTLEDSSQIFGKLDDPNKRPIFSMARLDRIKNLTGLAECFGQS
QELQERCNLILVAGKLRIEESEDNEEKDEIVKLYRIIDEYNLHGKIRWLGVRLSKNDSGEIYRVIC
DRQGIFVQPALFEAFGLTILESMISGLPTFATQFGGPLEIIQDKINGFYINPTHLEETATKILDEV
TKCEQNPNYWNIISEKAIDRVYSTYTWKIHTTKLLTLARIYGEWNFTSKEKREDLLRYLESLFYLI
YKPRAQQLLEQHKYR
URIDINE DIPHOSPHATE-DEPENDENT GLYCOSYLTRANSFERASE (UGTs)
SEQ ID NO: 13: Synthetic (MbUGT1, 2.2)
MATKGSSGMSLAERFWLTLSRSSLVVGRSCVEFEPETVPLLSTLRGKPITFLGLMPPLHEGRREDG
EDATVRWLDAQPAKSVVYVALGSEVPLGVEKVHELALGLELAGTRELWALRKPTGVSDADLLPAGE
EERTRGRGVVATRWVPQMSILAHAAVGAFLTHCGWNSTIEGLMEGHPLIMLPIFGDQGPNARLIEA
KNAGLQVARNDGDGSEDREGVAAAIRAVAVEEESSKVFQAKAKKLQEIVADMACHERYIDGFIQQL
RSYKDDSGYSSSYAAAAGMHVVICPWLAFGHLLPCLDLAQRLASRGHRVSFVSTPRNISRLPPVRP
ALAPLVAFVALPLPRVEGLPDGAESTNDVPHDRPDMVELHRRAFDGLAAPFSEFLGTACADWVIVD
VFHHWAAAAALEHKVPCAMMLLGSAEMIASIADERLEHAETESPAAAGQGRPAAAPTFEVARMKLI
R
SEQ ID NO: 14: Synthetic (MbUGT1, 2.3)
MATKNSSGMSLAERFWLTLSRSSLVVGRSCVEFEPETVPLLSTLRGKPITELGLMPPLHEGRREDG
EDATVRWLDAQPAKSVVYVALGSEVPLGVEKVHELALGLELAGTRELWALRKPTGVSDADLLPAGE
EERTRGRGVVATRWVPQMSILAHAAVGAFLTHCGWNSTIEGLMEGHPLIMLPITGDQGPNARLIEA
KNAGLQVARNDGDGSFDREGVAAAIRAVAVEEESSKVFQAKAKKLQEIVADMACHERYIDGFIQQL
RSYKDDSGYSSSYAAAAGMHVVICPWLAFGHLLPCLDLAQRLASRGHRVSFVSTPRNISRLPPVRP
ALAPLVAFVALPLPRVEGLPDGAESTNDVPHDRPDMVELHRRAFDGLAAPFSEFLGTACADWVIVD
SFHHWAAAAALEHKVPCAMMLLGSAEMIASIADERLEHAETESPAAAGQGRPAAAPTFEVARMKLI
R
SEQ ID NO: 15: Synthetic (MbUGT1, 2.4)
MARRTKNSSGMSLAERFWLTLSRSSLVVGRSCVEFEPETVPLLSTLRGKPITELGLMPPLPEGRRE
DGEDATVRWLDAQPAKSVVYVALGSEVPLGVEKVHELALGLELAGTRELWALRKPTGVSDADLLPA
GFEERTRGRGVVATRWVPQMSILAHAAVGAFLTHCGWNSTIEGLMEGHPLIMLPITGDQGPNARLI
EAKNAGLQVARNDGDGSEDREGVAAAIRAVAVEEESSKVFQEKAKKLQEIVADMACHERYIDGFIQ
QLRSYKDDSGYSSSYAAAAGMHVVICPWLAFGHLLPCLDLAQRLASRGHRVSFVSTPRNISRLPPV
RPALAPLVAFVALPLPRVEGLPDGAESTNDVPHDRPDMVELHRRAFDGLAAPFSEFLGTACADWVI
VDSFHHWAAAAALEHKVPCAMMELGSAEMIASIADERLEHAETESPAAAGQGRPAAAPTFEVARMK
LIR
SEQ ID NO: 16: Synthetic (MbUGT1, 2.5)
MARRTKNSSGMSLAERFWLTLSRSSLVVGRSCVEFEPETVPLLSTLRGKPITELGLMPPLPEGRRE
DGEDATVRWLDAQPAKSVVYVALGSEVPLGVEKVHELALGLELAGTRELWALRKPTGVSDADLLPA
GFEERTRGRGVVATRWVPQAAILAHAAVGAFLTHCGWNSTIEGLMEGHPLIMLPITGDQGPNARLI
EAKNAGLQVARNDGDGSEDREGVAAAIRAVAVEEESSKVFQEKAKKLQEIVADMACHERYIDGFIQ
QLRSYKDDSGYSSSYAAAAGMHVVICPWLAFGHLLPCLDLAQRLASRGHRVSFVSTPRNISRLPPV
RPALAPLVAFVALPLPRVEGLPDGAESTNDVPHDRPDMVELHRRAFDGLAAPFSEFLGTACADWVI
VDSFHHWAAAAALEHKVPCAMMELGSAEMIASIADERLEHAETESPAAAGQGRPAAAPTFEVARMK
LIR
SEQ ID NO: 17: SrUGT85C2 (Stevia rebaudiana)
MDAMATTEKKPHVIFIPFPAQSHIKAMLKLAQLLHHKGLQITFVNTDFIHNQFLESSGPHCLDGAP
GFRFETIPDGVSHSPEASIPIRESLLRSIETNFLDRFIDLVTKLPDPPTCIISDGELSVETIDAAK
KLGIPVMMYWTLAACGFMGFYHIHSLIEKGFAPLKDASYLINGYLDTVIDWVPGMEGIRLKDEPLD
WSTDLNDKVLMFTTEAPQRSHKVSHHIFHTFDELEPSIIKTLSLRYNHIYTIGPLQLLLDQIPEEK
KQTGITSLHGYSLVKEEPECFQWLQSKEPNSVVYVNFGSTTVMSLEDMTEFGWGLANSNHYFLWII
RSNLVIGENAVLPPELEEHIKKRGFIASWCSQEKVLKHPSVGGFLTHCGWGSTIESLSAGVPMICW
PYSWDQLTNCRYICKEWEVGLEMGTKVKRDEVKRLVQELMGEGGHKMRNKAKDWKEKARIAIAPNG
SSSLNIDKMVKEITVLARN
SEQ ID NO: 18: SrUGT74G1 (Stevia rebaudiana)
MAEQQKIKKSPHVLLIPFPLQGHINPFIQFGKRLISKGVKTTLVTTIHTLNSTLNHSNTTTTSIEI
QAISDGCDEGGFMSAGESYLETFKQVGSKSLADLIKKLQSEGTTIDAIIYDSMTEWVLDVAIEFGI
DGGSFFTQACVVNSLYYHVHKGLISLPLGETVSVPGFPVLQRWETPLILQNHEQIQSPWSQMLEGQ
FANIDQARWVFTNSFYKLEEEVIEWTRKIWNLKVIGPTLPSMYLDKRLDDDKDNGENLYKANHHEC
MNWLDDKPKESVVYVAFGSLVKHGPEQVEEITRALIDSDVNFLWVIKHKEEGKLPENLSEVIKTGK
GLIVAWCKQLDVLAHESVGCFVTHCGENSTLEAISLGVPVVAMPQFSDQTTNAKLLDEILGVGVRV
KADENGIVRRGNLASCIKMIMEEERGVIIRKNAVKWKDLAKVAVHEGGSSDNDIVEFVSELIKA
SEQ ID NO: 19: SrUGT76G1 (Stevia rebaudiana)
MENKTETTVRRRRRIILFPVPFQGHINPILQLANVLYSKGESITIFHTNENKPKTSNYPHFTERFI
LDNDPQDERISNLPTHGPLAGMRIPIINEHGADELRRELELLMLASEEDEEVSCLITDALWYFAQS
VADSLNLRRLVLMTSSLENFHAHVSLPQFDELGYLDPDDKTRLEEQASGEPMLKVKDIKSAYSNWQ
ILKEILGKMIKQTKASSGVIWNSFKELEESELETVIREIPAPSFLIPLPKHLTASSSSLLDHDRTV
FQWLDQQPPSSVLYVSFGSTSEVDEKDELEIARGLVDSKQSFLWVVRPGFVKGSTWVEPLPDGELG
ERGRIVKWVPQQEVLAHGAIGAFWTHSGWNSTLESVCEGVPMIFSDFGLDQPLNARYMSDVLKVGV
YLENGWERGEIANAIRRVMVDEEGEYIRQNARVLKQKADVSLMKGGSSYESLESLVSYISSL
SEQ ID NO: 20: Synthetic (MbUGT1-3)
MANWQILKEILGKMIKQTKASSGVIWNSFKELEESELETVIREIPAPSFLIPLPKHLTASSSSLLD
HDRTVFQWLDQQPPSSVLYVSFGSTSEVDEKDFLEIARGLVDSKQSFLWVVRPGFVKGSTWVEPLP
DGFLGERGRIVKWVPQQEVLAHGAIGAFWTHSGWNSTLESVCEGVPMIFSDEGLDQPLNARYMSDV
LKVGVYLENGWERGEIANAIRRVMVDEEGEYIRQNARVLKQKADVSLMKGGSSYESLESLVSYISS
LENKTETTVRRRRRIILFPVPFQGHINPILQLANVLYSKGFSITIFHTNENKPKTSNYPHFTERFI
LDNDPQDERISNLPTHGPLAGMRIPIINEHGADELRRELELLMLASEEDEEVSCLITDALWYFAQS
VADSLNLRRLVLMTSSLENFHAHVSLPQFDELGYLDPDDKTRLEEQASGFPMLKVKDIKSAYS
SEQ ID NO: 21: UGT76G1 L200A (Stevia rebaudiana, L200A)
MAENKTETTVRRRRRIILFPVPFQGHINPILQLANVLYSKGFSITIFHTNENKPKTSNYPHFTFRE
ILDNDPQDERISNLPTHGPLAGMRIPIINEHGADELRRELELLMLASEEDEEVSCLITDALWYFAQ
SVADSLNLRRLVLMTSSLENFHAHVSLPQFDELGYLDPDDKTRLEEQASGEPMLKVKDIKSAYSNW
QIAKEILGKMIKQTKASSGVIWNSFKELEESELETVIREIPAPSFLIPLPKHLTASSSSLLDHDRT
VFQWLDQQPPSSVLYVSFGSTSEVDEKDFLEIARGLVDSKQSFLWVVRPGFVKGSTWVEPLPDGEL
GERGRIVKWVPQQEVLAHGAIGAFWTHSGWNSTLESVCEGVPMIFSDEGLDQPLNARYMSDVLKVG
VYLENGWERGEIANAIRRVMVDEEGEYIRQNARVLKQKADVSLMKGGSSYESLESLVSYISSL
SEQ ID NO: 22: Synthetic (MbUGT1-3 0)
MAKQSFLWVVRPGFVKGSTWVEPLPDGFELGERGRIVKWVPQQEVLAHGAIGAFWTHSGWNSTLESV
CEGVPMIFSDFGLDQPLNARYMSDVLKVGVYLENGWERGEIANAIRRVMVDEEGEYIRQNARVLKQ
KADVSLMKGGSSYESLESLVSYISSLENKTETTVRRRRRIILFPVPFQGHINPILQLANVLYSKGF
SITIFHTNENKPKTSNYPHFTFRFILDNDPQDERISNLPTHGPLAGMRIPIINEHGADELRRELEL
LMLASEEDEEVSCLITDALWYFAQSVADSLNLRRLVLMTSSLENFHAHVSLPQFDELGYLDPDDKT
RLEEQASGFPMLKVKDIKSAYSNWQIAKEILGKMIKQTKASSGVIWNSFKELEESELETVIREIPA
PSFLIPLPKHLTASSSSLLDHDRTVFQWLDQQPPSSVLYVSFGSTSEVDEKDELEIARGLVDS
SEQ ID NO: 23: Synthetic (MbUGT1-3 1)
MAFLWVVRPGFVKGSTWVEPLPDGELGERGRIVKWVPQQEVLAHGAIGAFWTHGGWNSTLESVCEG
VPMIFSDFGLDQPLNARYMSDVLKVGVYLENGWERGEIANAIRRLMVDEEGEYIRQNARVLKQKAD
VSLMKGGSSYESLESLVSYISSLGSGGSGGSGRRRRIILFPVPFQGHINPMLQLANVLYSKGESIT
IFHTNFNKPKTSNYPHFTFRFILDNDPQDERISNLPTHGPLAGMRIPIINEHGADELRRELELLML
ASEEDEEVSCLITDALWYFAQSVADSLNLRRLVLMTSSLENFHAHVSLPQFDELGYLDPDDKTRLE
EQASGFPMLKVKDIKSAYSNWQIAKEILGKMIKQTKASSGVIWNSFKELEESELETVIREIPAPSF
LIPLPKHLTASSSSLLDHDRTVFQWLDQQPPSSVLYVSFGSTSEVDEKDFLEIARGLVDSQS
SEQ ID NO: 24: Synthetic (MbUGT1-3 2)
MAFLWVVRPGFVKGSTWVEPLPDGELGERGRIVKWVPQQEVLAHGAIGAFWTHGGWNSTLESVCEG
VPMIFSDFGLDQPLNARYMSDVLKVGVYLENGWERGEIANAIRRLMVDEEGEYIRQNARVLKQKAD
VSLMKGGSSYESLESLVSYISSLGSGGSGRRRRIILFPVPFQGHINPMLQLANVLYSKGFSITIFH
TNFNKPKTSNYPHFTERFILDNDPQDERISNLPTHGPLAGMRIPIINEHGADELRRELELQMLASE
EDEEVSCLITDALWYFAQSVADSLNLPRLVLMTSSLENFHAHVSLPQFDELGYLDPDDKTRLEEQA
SGFPMLKVKDIKSAYSNWQIAKEILGKMIKQTKASSGVIWNSFKELEESELETVIREIPAPSFLIP
LPKHLTASSSSLLEHDRTVFQWLDQQPPSSVLYVSFGSTSEVDEKDFLEIARGLVDSQS
SEQ ID NO: 25: Synthetic (MbUGT1-3 3)
MAFLWVVRPGFVKGSTWVEPLPDGFLGERGRIVKWVPQQEVLAHGAIGAFWTHGGWNSTLESVCEG
VPMIFQDFGLDQPLNARYMSDVLKVGVYLENGWERGEIANAIRRLMVDEEGEYIRQNARVLKQKAD
VSLMKGGSSYESLESLVSYISSLGSGGSGRRRRIILFPVPFQGHINPMLQLANVLYSKGESITIFH
TNFNKPKTSNYPHFTFRFILDNDPQDHGPLAGMRIPIINEHGADELRRELELQMLASEEDEEVSCL
ITDALWYFAQSVADSLNLPRLVLMTSSLENFHCHVSLPQFDELGYLDPDDKTRLEEQASGFPMLKV
KDIKSAFSNWQIAKEILGKMIKQTKASSGVIWNSFKELEESELETVIREIPAPSFLIPLPKHLTAS
SSSLLEHDRTVFQWLDQQPPSSVIYVSFGSTSEVDEKDFLEIARGLVDSQS
SEQ ID NO: 26: SrUGT91D1 (Stevia rebaudiana)
MYNVTYHQNSKAMATSDSIVDDRKQLHVATFPWLAFGHILPFLQLSKLIAEKGHKVSFLSTTRNIQ
RLSSHISPLINVVQLTLPRVQELPEDAEATTDVHPEDIQYLKKAVDGLQPEVTRELEQHSPDWIIY
DFTHYWLPSIAASLGISRAYFCVITPWTIAYLAPSSDAMINDSDGRTTVEDLTTPPKWFPFPTKVC
WRKHDLARMEPYEAPGISDGYRMGMVFKGSDCLLFKCYHEFGTQWLPLLETLHQVPVVPVGLLPPE
IPGDEKDETWVSIKKWLDGKQKGSVVYVALGSEALVSQTEVVELALGLELSGLPFVWAYRKPKGPA
KSDSVELPDGFVERTRDRGLVWTSWAPQLRILSHESVCGELTHCGSGSIVEGLMEGHPLIMLPIFC
DQPLNARLLEDKQVGIEIPRNEEDGCLTKESVARSLRSVVVENEGEIYKANARALSKIYNDTKVEK
EYVSQFVDYLEKNARAVAIDHES
SEQ ID NO: 27: SrUGT91D2 (Stevia rebaudiana)
MATSDSIVDDRKQLHVATFPWLAFGHILPYLQLSKLIAEKGHKVSELSTTRNIQRLSSHISPLINV
VQLTLPRVQELPEDAEATTDVHPEDIPYLKKASDGLQPEVTRFLEQHSPDWIIYDYTHYWLPSIAA
SLGISRAHFSVTTPWAIAYMGPSADAMINGSDGRTTVEDLTTPPKWFPFPTKVCWRKHDLARLVPY
KAPGISDGYRMGLVLKGSDCLLSKCYHEFGTQWLPLLETLHQVPVVPVGLLPPEVPGDEKDETWVS
IKKWLDGKQKGSVVYVALGSEVLVSQTEVVELALGLELSGLPFVWAYRKPKGPAKSDSVELPDGFV
ERTRDRGLVWTSWAPQLRILSHESVCGFLTHCGSGSIVEGLMFGHPLIMLPIFGDQPLNARLLEDK
QVGIEIPRNEEDGCLTKESVARSLRSVVVEKEGEIYKANARELSKIYNDTKVEKEYVSQFVDYLEK
NTRAVAIDHES
SEQ ID NO: 28: SrUGT91D2e (Stevia rebaudiana)
MATSDSIVDDRKQLHVATFPWLAFGHILPYLQLSKLIAEKGHKVSELSTTRNIQRLSSHISPLINV
VQLTLPRVQELPEDAEATTDVHPEDIPYLKKASDGLQPEVTRFLEQHSPDWIIYDYTHYWLPSIAA
SLGISRAHFSVTTPWAIAYMGPSADAMINGSDGRTTVEDLTTPPKWFPFPTKVCWRKHDLARLVPY
KAPGISDGYRMGLVLKGSDCLLSKCYHEFGTQWLPLLETLHQVPVVPVGLLPPEIPGDEKDETWVS
IKKWLDGKQKGSVVYVALGSEVLVSQTEVVELALGLELSGLPFVWAYRKPKGPAKSDSVELPDGFV
ERTRDRGLVWTSWAPQLRILSHESVCGFLTHCGSGSIVEGLMEGHPLIMLPIFGDQPLNARLLEDK
QVGIEIPRNEEDGCLTKESVARSLRSVVVEKEGEIYKANARELSKIYNDTKVEKEYVSQFVDYLEK
NARAVAIDHES
SEQ ID NO: 29: OsUGT1-2 (Oryza sativa)
MDSGYSSSYAAAAGMHVVICPWLAFGHLLPCLDLAQRLASRGHRVSFVSTPRNISRLPPVRPALAP
LVAFVALPLPRVEGLPDGAESTNDVPHDRPDMVELHRRAFDGLAAPFSEFLGTACADWVIVDVFHH
WAAAAALEHKVPCAMMLLGSAHMIASIADRRLERAETESPAAAGQGRPAAAPTFEVARMKLIRTKG
SSGMSLAERFSLTLSRSSLVVGRSCVEFEPETVPLLSTLRGKPITFLGLMPPLHEGRREDGEDATV
RWLDAQPAKSVVYVALGSEVPLGVEKVHELALGLELAGTRELWALRKPTGVSDADLLPAGFEERTR
GRGVVATRWVPQMSILAHAAVGAFLTHCGWNSTIEGLMEGHPLIMLPIFGDQGPNARLIEAKNAGL
QVARNDGDGSFDREGVAAAIRAVAVEEESSKVFQAKAKKLQEIVADMACHERYIDGFIQQLRSYKD
SEQ ID NO: 30: Synthetic (MbUGTC19)
MAECMNWLDDKPKESVVYVAFGSLVKHGPEQVEEITRALIDSDVNFLWVIKHKEEGKLPENLSEVI
KTGKGLIVAWCKQLDVLAHESVGCFVTHCGENSTLEAISLGVPVVAMPQFSDQTTNAKLLDEILGV
GVRVKADENGIVRRGNLASCIKMIMEEERGVIIRKNAVKWKDLAKVAVHEGGSSDNDIVEFVSELI
KAGSGEQQKIKKSPHVLLIPFPLQGHINPFIQFGKRLISKGVKTTLVTTIHTLNSTLNHSNTTTTS
IEIQAISDGCDEGGEMSAGESYLETFKQVGSKSLADLIKKLQSEGTTIDAIIYDSMTEWVLDVAIE
FGIDGGSFFTQACVVNSLYYHVHKGLISLPLGETVSVPGFPVLQRWETPLILQNHEQIQSPWSQML
FGQFANIDQARWVFTNSFYKLEEEVIEWTRKIWNLKVIGPTLPSMYLDKRLDDDKDNGENLYKANH
H
SEQ ID NO: 31: Synthetic (MbUGTC19-2)
MANHHECMNWLDDKPKESVVYVAFGSLVKHGPEQVEEITRALIDSDVNELWVIKHKEEGKLPENLS
EVIKTGKGLIVAWCKQLDVLAHESVGCFVTHCGENSTLEAISLGVPVVAMPQFSDQTTNAKLLDEI
LGVGVRVKADENGIVRRGNLASCIKMIMEEERGVIIRKNAVKWKDLAKVAVHEGGSSDNDIVEFVS
ELIKAGSGEQQKIKKSPHVLLIPFPLQGHINPFIQFGKRLISKGVKTTLVTTIHTLNSTLNHSNTT
TTSIEIQAISDGCDEGGEMSAGESYLETFKQVGSKSLADLIKKLQSEGTTIDAIIYDSMTEWVLDV
AIEFGIDGGSFFTQACVVNSLYYHVHKGLISLPLGETVSVPGFPVLQRWETPLILQNHEQIQSPWS
QMLFGQFANIDQARWVFTNSFYKLEEEVIEWTRKIWNLKVIGPTLPSMYLDKRLDDDKDNGENLYK
A
SEQ ID NO: 32: MbUGTC13 (Stevia rebaudiana UGT85C2, P215T)
MADAMATTEKKPHVIFIPFPAQSHIKAMLKLAQLLHHKGLQITFVNTDFIHNQFLESSGPHCLDGA
PGFRFETIPDGVSHSPEASIPIRESLLRSIETNELDRFIDLVTKLPDPPTCIISDGELSVETIDAA
KKLGIPVMMYWTLAACGFMGFYHIHSLIEKGFAPLKDASYLTNGYLDTVIDWVPGMEGIRLKDEPL
DWSTDLNDKVLMFTTEATQRSHKVSHHIFHTEDELEPSIIKTLSLRYNHIYTIGPLQLLLDQIPEE
KKQTGITSLHGYSLVKEEPECFQWLQSKEPNSVVYVNFGSTTVMSLEDMTEFGWGLANSNHYELWI
IRSNLVIGENAVLPPELEEHIKKRGFIASWCSQEKVLKHPSVGGFLTHCGWGSTIESLSAGVPMIC
WPYSWDQLTNCRYICKEWEVGLEMGTKVKRDEVKRLVQELMGEGGHKMRNKAKDWKEKARIAIAPN
GSSSLNIDKMVKEITVLARN
SEQ ID NO: 33: SgUGT720-269-1 (Siraitia grosvenorii)
MEDRNAMDMSRIKYRPQPLRPASMVQPRVLLFPFPALGHVKPFLSLAELLSDAGIDVVELSTEYNH
RRISNTEALASRFPTLHFETIPDGLPPNESRALADGPLYFSMREGTKPRFRQLIQSLNDGRWPITC
IITDIMLSSPIEVAEEFGIPVIAFCPCSARYLSIHFFIPKLVEEGQIPYADDDPIGEIQGVPLFEG
LLRRNHLPGSWSDKSADISESHGLINQTLAAGRASALILNTEDELEAPELTHLSSIENKIYTIGPL
HALSKSRLGDSSSSASALSGEWKEDRACMSWLDCQPPRSVVFVSFGSTMKMKADELREFWYGLVSS
GKPFLCVLRSDVVSGGEAAELIEQMAEEEGAGGKLGMVVEWAAQEKVLSHPAVGGFLTHCGWNSTV
ESIAAGVPMMCWPILGDQPSNATWIDRVWKIGVERNNREWDRLTVEKMVRALMEGQKRVEIQRSME
KLSKLANEKVVRGINLHPTISLKKDTPTTSEHPRHEFENMRGMNYEMLVGNAIKSPTLTKK
SEQ ID NO: 34: SgUGT94-289-3 (Siraitia grosvenorii)
MTIFFSVEILVLGIAEFAAIAMDAAQQGDTTTILMLPWLGYGHLSAFLELAKSLSRRNFHIYFCST
SVNLDAIKPKLPSSESDSIQFVELHLPSSPEFPPHLHTINGLPPTLMPALHQAFSMAAQHFESILQ
TLAPHLLIYDSLQPWAPRVASSLKIPAINENTTGVFVISQGLHPIHYPHSKFPFSEFVLHNHWKAM
YSTADGASTERTRKRGEAFLYCLHASCSVILINSFRELEGKYMDYLSVLLNKKVVPVGPLVYEPNQ
DGEDEGYSSIKNWLDKKEPSSTVFVSFGSEYFPSKEEMEEIAHGLEASEVNFIWVVREPQGDNTSG
IEDALPKGFLERAGERGMVVKGWAPQAKILKHWSTGGFVSHCGWNSVMESMMFGVPIIGVPMHVDQ
PFNAGLVEEAGVGVEAKRDPDGKIQRDEVAKLIKEVVVEKTREDVRKKAREMSEILRSKGEEKFDE
MVAEISLLLKI
SEQ ID NO: 35: SgUGT74-345-2 (Siraitia grosvenorii)
MDETTVNGGRRASDVVVFAFPRHGHMSPMLQFSKRLVSKGLRVTFLITTSATESLRLNLPPSSSLD
LQVISDVPESNDIATLEGYLRSFKATVSKTLADFIDGIGNPPKFIVYDSVMPWVQEVARGRGLDAA
PFFTQSSAVNHILNHVYGGSLSIPAPENTAVSLPSMPVLQAEDLPAFPDDPEVVMNEMTSQFSNFQ
DAKWIFFNTFDQLECKKQSQVVNWMADRWPIKTVGPTIPSAYLDDGRLEDDRAFGLNLLKPEDGKN
TRQWQWLDSKDTASVLYISFGSLAILQEEQVKELAYFLKDTNLSELWVLRDSELQKLPHNFVQETS
HRGLVVNWCSQLQVLSHRAVSCFVTHCGWNSTLEALSLGVPMVAIPQWVDQTTNAKFVADVWRVGV
RVKKKDERIVTKEELEASIRQVVQGEGRNEFKHNAIKWKKLAKEAVDEGGSSDKNIEEFVKTIA
SEQ ID NO: 36: SgUGT75-281-2 (Siraitia grosvenorii)
MGDNGDGGEKKELKENVKKGKELGRQAIGEGYINPSLQLARRLISLGVNVTFATTVLAGRRMKNKT
HQTATTPGLSFATESDGEDDETLKPNGDLTHYFSELRRCGSESLTHLITSAANEGRPITFVIYSLL
LSWAADIASTYDIPSALFFAQPATVLALYFYYFHGYGDTICSKLQDPSSYIELPGLPLLTSQDMPS
FFSPSGPHAFILPPMREQAEFLGRQSQPKVLVNTEDALEADALRAIDKLKMLAIGPLIPSALLGGN
DSSDASFCGDLFQVSSEDYIEWLNSKPDSSVVYISVGSICVLSDEQEDELVHALLNSGHTFLWVKR
SKENNEGVKQETDEEKLKKLEEQGKMVSWCRQVEVLKHPALGCELTHCGWNSTIESLVSGLPVVAF
PQQIDQATNAKLIEDVWKTGVRVKANTEGIVEREEIRRCLDLVMGSRDGQKEEIERNAKKWKELAR
QAIGEGGSSDSNLKTELWEIDLEI
SEQ ID NO: 37: SgUGT720-269-4 (Siraitia grosvenorii)
MAEQAHDLLHVLLFPFPAEGHIKPFLCLAELLCNAGFHVTELNTDYNHRRLHNLHLLAARFPSLHE
ESISDGLPPDQPRDILDPKFFISICQVTKPLFRELLLSYKRISSVQTGRPPITCVITDVIFRFPID
VAEELDIPVESFCTESARFMFLYFWIPKLIEDGQLPYPNGNINQKLYGVAPEAEGLLRCKDLPGHW
AFADELKDDQLNFVDQTTASSRSSGLILNTEDDLEAPELGRLSTIFKKIYAVGPIHSLLNSHHCGL
WKEDHSCLAWLDSRAAKSVVFVSFGSLVKITSRQLMEFWHGLLNSGKSFLFVLRSDVVEGDDEKQV
VKEIYETKAEGKWLVVGWAPQEKVLAHEAVGGFLTHSGWNSILESIAAGVPMISCPKIGDQSSNCT
WISKVWKIGLEMEDRYDRVSVETMVRSIMEQEGEKMQKTIAELAKQAKYKVSKDGTSYQNLECLIQ
DIKKLNQIEGFINNPNFSDLLRV
SEQ ID NO: 38: SgUGT94-289-2 (Siraitia grosvenorii)
MDAQQGHTTTILMLPWVGYGHLLPFLELAKSLSRRKLFHIYFCSTSVSLDAIKPKLPPSISSDDSI
QLVELRLPSSPELPPHLHTTNGLPSHLMPALHQAFVMAAQHFQVILQTLAPHLLIYDILQPWAPQV
ASSLNIPAINFSTTGASMLSRTLHPTHYPSSKFPISEFVLHNHWRAMYTTADGALTEEGHKIEETL
ANCLHTSCGVVLVNSFRELETKYIDYLSVLLNKKVVPVGPLVYEPNQEGEDEGYSSIKNWLDKKEP
SSTVFVSFGTEYFPSKEEMEEIAYGLELSEVNFIWVLRFPQGDSTSTIEDALPKGFLERAGERAMV
VKGWAPQAKILKHWSTGGLVSHCGWNSMMEGMMFGVPIIAVPMHLDQPENAGLVEEAGVGVEAKRD
SDGKIQREEVAKSIKEVVIEKTREDVRKKAREMDTKHGPTYFSRSKVSSFGRLYKINRPTTLTVGR
FWSKQIKMKRE
SEQ ID NO: 39: SgUGT94-289-1 (Siraitia grosvenorii)
MDAQRGHTTTILMFPWLGYGHLSAFLELAKSLSRRNFHIYFCSTSVNLDAIKPKLPSSSSSDSIQL
VELCLPSSPDQLPPHLHTTNALPPHLMPTLHQAFSMAAQHFAAILHTLAPHLLIYDSFQPWAPQLA
SSLNIPAINENTTGASVLTRMLHATHYPSSKFPISEFVLHDYWKAMYSAAGGAVTKKDHKIGETLA
NCLHASCSVILINSFRELEEKYMDYLSVLLNKKVVPVGPLVYEPNQDGEDEGYSSIKNWLDKKEPS
STVFVSFGSEYFPSKEEMEEIAHGLEASEVHFIWVVRFPQGDNTSAIEDALPKGFLERVGERGMVV
KGWAPQAKILKHWSTGGFVSHCGWNSVMESMMFGVPIIGVPMHLDQPFNAGLAEEAGVGVEAKRDP
DGKIQRDEVAKLIKEVVVEKTREDVRKKAREMSEILRSKGEEKMDEMVAAISLELKI
SEQ ID NO: 40: MCUGT1 (Momordica charantia)
MAQPQTQARVLVFPYPTVGHIKPFLSLAELLADGGLDVVELSTEYNHRRIPNLEALASREPTLHED
TIPDGLPIDKPRVIIGGELYTSMRDGVKQRLRQVLQSYNDGSSPITCVICDVMLSGPIEAAEELGI
PVVTFCPYSARYLCAHFVMPKLIEEGQIPFTDGNLAGEIQGVPLEGGLLRRDHLPGEWFVKSLSDE
VWSHAFLNQTLAVGRTSALIINTLDELEAPFLAHLSSTEDKIYPIGPLDALSKSRLGDSSSSSTVL
TAFWKEDQACMSWLDSQPPKSVIFVSFGSTMRMTADKLVEFWHGLVNSGTRELCVLRSDIVEGGGA
ADLIKQVGETGNGIVVEWAAQEKVLAHRAVGGFLTHCGWNSTMESIAAGVPMMCWQIYGDQMINAT
WIGKVWKIGIERDDKWDRSTVEKMIKELMEGEKGAEIQRSMEKFSKLANDKVVKGGTSFENLELIV
EYLKKLKPSN
SEQ ID NO: 41: McUGT2 (Momordica charantia)
MAQPRVLLFPFPAMGHVKPFLSLAELLSDAGVEVVELSTEYNHRRIPDIGALAARFPTLHFETIPD
GLPPDQPRVLADGHLYFSMLDGTKPRFRQLIQSLNGNPRPITCIINDVMLSSPIEVAEEFGIPVIA
FCPCSARFLSVHFFMPNFIEEAQIPYTDENPMGKIEEATVFEGLLRRKDLPGLWCAKSSNISFSHR
FINQTIAAGRASALILNTFDELESPFLNHLSSIFPKIYCIGPLNALSRSRLGKSSSSSSALAGFWK
EDQAYMSWLESQPPRSVIFVSFGSTMKMEAWKLAEFWYGLVNSGSPELFVERPDCVINSGDAAEVM
EGRGRGMVVEWASQEKVLAHPAVGGFLTHCGWNSTVESIVAGVPMMCCPIVADQLSNATWIHKVWK
IGIEGDEKWDRSTVEMMIKELMESQKGTEIRTSIEMLSKLANEKVVKGGTSLNNFELLVEDIKTLR
RPYT
SEQ ID NO: 42: MCUGT3 (Momordica charantia)
MEQSDSNSDDHQHHVLLFPFPAKGHIKPFLCLAQLLCGAGLQVTELNTDHNHRRIDDRHRRLLATQ
FPMLHFKSISDGLPPDHPRDLLDGKLIASMRRVTESLFRQLLLSYNGYGNGTNNVSNSGRRPPISC
VITDVIFSFPVEVAEELGIPVESFATFSARFLFLYFWIPKLIQEGQLPFPDGKTNQELYGVPGAEG
IIRCKDLPGSWSVEAVAKNDPMNFVKQTLASSRSSGLILNTFEDLEAPFVTHLSNTEDKIYTIGPI
HSLLGTSHCGLWKEDYACLAWLDARPRKSVVEVSFGSLVKTTSRELMELWHGLVSSGKSELLVLRS
DVVEGEDEEQVVKEILESNGEGKWLVVGWAPQEEVLAHEAIGGFLTHSGWNSTMESIAAGVPMVCW
PKIGDQPSNCTWVSRVWKVGLEMEERYDRSTVARMARSMMEQEGKEMERRIAELAKRVKYRVGKDG
ESYRNLESLIRDIKITKSSN
SEQ ID NO: 43: MCUGT4 (Momordica charantia)
MDAHQQAEHTTTILMLPWVGYGHLTAYLELAKALSRRNFHIYYCSTPVNIESIKPKLTIPCSSIQF
VELHLPSSDDLPPNLHTTNGLPSHLMPTLHQAFSAAAPLFEEILQTLCPHLLIYDSLQPWAPKIAS
SLKIPALNENTSGVSVIAQALHAIHHPDSKFPLSDFILHNYWKSTYTTADGGASEKTRRAREAFLY
CLNSSGNAILINTFRELEGEYIDYLSLLLNKKVIPIGPLVYEPNQDEDQDEEYRSIKNWLDKKEPC
STVFVSFGSEYFPSNEEMEEIAPGLEESGANFIWVVRFPKLENRNGIIEEGLLERAGERGMVIKEW
APQARILRHGSIGGFVSHCGWNSVMESIICGVPVIGVPMRVDQPYNAGLVEEAGVGVEAKRDPDGK
IQRHEVSKLIKQVVVEKTRDDVRKKVAQMSEILRRKGDEKIDEMVALISLLPKG
SEQ ID NO: 44: MCUGT5 (Momordica charantia)
MDARQQAEHTTTILMLPWVGYGHLSAYLELAKALSRRNFHIYYCSTPVNIESIKPKLTIPCSSIQF
VELHLPFSDDLPPNLHTTNGLPSHLMPALHQAFSAAAPLFEAILQTLCPHLLIYDSLQPWAPQIAS
SLKIPALNFNTTGVSVIARALHTIHHPDSKFPLSEIVLHNYWKATHATADGANPEKERRDLEALLC
CLHSSCNAILINTFRELEGEYIDYLSLLLNKKVTPIGPLVYEPNQDEEQDEEYRSIKNWLDKKEPY
STIFVSFGSEYFPSNEEMEEIARGLEESGANFIWVVRFHKLENGNGITEEGLLERAGERGMVIQGW
APQARILRHGSIGGFVSHCGWNSVMESIICGVPVIGVPMGLDQPYNAGLVEEAGVGVEAKRDPDGK
IQRHEVSKLIKQVVVEKTRDDVRKKVAQMSEILRRKGDEKIDEMVALISLLLKG
SEQ ID NO: 45 (Cucumis sativus)
MGLSPTDHVLLFPFPAKGHIKPFFCLAHLLCNAGLRVTELSTEHHHQKLHNLTHLAAQIPSLHFQS
ISDGLSLDHPRNLLDGQLFKSMPQVTKPLFRQLLLSYKDGTSPITCVITDLILRFPMDVAQELDIP
VFCESTESARFLFLYFSIPKLLEDGQIPYPEGNSNQVLHGIPGAEGLLRCKDLPGYWSVEAVANYN
PMNFVNQTIATSKSHGLILNTFDELEVPFITNLSKIYKKVYTIGPIHSLLKKSVQTQYEFWKEDHS
CLAWLDSQPPRSVMFVSFGSIVKLKSSQLKEFWNGLVDSGKAFLLVLRSDALVEETGEEDEKQKEL
VIKEIMETKEEGRWVIVNWAPQEKVLEHKAIGGFLTHSGWNSTLESVAVGVPMVSWPQIGDQPSNA
TWLSKVWKIGVEMEDSYDRSTVESKVRSIMEHEDKKMENAIVELAKRVDDRVSKEGTSYQNLQRLI
EDIEGFKLN
SEQ ID NO: 46: CmaUGT1 (Cucurbita maxima)
MELSHTHHVLLFPFPAKGHIKPFFSLAQLLCNAGLRVTELNTDHHHRRIHDLNRLAAQLPTLHEDS
VSDGLPPDEPRNVEDGKLYESIRQVTSSLFRELLVSYNNGTSSGRPPITCVITDVMEREPIDIAEE
LGIPVFTESTESARFLFLIFWIPKLLEDGQLRYPEQELHGVPGAEGLIRWKDLPGEWSVEDVADWD
PMNFVNQTLATSRSSGLILNTEDELEAPFLTSLSKIYKKIYSLGPINSLLKNFQSQPQYNLWKEDH
SCMAWLDSQPRKSVVFVSFGSVVKLTSRQLMEFWNGLVNSGMPFLLVLRSDVIEAGEEVVREIMER
KAEGRWVIVSWAPQEEVLAHDAVGGFLTHSGWNSTLESLAAGVPMISWPQIGDQTSNSTWISKVWR
IGLQLEDGFDSSTIETMVRSIMDQTMEKTVAELAERAKNRASKNGTSYRNFQTLIQDITNIIETHI
SEQ ID NO: 47: (Prunus persica)
MAMKQPHVIIFPFPLQGHMKPLLCLAELLCHAGLHVTYVNTHHNHQRLANRQALSTHFPTLHFESI
SDGLPEDDPRTLNSQLLIALKTSIRPHFRELLKTISLKAESNDTLVPPPSCIMTDGLVTFAFDVAE
ELGLPILSFNVPCPRYLWTCLCLPKLIENGQLPFQDDDMNVEITGVPGMEGLLHRQDLPGFCRVKQ
ADHPSLQFAINETQTLKRASALILDTVYELDAPCISHMALMFPKIYTLGPLHALLNSQIGDMSRGL
ASHGSLWKSDLNCMTWLDSQPSKSIIYVSFGTLVHLTRAQVIEFWYGLVNSGHPFLWVMRSDITSG
DHQIPAELENGTKERGCIVDWVSQEEVLAHKSVGGFLTHSGWNSTLESIVAGLPMICWPKLGDHYI
ISSTVCRQWKIGLQLNENCDRSNIESMVQTLMGSKREEIQSSMDAISKLSRDSVAEGGSSHNNLEQ
LIEYIRNLQHQN
SEQ ID NO: 48: (Theobroma cacao)
MRQPHVLVLPFPAQGHIKPMLCLAELLCQAGLRVTFLNTHHSHRRLNNLQDLSTRFPTLHFESVSD
GLPEDHPRNLVHFMHLVHSIKNVTKPLLRDLLTSLSLKTDIPPVSCIIADGILSFAIDVAEELQIK
VIIFRTISSCCLWSYLCVPKLIQQGELQFSDSDMGQKVSSVPEMKGSLRLHDRPYSFGLKQLEDPN
FQFFVSETQAMTRASAVIENTEDSLEAPVLSQMIPLLPKVYTIGPLHALRKARLGDLSQHSSENGN
LREADHNCITWLDSQPLRSVVYVSFGSHVVLTSEELLEFWHGLVNSGKRELWVLRPDIIAGEKDHN
QIIAREPDLGTKEKGLLVDWAPQEEVLAHPSVGGFLTHCGWNSTLESMVAGVPMLCWPKLPDQLVN
SSCVSEVWKIGLDLKDMCDRSTVEKMVRALMEDRREEVMRSVDGISKLARESVSHGGSSSSNLEML
IQELET
SEQ ID NO: 49: CmaUGT2 (Cucurbita maxima)
MDAQKAVDTPPTTVLMLPWIGYGHLSAYLELAKALSRRNFHVYFCSTPVNLDSIKPNLIPPPSSIQ
FVDLHLPSSPELPPHLHTTNGLPSHLKPTLHQAFSAAAQHFEAILQTLSPHLLIYDSLQPWAPRIA
SSLNIPAINENTTAVSIIAHALHSVHYPDSKFPFSDFVLHDYWKAKYTTADGATSEKIRRGAEAFL
YCLNASCDVVLVNSFRELEGEYMDYLSVLLKKKVVSVGPLVYEPSEGEEDEEYWRIKKWLDEKEAL
STVLVSFGSEYFPSKEEMEEIAHGLEESEANFIWVVRFPKGEESCRGIEEALPKGFVERAGERAMV
VKKWAPQGKILKHGSIGGFVSHCGWNSVLESIRFGVPVIGVPMHLDQPYNAGLLEEAGIGVEAKRD
ADGKIQRDQVASLIKRVVVEKTREDIWKTVREMREVLRRRDDDMIDEMVAEISVVLKI
SEQ ID NO: 50: CmoUGT2 (Cucurbita moschata)
MDAQKAVDTPPTTVLMLPWIGYGHLSAYLELAKALSRRNFHVYFCSTPVNLDSIKPNLIPPPPSIQ
FVDLHLPSSPELPPHLHTINGLPSHLKPTLHQAFSAAAQHFEAILQTLSPHLLIYDSLQPWAPRIA
SSLNIPAINENTTAVSIIAHALHSVHYPDSKFPFSDFVLHDYWKAKYTTADGATSEKTRRGVEAFL
YCLNASCDVVLVNSFRELEGEYMDYLSVLLKKKVVSVGPLVYEPSEGEEDEEYWRIKKWLDEKEAL
STVLVSFGSEYFPPKEEMEEIAHGLEESEANFIWVVRFPKGEESSSRGIEEALPKGFVERAGERAM
VVKKWAPQGKILKHGSIGGFVSHCGWNSVLESIRFGVPVIGAPMHLDQPYNAGLLEEAGIGVEAKR
DADGKIQRDQVASLIKQVVVEKTREDIWKKVREMREVLRRRDDDDMMIDEMVAVISVVLKI
SEQ ID NO: 51: CmaUGT3 (Cucurbita maxima)
MSSNLELKISIPFGRLRDSALNCSVFHCKLHLAIAIAMDAQQAANKSPTATTIFMLPWAGYGHLSA
YLELAKALSTRNFHIYFCSTPVSLASIKPRLIPSCSSIQFVELHLPSSDEFPPHLHTTNGLPSRLV
PTFHQAFSEAAQTFEAFLQTLRPHLLIYDSLQPWAPRIASSLNIPAINFFTAGAFAVSHVLRAFHY
PDSQFPSSDFVLHSRWKIKNTTAESPTQAKLPKIGEAIGYCLNASRGVILTNSFRELEGKYIDYLS
VILKKRVFPIGPLVYQPNQDEEDEDYSRIKNWLDRKEASSTVLVSFGSEFFLSKEETEAIAHGLEQ
SEANFIWGIRFPKGAKKNAIEEALPEGFLERAGGRAMVVEEWVPQGKILKHGSIGGFVSHCGWNSA
MESIVCGVPIIGIPMQVDQPFNAGILEEAGVGVEAKRDSDGKIQRDEVAKLIKEVVVERTREDIRN
KLEKINEILRSRREEKLDELATEISLLSRN
SEQ ID NO: 52: CmoUGT3 (Cucurbita moschata)
MDAQQAANKSPTASTIFMLPWVGYGHLSAYLELAKALSTRNFHVYFCSTPVSLASIKPRLIPSCSS
IQFVELHLPSSDEFPPHLHTTNGLPAHLVPTIHQAFAAAAQTFEAFLQTLRPHLLIYDSLQPWAPR
IASSLNIPAINFFTAGAFAVSHVLRAFHYPDSQFPSSDFVLHSRWKIKNTTAESPTQVKIPKIGEA
IGYCLNASRGVILTNSFRELEGKYIDYLSVILKKRVLPIGPLVYQPNQDEEDEDYSRIKNWLDRKE
ASSTVLVSFGSEFFLSKEETEAIAHGLEQSEANFIWGIRFPKGAKKNAIEEALPEGFLERVGGRAM
VVEEWVPQGKILKHGNIGGFVSHCGWNSAMESIMCGVPVIGIPMQVDQPENAGILEEAGVGVEAKR
DSDGKIQRDEVAKLIKEVVVERTREDIRNKLEEINEILRTRREEKLDELATEISLLCKN
SEQ ID NO: 53: (Corchorus capsularis)
MDSKQKKMSVLMFPWLAYGHISPFLELAKKLSKRNFHTFFFSTPINLNSIKSKLSPKYAQSIQFVE
LHLPSLPDLPPHYHTTNGLPPHLMNTLKKAFDMSSLQFSKILKTLNPDLLVYDFIQPWAPLLALSN
KIPAVHFLCTSAAMSSFSVHAFKKPCEDEPEPNIYVHGNEMNAKENNMENCSSDDSISDQDRVLQC
FERSTKIILVKTFEELEGKFMDYLSVLLNKKIVPTGPLTQDPNEDEGDDDERTKLLLEWLNKKSKS
STVFVSFGSEYELSKEEREEIAYGLELSKVNFIWVIRFPLGENKTNLEEALPQGELQRVSERGLVV
ENWAPQAKILQHSSIGGFVSHCGWSSVMESLKFGVPIIAIPMHLDQPLNARLVVDVGVGLEVIRNH
GSLEREEIAKLIKEVVLGNGNDGEIVRRKAREMSNHIKKKGEKDMDELVEELMLICKMKPNSCHLS
SEQ ID NO: 54: (Ziziphus jujube)
MMERQRSIKVLMFPWLAHGHISPFLELAKRLTDRNFQIYFCSTPVNLTSVKPKLSQKYSSSIKLVE
LHLPSLPDLPPHYHTTNGLALNLIPTLKKAFDMSSSSESTILSTIKPDLLIYDFLQPWAPQLASCM
NIPAVNFLSAGASMVSFVLHSIKYNGDDHDDEFLTTELHLSDSMEAKFAEMTESSPDEHIDRAVTC
LERSNSLILIKSFRELEGKYLDYLSLSFAKKVVPIGPLVAQDTNPEDDSMDIINWLDKKEKSSTVE
VSFGSEYYLTNEEMEEIAYGLELSKVNFIWVVRFPLGQKMAVEEALPKGFLERVGEKGMVVEDWAP
QMKILGHSSIGGFVSHCGWSSLMESLKLGVPIIAMPMQLDQPINAKLVERSGVGLEVKRDKNGRIE
REYLAKVIREIVVEKARQDIEKKAREMSNIITEKGEEEIDNVVEELAKLCGM
SEQ ID NO: 55: (Vitis vinifera)
MDARQSDGISVLMFPWLAHGHISPFLQLAKKLSKRNFSIYFCSTPVNLDPIKGKLSESYSLSIQLV
KLHLPSLPELPPQYHTTNGLPPHLMPTLKMAFDMASPNESNILKTLHPDLLIYDFLQPWAPAAASS
LNIPAVQFLSTGATLQSFLAHRHRKPGIEFPFQEIHLPDYEIGRLNRFLEPSAGRISDRDRANQCL
ERSSRESLIKTFREIEAKYLDYVSDLTKKKMVTVGPLLQDPEDEDEATDIVEWLNKKCEASAVEVS
FGSEYFVSKEEMEEIAHGLELSNVDFIWVVRFPMGEKIRLEDALPPGELHRLGDRGMVVEGWAPQR
KILGHSSIGGFVSHCGWSSVMEGMKFGVPIIAMPMHLDQPINAKLVEAVGVGREVKRDENRKLERE
EIAKVIKEVVGEKNGENVRRKARELSETLRKKGDEEIDVVVEELKQLCSY
SEQ ID NO: 56: (Juglans regia)
MDTARKRIRVVMLPWLAHGHISPFLELSKKLAKRNFHIYFCSTPVNLSSIKPKLSGKYSRSIQLVE
LHLPSLPELPPQYHTTKGLPPHLNATLKRAFDMAGPHESNILKTLSPDLLIYDELQPWAPAIAASQ
NIPAINELSTGAAMTSFVLHAMKKPGDEFPFPEIHLDECMKTRFVDLPEDHSPSDDHNHISDKDRA
LKCFERSSGFVMMKTFEELEGKYINFLSHLMQKKIVPVGPLVQNPVRGDHEKAKTLEWLDKRKQSS
AVFVSFGTEYFLSKEEMEEIAYGLELSNVNFIWVVRFPEGEKVKLEEALPEGELQRVGEKGMVVEG
WAPQAKILMHPSIGGFVSHCGWSSVMESIDFGVPIVAIPMQLDQPVNAKVVEQAGVGVEVKRDRDG
KLEREEVATVIREVVMGNIGESVRKKEREMRDNIRKKGEEKMDGVAQELVQLYGNGIKNV
SEQ ID NO: 57: (Hevea brasiliensis)
METLQRRKISVLMFPWLAHGHLSPFLELSKKLNKRNFHVYFCSTPVNLDSIKPKLSAEYSFSIQLV
ELHLPSSPELPLHYHTTNGLPPHLMKNLKNAFDMASSSFFNILKTLKPDLLIYDFIQPWAPALASS
LNIPAVNFLCTSMAMSCFGLHLNNQEAKFPFPGIYPRDYMRMKVFGALESSSNDIKDGERAGRCMD
QSFHLILAKTFRELEGKYIDYLSVKLMKKIVPVGPLVQDPIFEDDEKIMDHHQVIKWLEKKERLST
VFVSFGTEYFLSTEEMEEIAYGLELSKAHFIWVVREPTGEKINLEESLPKRYLERVQERGKIVEGW
APQQKILRHSSIGGFVSHCGWSSIMESMKFGVPIIAMPMNLDQPVNSRIVEDAGVGIEVRRNKSGE
LEREEIAKTIRKVVVEKDGKNVSRKAREMSDTIRKKGEEEIDGVVDELLQLCDVKTNYLQ
SEQ ID NO: 58: (Manihot esculenta)
MATAQTRKISVLMFPWLAHGHLSPFLELSKKLANRNFHVYFCSTPVNLDSIKPKLSPEYHESIQFV
ELHLPSSPELPSHYHTTNGLPPHLMKTLKKAFDMASSSFFNILKTLNPDLLIYDELQPWAPALASS
LNIPAVNFLCSSMAMSCFGLNLNKNKEIKFLFPEIYPRDYMEMKLERVFESSSNQIKDGERAGRCI
DQSFHVILAKTFRELEGKYIDYVSVKCNKKIVPVGPLVEDTIHEDDEKTMDHHHHHHDEVIKWLEK
KERSTTVFVSFGSEYFLSKEEMEEIAHGLELSKVNFIWVVRFPKGEKINLEESLPEGYLERIQERG
KIVEGWAPQRKILGHSSIGGFVSHCGWSSIMESMKLGVPIIAMPMNLDQPINSRIVEAAGVGIEVS
RNQSGELEREEMAKTIRKVVVEREGVYVRRKAREMSDVLRKKGEEEIDGVVDELVQLCDMKTNYL
SEQ ID NO: 59: (Cephalotus follicularis)
MDLKRRSIRVLMLPWLAHGHISPFLELAKKLTNRNELIYFCSTPINLNSIKPKLSSKYSESIQLVE
LHLPSLPELPPHYHTTNGLPLHLMNTLKTAFDMASPSELNILKTLKPDLLICDHLQPWAPSLASSL
NIPAIIFPTNSAIMMAFSLHHAKNPGEEFPFPSININDDMVKSINFLHSASNGLTDMDRVLQCLER
SSNTMLLKTFRQLEAKYVDYSSALLKKKIVLAGPLVQVPDNEDEKIEIIKWLDSRGQSSTVFVSFG
SEYFLSKEEREDIAHGLELSKVNFIWVVRFPVGEKVKLEEALPNGFAERIGERGLVVEGWAPQAMI
LSHSSIGGFVSHCGWSSMMESMKFGVPIIAMPMHIDQPLNARLVEDVGVGLEIKRNKDGRFEREEL
ARVIKEVLVYKNGDAVRSKAREMSEHIKKNGDQEIDGVADALVKLCEMKTNSLNQD
SEQ ID NO: 60: UGT 1, 6 (Coffea Arabica)
MENHATFNVLMLPWLAHGHVSPYLELAKKLTARNENVYLCSSPATLSSVRSKLTEKESQSIHLVEL
HLPKLPELPAEYHTTNGLPPHLMPTLKDAFDMAKPNFCNVLKSLKPDLLIYDLLQPWAPEAASAEN
IPAVVFISSSATMTSFGLHFFKNPGTKYPYGNAIFYRDYESVEVENLTRRDRDTYRVINCMERSSK
IILIKGFNEIEGKYFDYFSCLTGKKVVPVGPLVQDPVLDDEDCRIMQWLNKKEKGSTVFVSFGSEY
FLSKKDMEEIAHGLEVSNVDFIWVVRFPKGENIVIEETLPKGFFERVGERGLVVNGWAPQAKILTH
PNVGGFVSHCGWNSVMESMKFGLPIIAMPMHLDQPINARLIEEVGAGVEVLRDSKGKLHRERMAET
INKVMKEASGESVRKKARELQEKLELKGDEEIDDVVKELVQLCATKNKRNGLHYY
SEQ ID NO: 61: CmoUGT1 (Cucurbita moschata)
MELSPTHHLLLFPFPAKGHIKPFFSLAQLLCNAGARVTELNTDHHHRRIHDLDRLAAQLPTLHEDS
VSDGLPPDESRNVEDGKLYESIRQVTSSLFRELLVSYNNGTSSGRPPITCVITDCMFRFPIDIAEE
LGIPVFTFSTFSARFLFLFFWIPKLLEDGQLRYPEQELHGVPGAEGLIRCKDLPGELSDEDVAHWK
PINFVNQILATSRSSGLILNTEDELEAPFLTSLSKIYKKIYSLGPINSLLKNFQSQPQYNLWKEDH
SCMAWLDSQPPKSVVFVSFGSVVKLTNRQLVEFWNGLVNSGKPFLLVLRSDVIEAGEEVVRENMER
KAEGRWMIVSWAPQEEVLAHDAVGGFLTHSGWNSTLESLAAGVPMISWTQIGDQTSNSTWVSKVWR
IGLQLEDGEDSFTIETMVRSVMDQTMEKTVAELAERAKNRASKNGTSYRNFQTLIQDITNIIETH
SEQ ID NO: 62: (Arabidopsis thaliana)
MGSISEMVFETCPSPNPIHVMLVSFQGQGHVNPLLRLGKLIASKGLLVTFVTTELWGKKMRQANKI
VDGELKPVGSGSIRFEFFDEEWAEDDDRRADESLYIAHLESVGIREVSKLVRRYEEANEPVSCLIN
NPFIPWVCHVAEEFNIPCAVLWVQSCACFSAYYHYQDGSVSFPTETEPELDVKLPCVPVLKNDEIP
SFLHPSSRFTGFRQAILGQFKNLSKSFCVLIDSEDSLEREVIDYMSSLCPVKTVGPLFKVARTVTS
DVSGDICKSTDKCLEWLDSRPKSSVVYISFGTVAYLKQEQIEEIAHGVLKSGLSFLWVIRPPPHDL
KVETHVLPQELKESSAKGKGMIVDWCPQEQVLSHPSVACFVTHCGWNSTMESLSSGVPVVCCPQWG
DQVTDAVYLIDVEKTGVRLGRGATEERVVPREEVAEKLLEATVGEKAEELRKNALKWKAEAEAAVA
PGGSSDKNFREFVEKLGAGVTKTKDNGY
SEQ ID NO: 63: (Arabidopsis thaliana)
MGSHVAQKQHVVCVPYPAQGHINPMMKVAKLLYAKGFHITFVNTVYNHNRLLRSRGPNAVDGLPSF
RFESIPDGLPETDVDVTQDIPTLCESTMKHCLAPFKELLRQINARDDVPPVSCIVSDGCMSFTLDA
AEELGVPEVLFWTTSACGFLAYLYYYRFIEKGLSPIKDESYLTKEHLDTKIDWIPSMKNLRLKDIP
SFIRTTNPDDIMLNFIIREADRAKRASAIILNTEDDLEHDVIQSMKSIVPPVYSIGPLHLLEKQES
GEYSEIGRTGSNLWREETECLDWLNTKARNSVVYVNFGSITVLSAKQLVEFAWGLAATGKEFLWVI
RPDLVAGDEAMVPPEFLTATADRRMLASWCPQEKVLSHPAIGGFLTHCGWNSTLESLCGGVPMVCW
PFFAEQQTNCKFSRDEWEVGIEIGGDVKREEVEAVVRELMDEEKGKNMREKAEEWRRLANEATEHK
HGSSKLNFEMLVNKVLLGE
SEQ ID NO: 64: CIUGT1 (Columba livia)
MIHCGKKHICAFVTCILISASILMYSWKDPQLQNNITRKIFQATSALPASQLCRGKPAQNVITALE
DNRTFIISPYFDDRESKVTRVIGIVHHEDVKQLYCWFCCQPDGKIYVARAKIDVHSDRFGFPYGAA
DIVCLEPENCNPTHVSIHQSPHANIDQLPSFKIKNRKSETFSVDFTVCISAMFGNYNNVLQFIQSV
EMYKILGVQKVVIYKNNCSQLMEKVLKFYMEEGTVEIIPWPINSHLKVSTKWHFSMDAKDIGYYGQ
ITALNDCIYRNMQRSKFVVLNDADEIILPLKHLDWKAMMSSLQEQNPGAGIFLFENHIFPKTVSTP
VFNISSWNRVPGVNILQHVHREPDRKEVENPKKMIIDPRQVVQTSVHSVLRAYGNSVNVPADVALV
YHCRVPLQEELPRESLIRDTALWRYNSSLITNVNKVLHQTVL
SEQ ID NO: 65: (Haemophilus ducreyi)
MPTLTVAMIVKNEAQDLAECLKTVDGWVDEIVIVDSGSTDDTLKIATQFNAKVYVNSDWQGFGPQR
QFAQQYVTSDYVLWLDADERVTPELKASILQAVQHNQKNTVYKVSRLSEIFGKEIRYSGWYPDYVV
RLYPTYLAKYGDELVHEKVHYPADSRVEKLQGDLLHFTYKNIHHYLVKSASYAKAWAMQRAKAGKK
ASLLDGVTHAIACFLKMYLFKAGELDGKQGFLLAVLSAHSTFVKYADLWDRTRS
SEQ ID NO: 66: (Neisseria gonorrhoeae)
MKKVSVLIVAKNEANHIRECIESCREDKEVIVIDDHSADNTAEIAEGLGAKVERRHLNGDEGAQKT
FAIEQAGGEWVELIDADERCTPELSDEISKIVRTGDYAAYFVERRNLFPNHPATHGAMRPDSVCRL
MPKKGGSVQGKVHETVQTPYPERRLKHEMYHYTYDNWEQYENKENKYTSISAEKYREQGKPVSFVR
DIILRPIWGFFKIYILNKGELDGKMGWIMSVNHSYYTMIKYVKLYYLYKSGGKF
SEQ ID NO: 67: (Rhizobium meliloti, strain 1021)
MPNETLHIDIGVCTYRRPELAETLRSLAAMNVPERARLRVIVADNDAEPSARALVEGLRPEMPEDI
LYVHCPHSNISIARNCCLDNSTGDFLAFLDDDETVSGDWLTRLLETARTTGAAAVLGPVRAHYGPT
APRWMRSGDFHSTLPVWAKGEIRTGYTCNALLRRDAASLLGRRFKLSLGKSGGEDTDFFTGMHCAG
GTIAFSPEAWVHEPVPENRASLAWLAKRRERSGQTHGRLLAEKAHGLRQAWNIALAGAKSGFCATA
AVLCFPSAARRNRFALRAVLHAGVISGLLGLKEIEQYGAREVTSA
SEQ ID NO: 68: (Rhizobium radiobacter)
MCRCGRAVRSRPVCRPGQLVVRRSPRPRSRNHSRCRPLRLSVFPRPHRRVRHHCQRDLRWEPGRWI
AVRWKAARSHRRERRCPFPRQLVWPVRERHRDAGDRRNQRERRRRDAYHEISEPKERTRKRTESFW
MNKAITVIVWLLVSLCVLAIITMPVSLQTHLVATAISLILLATIKSENGQGAWRLVALGFGTAIVL
RYVYWRTTSTLPPVNQLENFIPGELLYLAEMYSVVMLGLSLVIVSMPLPSRKTRPGSPDYRPTVDV
FVPSYNEDAELLANTLAAAKNMDYPADRFTVWLLDDGGSVQKRNAANIVEAQAAQRRHEELKKLCE
DLDVRYLTRERNVHAKAGNLNNGLAHSTGELVTVEDADHAPARDELLETVGYEDEDPRLFLVQTPH
FFVNPDPIERNLRTFETMPSENEMFYGIIQRGLDKWNGAFFCGSAAVLRREALQDSDGESGVSITE
DCETALALHSRGWNSVYVDKPLIAGLQPATFASFIGQRSRWAQGMMQILIFRQPLEKRGLSFTQRL
CYMSSTLFWLFPFPRTIFLFAPLFYLFFDLQIFVASGGEFLAYTAAYMLVNLMMQNYLYGSFRWPW
ISELYEYVQTVHLLPAVVSVIENPGKPTFKVTAKDESIAEARLSEISRPFFVIFALLLVAMAFAVW
RIYSEPYKADVTLVVGGWNLLNLIFAGCALGVVSERGDKSASRRITVKRRCEVQLGGSDTWVPASI
DNVSVHGLLINIFDSATNIEKGATAIVKVKPHSEGVPETMPLNVVRTVRGEGFVSIGCTFSPQRAV
DHRLIADLIFANSEQWSEFQRVRRKKPGLIRGTAIFLAIALFQTQRGLYYLVRARRPAPKSAKPVG
AVK
SEQ ID NO: 69: (Streptococcus agalactiae)
MIKKIEKDLISVIVPIYNVEDYLVECIESLIVQTYRNIEILLINDGSTDNCATIAKEFSERDCRVI
YIEKSNGGLSEARNYGIYHSKGKYLTFVDSDDKVSSDYIANLYNAIQKHDSSIAIGGYLEFYERHN
SIRNYEYLDKVIPVEEALLNMYDIKTYGSIFITAWGKLFHKSIENDLEFALNKYHEDEFFNYKAYL
KANSITYIDKPLYHYRIRVGSIMNNSDNVIIARKKLDVLSALDERIKLITSLRKYSVFLQKTEIFY
VNQYFRTKKFLKQQSVMFKEDNYIDAYRMYGRLLRKVKLVDKLKLIKNRFF
SEQ ID NO: 70 (Streptococcus pneumonia)
MYTFILMLLDFFQNHDFHFFMLFFVFILIRWAVIYFHAVRYKSYSCSVSDEKLESSVIIPVVDEPL
NLFESVLNRISRHKPSEIIVVINGPKNERLVKLCHDENEKLENNMTPIQCYYTPVPGKRNAIRVGL
EHVDSQSDITVLVDSDTVWTPRTLSELLKPFVCDKKIGGVTTRQKILDPERNLVTMFANLLEEIRA
EGTMKAMSVTGKVGCLPGRTIAFRNIVERVYTKFIEETFMGFHKEVSDDRSLTNLTLKKGYKTVMQ
DTSVVYTDAPTSWKKFIRQQLRWAEGSQYNNLKMTPWMIRNAPLMFFIYFTDMILPMLLISFGVNI
FLLKILNITTIVYTASWWEIILYVLLGMIFSFGGRNFKAMSRMKWYYVELIPVFIIVLSIIMCPIR
LLGLMRCSDDLGWGTRNLTE
SEQ ID NO: 71: AtUGT73C3 (Arabidopsis thaliana)
MATEKTHQFHPSLHFVLFPFMAQGHMIPMIDIARLLAQRGVTITIVTTPHNAARFKNVLNRAIESG
LAINILHVKFPYQEFGLPEGKENIDSLDSTELMVPFFKAVNLLEDPVMKLMEEMKPRPSCLISDWC
LPYTSIIAKNFNIPKIVFHGMGCFNLLCMHVLRRNLEILENVKSDEEYFLVPSFPDRVEFTKLQLP
VKANASGDWKEIMDEMVKAEYTSYGVIVNTFQELEPPYVKDYKEAMDGKVWSIGPVSLCNKAGADK
AERGSKAAIDQDECLQWLDSKEEGSVLYVCLGSICNLPLSQLKELGLGLEESRRSFIWVIRGSEKY
KELFEWMLESGFEERIKERGLLIKGWAPQVLILSHPSVGGFLTHCGWNSTLEGITSGIPLITWPLF
GDQFCNQKLVVQVLKAGVSAGVEEVMKWGEEDKIGVLVDKEGVKKAVEELMGDSDDAKERRRRVKE
LGELAHKAVEKGGSSHSNITLLLQDIMQLAQFKN
SEQ ID NO: 72: HVUGT B1 (Hordeum vulgare subsp. Vulgare)
MAQAESERMRVVMFPWLAHGHINPYLELAKRLIASASGDHHLDVVVHLVSTPANLAPLAHHQTDRL
RLVELHLPSLPDLPPALHTTKGLPARLMPVLKRACDLAAPRFGALLDELCPDILVYDFIQPWAPLE
AEARGVPAFHFATCGAAATAFFIHCLKTDRPPSAFPFESISLGGVDEDAKYTALVTVREDSTALVA
ERDRLPLSLERSSGFVAVKSSADIERKYMEYLSQLLGKEIIPTGPLLVDSGGSEEQRDGGRIMRWL
DGEEPGSVVFVSFGSEYFMSEHQMAQMARGLELSGVPFLWVVREPNAEDDARGAARSMPPGFEPEL
GLVVEGWAPQRRILSHPSCGAFLTHCGWSSVLESMAAGVPMVALPLHIDQPLNANLAVELGAAAAR
VKQERFGEFTAEEVARAVRAAVKGKEGEAARRRARELQEVVARNNGNDGQIATLLQRMARLCGKDQ
AVPN
SEQ ID NO: 73: HVUGT B3 (Hordeum vulgare subsp. Vulgare)
MAEANDGGKMHVVMLPWLAFGHVLPFTEFAKRVARQGHRVTLLSAPRNTRRLIDIPPGLAGLIRVV
HVPLPRVDGLPEHAEATIDLPSDHLRPCLRRAFDAAFERELSRLLQEEAKPDWVLVDYASYWAPTA
AARHGVPCAFLSLFGAAALSFFGTPETLLGIGRHAKTEPAHLTVVPEYVPFPTTVAYRGYEARELF
EPGMVPDDSGVSEGYRFAKTIEGCQLVGIRSSSEFEPEWLRLLGELYRKPVIPVGLFPPAPQDDVA
GHEATLRWLDGQAPSSVVYAAFGSEVKLTGAQLQRIALGLEASGLPFIWAFRAPTSTETGAASGGL
PEGFEERLAGRGVVCRGWVPQVKFLAHASVGGFLTHAGWNSIAEGLAHGVRLVLLPLVFEQGLNAR
NIVDKNIGVEVARDEQDGSFAAGDIAAALRRVMVEDEGEGEGAKVKELAKVFGDDEVNDQCVREFL
MHLSDHSKKNQGQD
SEQ ID NO: 74: CCUGT 1, 6 (Coffea canephora)
MAENHATFNVLMLPWLAHGHVSPYLELAMKLTARNENVYLCSSPATLSSVRSKLTEKFSQSIHLVE
LHLPKLPELPAEYHTTNGLPPHLMPTLKDAFDMAKPNFCNVLKSLKPDLLIYDLLQPWAPEAASAF
NIPAVVFISSSATMTSFGLHFFKNPGTKYPYGNTIFYRDYESVEVENLKKRDRDTYRVVNCMERSS
KIILIKGFKEIEGKYFDYFSCLTGKKVVPVGPLVQDPVLDDEDCRIMQWLNKKEKGSTVFVSEGSE
YFLSKEDMEEIAHGLELSNVDFIWVVRFPKGENIVIEETLPKGFFERVGERGLVVNGWAPQAKILT
HPNVGGFVSHCGWNSVMESMKFGLPIVAMPMHLDQPINARLIEEVGAGVEVLRDSKGKLHRERMAE
TINKVTKEASGEPARKKARELQEKLELKGDEEIDDVVKELVQLCATKNKRNGLHCYN
SEQ ID NO: 75: CeUGT 1, 6 (Coffea eugenioides)
MAENHATFNVLMLPWLAHGHVSPYLELAKKLTARNENVYLCSSPATLSSVRSKLTEKFSQSIHLVE
LHLPKLPELPAEYHTTNGLPPHLMPTLKDAFDMAEPNFCNVLKSLKPDLLIYDLLQPWAPEAASAF
NIPAVVFISSSATMTSFGLHFFKNPGTKYPYGNTIFYRDYESVEVENLKRRDRDTYRVVNCMERSS
KIILIKGFKEIEGKYFDYFSCLTGKKVVPVGPLVQDPVLDDEDCRIMQWLNKKEKGSTVFVSFGSE
YFLSKEDMEEIAHGLELSNVDFIWVVRFPKGENIVIEETLPKGFFERVGERGLVVNGWAPQAKILT
HPNVGGFVSHCGWNSVMESMKFGLPIIAMPMHLDQPINARLIEEVGAGVEVLRDSKGKLHRERMAE
TINKVTKEASGESVRKKARELQEKLELKGDEEIDDVVKELVQLCATKNKRNGLHYN
SEQ ID NO: 76: CeUGT 1, 6.2 (Coffea eugenioides)
MAENHATFNVLMLPWLAHGHVSPYLELAKKLTARNENVYLCSSPATLSSVRSKLTEKFSQSIHLVE
LHLPKLPELPAEYHTTNGLPPHLMPTLKDAFDMAKPNFCNVLKSLKPDLLIYDLLQPWAPEAASAF
NIPAVVFISSSATMTSFGLHFFKNPGTKYPYGNAIFYRDYESVEVENLTRRDRDTYRVINCMERSS
KIILIKGFNEIEGKYFDYFSCLTGKKVVPVGPLVQDPVLDDEDCEIMQWLNKKEKVSTVFVSEGSE
YFLSKKDMEEIAHGLELSNVDFIWVVRFPKGENIVIEETLPKGFFERVGERGLVVNGWAPQAKILT
HPNVGGFVSHCGWNSVMESMKFGLPIIAMPMHLDQPINARLIEEVGAGVEVLRDSKGKLHRERMAE
TINKVMKEASGESVRKKARELQEKMDLKGDEEIDDVVKELVQLCATKNKRNGLHYY
SEQ ID NO: 77: SgUGT94-289-3.2 (Siraitia grosvenorii)
MADAAQQGDTTTILMLPWLGYGHLSAFLELAKSLSRRNFHIYFCSTSVNLDAIKPKLPSSESDSIQ
FVELHLPSSPEFPPHLHTTNGLPPTLMPALHQAFSMAAQHFESILQTLAPHLLIYDSLQPWAPRVA
SSLKIPAINFNTTGVFVISQGLHPIHYPHSKFPFSEFVLHNHWKAMYSTADGASTERTRKRGEAFL
YCLHASCSVILINSFRELEGKYMDYLSVLLNKKVVPVGPLVYEPNQDGEDEGYSSIKNWLDKKEPS
STVFVSFGSEYFPSKEEMEEIAHGLEASEVNFIWVVRFPQGDNTSGIEDALPKGFLERAGERGMVV
KGWAPQAKILKHWSTGGFVSHCGWNSVMESMMFGVPIIGVPMHVDQPFNAGLVEEAGVGVEAKRDP
DGKIQRDEVAKLIKEVVVEKTREDVRKKAREMSEILRSKGEEKFDEMVAEISLLLKI
SEQ ID NO: 78: OsJUGT 1, 6 (Oryza sativa) (OsJUGT 1, 6)
MAQAERERLRVLMFPWLAHGHINPYLELATRLTTTSSSQIDVVVHLVSTPVNLAAVAHRRTDRISL
VELHLPELPGLPPALHTTKHLPPRLMPALKRACDLAAPAFGALLDELSPDVVLYDFIQPWAPLEAA
ARGVPAVHFSTCSAAATAFFLHELDGGGGGGGRGAFPFEAISLGGAEEDARYTMLTCRDDGTALLP
KGERLPLSFARSSEFVAVKTCVEIESKYMDYLSKLVGKEIIPCGPLLVDSGDVSAGSEADGVMRWL
DGQEPGSVVLVSFGSEYFMTEKQLAEMARGLELSGAAFVWVVRFPQQSPDGDEDDHGAAAARAMPP
GFAPARGLVVEGWAPQRRVLSHRSCGAFLTHCGWSSVMESMSAGVPMVALPLHIDQPVGANLAAEL
GVAARVRQERFGEFEAEEVARAVRAVMRGGEALRRRATELREVVARRDAECDEQIGALLHRMARLC
GKGTGRAAQLGH
SEQ ID NO: 79: PgUGT94 B1 (Panax ginseng)
MADNQNGRISIALLPFLAHGHISPFFELAKQLAKRNCNVELCSTPINLSSIKDKDSSASIKLVELH
LPSSPDLPPHYHTTNGLPSHLMLPLRNAFETAGPTESEILKTLNPDLLIYDENPSWAPEIASSHNI
PAVYFLTTAAASSSIGLHAFKNPGEKYPFPDFYDNSNITPEPPSADNMKLLHDFIACFERSCDIIL
IKSFRELEGKYIDLLSTLSDKTLVPVGPLVQDPMGHNEDPKTEQIINWLDKRAESTVVFVCFGSEY
FLSNEELEEVAIGLEISTVNFIWAVRLIEGEKKGILPEGFVQRVGDRGLVVEGWAPQARILGHSST
GGFVSHCGWSSIAESMKFGVPVIAMARHLDQPLNGKLAAEVGVGMEVVRDENGKYKREGIAEVIRK
VVVEKSGEVIRRKARELSEKMKEKGEQEIDRALEELVQICKKKKDEQ
SEQ ID NO: 80: SrUGT73E1, with optional His tag (Stevia rebaudiana)
MAHHHHHHVGTGSNDDDDKSPDPNWASTSELVFIPSPGAGHLPPTVELAKLLLHRDQRLSVTIIVM
NLWLGPKHNTEARPCVPSLRFVDIPCDESTMALISPNTFISAFVEHHKPRVRDIVRGIIESDSVRL
AGFVLDMFCMPMSDVANEFGVPSYNYFTSGAATLGLMEHLQWKRDHEGYDATELKNSDTELSVPSY
VNPVPAKVLPEVVLDKEGGSKMELDLAERIRESKGIIVNSCQAIERHALEYLSSNNNGIPPVFPVG
PILNLENKKDDAKTDEIMRWLNEQPESSVVELCFGSMGSENEKQVKEIAVAIERSGHRELWSLRRP
TPKEKIEFPKEYENLEEVLPEGELKRTSSIGKVIGWAPQMAVLSHPSVGGFVSHCGWNSTLESMWC
GVPMAAWPLYAEQTLNAFLLVVELGLAAEIRMDYRTDTKAGYDGGMEVTVEEIEDGIRKLMSDGEI
RNKVKDVKEKSRAAVVEGGSSYASIGKFIEHVSNVTI
SEQ ID NO: 81: (Camelina sativa)
MASEKTLQVHPPLHFVLFPFMAQGHMIPMVDIARLLAQRGATVTIVTTRYNAGRFENVLSRAVESG
LPINIVHVKFPYEEVGLPKGKENIDSLDSMELMVPFFKAVNMLQDPVVKLMEEMESRPSCIISDLL
LPYTSKIAKKENIPKIVFHGISCFCLLCVHVLRRNLEILTNLKSDKEYFLVPSFPDRVEFTKPQVT
VETNASGDWKEFLDEMVEAEDTSYGVIINTFEELEPAYVKDYKDARAGNVWSIGPVSLCNKAGVDK
AERGNKATIDQDECLKWLDSKEEGSVLYVCLGSICNLPLVQLKELGLGLEESQRPFIWVIRGWEKY
NELSEWMVESGFEERIRERGLLIRGWAPQVLILSHPSVGGFLTHCGWNSTVEGITSGVPLITWPLE
GDQFCNQTLVVQVLKAGVSVGVEEVMKWGEEEKIGVLVDKEGVKKAVEDLMGESDDAKERTKRVKE
LGGLAHKAVEEGGSSHSNITLFLQDIRQVQSV
SEQ ID NO: 82: UGT73F24 (Glycyrrhiza uralensis (UGT73F24)
MADVAEEQPLKIYFIPYLAAGHMIPLCDIATLFASRGHHVTIITTPSNAQTLRESHHERVQTIQFP
SQEVGLPAGVQNLTAVTNLDDSYKIYHATMLLRKHIEDFVERDPPDCIVADFLFPWVDDVATKLHI
PRLVENGFTLFTICAMESHKAHPLPVDAASGSFVIPDFPHHVTINSTPPKRTKEFVDPLLTEAFKS
HGFLINSFVELDGEECVEHYERITGGHKAWHLGPAFLVHRTAQDRGEKSVVSTQECLSWLDSKRDN
SVLYICFGTICYFPDKQLYEIASAIEASGHEFIWVVPEKRGNADESEEEKEKWLPKGFEERNNGKK
GMIIRGWAPQVAILGHPAVGGFLTHCGWNSTVEAVSAGVPMITWPVHSDQYFNEKLITQVRGIGVE
VGAEEWIVTAFRETEKLVGRDRIERAVRRVMDGGDEAVQIRRRARELGEMARQAVQEGGSSHTNLT
ALINDLKRWRDSKQLN
SEQ ID NO: 83: UGT73C33 (Glycyrrhiza uralensis)
MAVFQANQPHFVLFPLMAQGHIIPMIDIARLLAQRGAIVTIFTTPKNASRFTSVLSRAVSSGLQIR
LVHLHFPSKEAGLPEGCENLDMVASHDMICNIFQAIRMLQKQAEELFETLTPKPSCIISDFCIPWT
TQVAEKHHIPRISFHGESCFCLHCMLKIHTSKVLEGITSESEYFTVPGIPDQIQVTKQQVPGPMID
EMKEFGEQMRDAEIRSYGVIINTFEELEKAYVNDYKKERNGKVWCIGPVSLCNKDGLDKAQRGNKA
SISEHHCLEWLDLQQPNSVIYVCLGSLCNLTPPQLMELALGLEATKRPFIWVIREGNKFEELEKWI
SEEGFEERIKGRGLIIRGWAPQVLILSHPSIGGFLTHCGWNSTLEGVTAGVPMVTWPLFADQELNE
KLVTQVLRIGVSLGVDVPLKWGEEEKVGVQVKKEGIEKAICMVMDEGEESKERRERAKELSEMAKR
AVEKDGSSHLNMTMLIQDIMQQSSSKVET
SEQ ID NO: 84: UGT85C1 (Stevia rebaudiana)
MDQMAKIDEKKPHVVFIPFPAQSHIKCMLKLARILHQKGLYITFINTDTNHERLVASGGTQWLENA
PGFWFKTVPDGFGSAKDDGVKPTDALRELMDYLKTNFFDLELDLVLKLEVPATCIICDGCMTFANT
IRAAEKLNIPVILFWTMAACGEMAFYQAKVLKEKEIVPVKDETYLINGYLDMEIDWIPGMKRIRLR
DLPEFILATKQNYFAFEFLFETAQLADKVSHMIIHTFEELEASLVSEIKSIFPNVYTIGPLQLLLN
KITQKETNNDSYSLWKEEPECVEWLNSKEPNSVVYVNFGSLAVMSLQDLVEFGWGLVNSNHYELWI
IRANLIDGKPAVMPQELKEAMNEKGFVGSWCSQEEVLNHPAVGGFLTHCGWGSIIESLSAGVPMLG
WPSIGDQRANCRQMCKEWEVGMEIGKNVKRDEVEKLVRMLMEGLEGERMRKKALEWKKSATLATCC
NGSSSLDVEKLANEIKKLSRN
SEQ ID NO: 99: At75D1 (Arabidopsis thaliana)
MANNNSNSPTGPHELFVTFPAQGHINPSLELAKRLAGTISGARVTFAASISAYNRRMESTENVPET
LIFATYSDGHDDGFKSSAYSDKSRQDATGNEMSEMRRRGKETLTELIEDNRKQNRPFTCVVYTILL
TWVAELAREFHLPSALLWVQPVTVESIFYHYENGYEDAISEMANTPSSSIKLPSLPLLTVRDIPSF
IVSSNVYAFLLPAFREQIDSLKEEINPKILINTFQELEPEAMSSVPDNEKIVPVGPLLTLRTDESS
RGEYIEWLDTKADSSVLYVSFGTLAVLSKKQLVELCKALIQSRRPFLWVITDKSYRNKEDEQEKEE
DCISSFREELDEIGMVVSWCDQFRVLNHRSIGCFVTHCGWNSTLESLVSGVPVVAFPQWNDQMMNA
KLLEDCWKTGVRVMEKKEEEGVVVVDSEEIRRCIEEVMEDKAEEFRGNATRWKDLAAEAVREGGSS
FNHLKAFVDEHM
LINKERS
SEQ ID NO: 85: GGSGGS (L6)
SEQ ID NO: 86: GGSGGSG (L7)
SEQ ID NO: 87: GGSGGSGG (L8)
SEQ ID NO: 88: GGSGGSGGS (L9)
SEQ ID NO: 89: GGSGGSGGSG (L10)
SEQ ID NO: 90: GGSGGSGGSGG (L11)
SEQ ID NO: 91: GGSGGSGGSGGS (L12)
Complementing Enzymes
SEQ ID NO: 92: E. coli pgm
MAIHNRAGQPAQQSDLINVAQLTAQYYVLKPEAGNAEHAVKFGTSGHRGSAARHSENEPHILAIAQ
AIAEERAKNGITGPCYVGKDTHALSEPAFISVLEVLAANGVDVIVQENNGETPTPAVSNAILVHNK
KGGPLADGIVITPSHNPPEDGGIKYNPPNGGPADTNVTKVVEDRANALLADGLKGVKRISLDEAMA
SGHVKEQDLVQPFVEGLADIVDMAAIQKAGLTLGVDPLGGSGIEYWKRIGEYYNLNLTIVNDQVDQ
TFRFMHLDKDGAIRMDCSSECAMAGLLALRDKEDLAFANDPDYDRHGIVTPAGLMNPNHYLAVAIN
YLFQHRPQWGKDVAVGKTLVSSAMIDRVVNDLGRKLVEVPVGFKWFVDGLEDGSFGFGGEESAGAS
FLRFDGTPWSTDKDGIIMCLLAAEITAVTGKNPQEHYNELAKRFGAPSYNRLQAAATSAQKAALSK
LSPEMVSASTLAGDPITARLTAAPGNGASIGGLKVMTDNGWFAARPSGTEDAYKIYCESFLGEEHR
KQIEKEAVEIVSEVLKNA
SEQ ID NO: 93: E. coli galU
MAAINTKVKKAVIPVAGLGTRMLPATKAIPKEMLPLVDKPLIQYVVNECIAAGITEIVLVTHSSKN
SIENHEDTSFELEAMLEKRVKRQLLDEVQSICPPHVTIMQVRQGLAKGLGHAVLCAHPVVGDEPVA
VILPDVILDEYESDLSQDNLAEMIRRFDETGHSQIMVEPVADVTAYGVVDCKGVELAPGESVPMVG
VVEKPKADVAPSNLAIVGRYVLSADIWPLLAKTPPGAGDEIQLTDAIDMLIEKETVEAYHMKGKSH
DCGNKLGYMQAFVEYGIRHNTLGTEFKAWLEEEMGIKK
SEQ ID NO: 94: E. coli ycjU
MKLQGVIFDLDGVITDTAHLHFQAWQQIAAEIGISIDAQFNESLKGISRDESLRRILQHGGKEGDE
NSQERAQLAYRKNLLYVHSLRELTVNAVLPGIRSLLADLRAQQISVGLASVSLNAPTILAALELRE
FFTFCADASQLKNSKPDPEIFLAACAGLGVPPQACIGIEDAQAGIDAINASGMRSVGIGAGLTGAQ
LLLPSTESLTWPRLSAFWQNV
SEQ ID NO: 95: Bifidobacterium bifidum ugpA
MAFAEDLKRTEKMTVDDVFEQSAQKMREQGMSEIAISQFRHAYHVWASEKESAWIREDTVEPLHGV
RSFHDVYKTIDHDKAVHAFAKTAFLKLNGGLGTSMGLQCAKSLLPVRRHKARQMRELDIILGQVLT
ARTRLNVPLPVTEMNSFRTSDDTMKALRHQRKFKQTDIPLEIIQHQEPKIDAATGAPASWPANPDL
EWCPPGHGDLFSTLRESGLLDTLLEHGFEYLFISNSDNLGARPSRTLAQYFEDTGAPEMVEVANRT
YADRKGGHIVRDTATGRLILREMSQVHPDDKDAAQDIAKHPYENTNNIWVRIDVLRDMLAEHDGVL
PLPVIINNKTVDPIDPQSPAVVQLETAMGAAIGLFEGAICVQVDRMRFLPVKTTNDLFIMRSDREH
LTDSYEMEDGNYIFPNVDLDPRYYKNIEDENERFPYNVPSLAAANSVSIKGDWTFGRDVIMFADAR
LEDRNEPSYVPNGEYVGPMGIEPGDWV
SEQ ID NO: 96: E. coli adk
MRIILLGAPGAGKGTQAQFIMEKYGIPQISTGDMLRAAVKSGSELGKQAKDIMDAGKLVTDELVIA
LVKERIAQEDCRNGFLLDGFPRTIPQADAMKEAGINVDYVLEFDVPDELIVDRIVGRRVHAPSGRV
YHVKFNPPKVEGKDDVTGEELTTRKDDQEETVRKRLVEYHQMTAPLIGYYSKEAEAGNTKYAKVDG
TKPVAEVRADLEKILG
SEQ ID NO: 97: E. coli ndk
MAIERTFSIIKPNAVAKNVIGNIFARFEAAGFKIVGTKMLHLTVEQARGFYAEHDGKPFEDGLVEF
MTSGPIVVSVLEGENAVQRHRDLLGATNPANALAGTLRADYADSLTENGTHGSDSVESAAREIAYF
FGEGEVCPRTR
SEQ ID NO: 98: E. coli cmk
MTAIAPVITIDGPSGAGKGTLCKAMAEALQWHLLDSGAIYRVLALAALHHHVDVASEDALVPLASH
LDVRFVSTNGNLEVILEGEDVSGEIRTQEVANAASQVAAFPRVREALLRRQRAFRELPGLIADGRD
MGTVVFPDAPVKIFLDASSEERAHRRMLQLQEKGFSVNFERLLAEIKERDDRDRNRAVAPLVPAAD
ALVLDSTTLSIEQVIEKALQYARQKLALA

What is claimed is:

1. A method for preparing a glycosylated product, comprising:
providing a bacterial cell expressing one or more recombinant UDP-dependent glycosyltransferase (UGT) enzymes, the bacterial cell having one or more of:
a recombinant sucrose synthase enzyme expression, and one or more genetic modifications that increase availability of UDP-sugar;
culturing the bacterial cell in the presence of a substrate for glycosylation; and
recovering the glycosylated product.

2. The method of claim 1, wherein the bacterial cell is Escherichia spp., Bacillus spp., Rhodobacter spp., Zymomonas spp., or Pseudomonas spp.

3-4. (canceled)

5. The method of claim 1, wherein the bacterial cell expresses a recombinant sucrose synthase enzyme comprising an amino acid sequence that has at least about 70% sequence identity with one of SEQ ID NOS: 1 to 12.

6-24. (canceled)

25. The method of claim 1, wherein the substrates for glycosylation are provided as a plant extract or fraction thereof, or are produced synthetically or by a biosynthesis process.

26-33. (canceled)

34. The method of claim 1, wherein the substrate is provided as a stevia leaf extract or fraction thereof.

35-65. (canceled)

66. A bacterial cell for preparing a glycosylated product, the bacterial cell expressing one or more recombinant UDP-dependent glycosyltransferase (UGT) enzymes and having one or more of: expression of a recombinant sucrose synthase enzyme, and one or more genetic modifications that increase availability of UDP-sugar.

67-108. (canceled)

109. A method for glycosylation of a substrate, comprising, culturing the cell of claim 66 in the presence of a substrate for glycosylation, and recovering the glycosylated product.

110. (canceled)

111. A UDP-dependent glycosyltransferase (UGT) enzyme comprising an amino acid sequence that has at least about 70% sequence identity to SEQ ID NO: 13, and having one or more modifications selected from:
one or more amino acid substitutions selected from: V397S, V397C, G5N, S20E, S23D, R45Y, H59P, G94S, K97E, M150L, I185F, A206P, G210E, Q237R, M250K, A251E, C252L, G259E, Q263Y, I287M, C288F, V336L, F338L, D351E, F186I, F186M, F186T, L418F, A451T, A451L, T453K, T453R, V456S, V456W, V456T, V456M;
substitution of residues 270 to 281 of SEQ ID NO: 13 with from five to fifteen amino acids comprised predominately of glycine and serine amino acids;
insertion of one or two amino acids at position 3 with respect to SEQ ID NO: 13, and/or addition of an amino acid to the C-terminus of SEQ ID NO: 13.

112-116. (canceled)

117. A UDP-dependent glycosyltransferase (UGT) enzyme comprising an amino acid sequence that has at least about 70% sequence identity to SEQ ID NO: 14, and having one or more modifications selected from:
one or more amino acid substitutions selected from: V395A, Q263Y, D269R, K97E, Q262E, H59P, G259E, M150L, Y267H, T3R, V95Q, A238E, S308Q, Q237R, R45Y, E254D, L203L, S151R, S123D, D351E, T453M, G94T, T186M, V336L, L58S, F338L, F51W, C252L, M250D, A251E, C252V, A79P, W401F, S323A, A251E, A130D, S42E, H400Y, S266R, S23D, P56A, A206P, M250K, A143W, V456T, G94S, I427F, T186L, T453F, C252R, V38F, R45F, T37S, Q244K, L11I, I287M, V31P, T43D, and P39T;
deletion of residues 270 to 281 of SEQ ID NO: 2, with a linker of from five to fifteen amino acids and comprised predominately of glycine and serine amino acids;
insertion of one or two amino acids at position 3 with respect to SEQ ID NO: 14, and/or addition of an amino acid to the C-terminus of SEQ ID NO: 14.

118-122. (canceled)

123. A UDP-dependent glycosyltransferase (UGT) enzyme comprising an amino acid sequence that has at least about 70% sequence identity to SEQ ID NO: 15, and having one or more amino acid substitutions at positions selected from 125, 152, 153, and 442 with respect to SEQ ID NO: 15.

124-125. (canceled)

126. A method for glycosylating a mogrol or mogrol glycoside substrate, the method comprising contacting the substrate with a UDP-dependent glycosyltransferase (UGT) enzyme in the presence of UDP-sugar, the UGT enzyme comprising an amino acid sequence that has at least about 80% sequence identity to an amino acid sequence selected from: SEQ ID NO: 84, SEQ ID NO: 80, SEQ ID NO: 46, SEQ ID NO: 83, SEQ ID NO: 82, SEQ ID NO: 73, SEQ ID NO: 72, SEQ ID NO: 78, SEQ ID NO: 54, SEQ ID NO: 74, SEQ ID NO: 75, SEQ ID NO: 76, SEQ ID NO: 13, SEQ ID NO: 14, SEQ ID NO: 15, SEQ ID NO: 16, SEQ ID NO: 29, and SEQ ID NO: 79.

127-144. (canceled)

145. A method for producing a glycoside product, comprising:
converting a substrate for glycosylation to a target glycoside product by enzymatic transfer of one or more sugar moieties in a cell-free reaction or in a microbial culture, and
recovering the glycoside products from the reaction or culture, the recovering comprising one or more of: lowering the pH of the reaction or culture to below about pH 5 or raising the pH of the reaction or culture to above about pH 9, raising the temperature to at least about 50° C., and adding one or more glycoside solubilizers; followed by enzyme or biomass removal.

146. The method of claim 145, wherein the substrates for glycosylation are provided as a plant extract or fraction thereof.

147-156. (canceled)

157. The method of claim 145, wherein the glycosylated product comprises RebM.

158-172. (canceled)

173. The method of claim 145, wherein the enzymatic transfer is by microbial culture of a bacterial strain.

174-184. (canceled)

185. The method of claim 145, wherein the pH is adjusted to a pH within the range of about 2 and about 4, and optionally about 3.5.

186. The method of claim 145, wherein the pH is adjusted to a pH within the range of about 9 to about 12.

187. The method of claim 145, wherein the temperature is adjusted to a temperature between about 50° C. and about 90° C., and optionally about 70° C. or about 80° C.

188-195. (canceled)

196. The method of claim 187, wherein temperature adjustment takes place by transfer of the reaction media or culture to pre-heated harvest tanks.

197. The method of claim 145, wherein biomass and/or enzymes are removed by centrifugation, thereby preparing a clarified broth.

198-200. (canceled)

201. The method of claim 197, wherein the clarified broth is transferred directly or indirectly to one or more crystallization vessels.

202. The method of claim 201, wherein, prior to crystallization, glycoside products are purified from the clarified broth using one or more processes selected from filtration, ion exchange, activated charcoal, bentonite, affinity chromatography, and digestion.

203. The method of claim 202, wherein the affinity chromatography employs one or more of a styrene-divinylbenzene adsorbent resin, a strongly acidic cation exchange resin, a weakly acidic cation exchange resin, a strongly basic anion exchange resin, a weakly basic anion exchange resin, and a hydrophobic interaction resin.

204. (canceled)

205. The method of claim 203, wherein, prior to crystallization, glycoside products are purified by tangential flow filtration (TFF), optionally having a membrane pore size of about 5 kD.

206. (canceled)

207. The method of claim 202, wherein at least two crystallization steps are employed.

208-229. (canceled)


SEQUENCE LISTING (99 sequences)

SEQ ID NO: 1 - protein, 805 aa, Solanum tuberosum
MAERVLTRVHSLRERVDATLAAHRNEILLFLSRIESHGKGILKPHELLAEFDAIRQDDKNKLNEHAFEELLKSTQEAIVLPPWVALAIRLRPGVWEYIRVNVNALVVEELSVPEYLQFKEELVDGASNGNFVLELDFEPFTASFPKPTLTKSIGNGVEFLNRHLSAKMFHDKESMTPLLEFLRAHHYKGKTMMLNDRIQNSNTLQNVLRKAEEYLIMLPPETPYFEFEHKFQEIGLEKGWGDTAERVLEMVCMLLDLLEAPDSCTLEKFLGRIPMVFNVVILSPHGYFAQENVLGYPDTGGQVVYILDQVPALEREMLKRIKEQGLDIIPRILIVTRLLPDAVGTTCGQRIEKVYGAEHSHILRVPFRTEKGIVRKWISRFEVWPYMETFIEDVAKEISAELQAKPDLIIGNYSEGNLAASLLAHKLGVTQCTIAHALEKTKYPDSDIYWKKFDEKYHFSSQFTADLIAMNHTDFIITSTFQEIAGSKDTVGQYESHMAFTMPGLYRVVHGINVFDPKFNIVSPGADINLYFSYSETEKRLTAFHPEIDELLYSDVENDEHLCVLKDRTKPILFTMARLDRVKNLTGLVEWYAKNPRLRGLVNLVVVGGDRRKESKDLEEQAEMKKMYELIETHNLNGQFRWISSQMNRVRNGELYRYIADTKGAFVQPAFYEAFGLTVVEAMTCGLPTFATNHGGPAEIIVHGKSGFHIDPYHGEQAADLLADFFEKCKKDPSHWETISMGGLKRIEEKYTWQIYSESLLTLAAVYGFWKHVSKLDRLEIRRYLEMFYALKYRKMAEAVPLAAE

SEQ ID NO: 2 - protein, 805 aa, Solanum tuberosum
MAERVLTRVHSLRERLDATLAAHRNEILLFLSRIESHGKGILKPHQLLAEFESIHKEDKDKLNDHAFEEVLKSTQEAIVLPPWVALAIRLRPGVWEYVRVNVNALIVEELTVPEFLQFKEELVNGTSNDNFVLELDFEPFTASFPKPTLTKSIGNGVEFLNRHLSAKMFHDKESMTPLLEFLRVHHYKGKTMMLNDRIQNLYTLQKVLRKAEEYLTTLSPETSYSAFEHKFQEIGLERGWGDTAERVLEMICMLLDLLEAPDSCTLEKFLGRIPMVFNVVILSPHGYFAQENVLGYPDTGGQVVYILDQVPALEREMLKRIKEQGLDIKPRILIVTRLLPDAVGTTCGQRLEKVFGTEHSHILRVPFRTEKGIVRKWISRFEVWPYMETFIEDVGKEITAELQAKPDLIIGNYSEGNLAASLLAHKLGVTQCTIAHALEKTKYPDSDIYLNKFDEKYHFSAQFTADLIAMNHTDFIITSTFQEIAGSKDTVGQYESHMAFTMPGLYRVVHGIDVFDPKFNIVSPGADVNLYFPYSEKEKRLTTFHPEIEDLLFSDVENEEHLCVLKDRNKPIIFTMARLDRVKNLTGLVEWYAKNPRLRELVNLVVVGGDRRKESKDLEEQAEMKKMYELIKTHNLNGQFRWISSQMNRVRNGELYRYIADTRGAFVQPAFYEAFGLTVVEAMSCGLPTFATNQGGPAEIIVHGKSGFQIDPYHGEQAADLLADFFEKCKVDPSHWEAISEGGLKRIQEKYTWQIYSDRLLTLAAVYGFWKHVSKLDRLEIRRYLEMFYALKFRKLAQLVPLAVE

SEQ ID NO: 3 - protein, 805 aa, Solanum tuberosum
MAERVLTRVHELRERLDATLAAHRNEILLFLSRIESHGKGILKPHQLLAEFESIHKEDKDKLNDHAFEEVLKSTQEAIVLPPWVALAIRLRPGVWEYVRVNVNALIVEELTVPEFLQFKEELVNGTSNDNFVLELDFEPFTASFPKPTLTKSIGNGVEFLNRHLSAKMFHDKESMTPLLEFLRVHHYKGKTMMLNDRIQNLYTLQKVLRKAEEYLTTLSPETSYSAFEHKFQEIGLERGWGDTAERVLEMICMLLDLLEAPDSCTLEKFLGRIPMVFNVVILSPHGYFAQENVLGYPDTGGQVVYILDQVPALEREMLKRIKEQGLDIKPRILIVTRLLPDAVGTTCGQRLEKVFGTEHSHILRVPFRTEKGIVRKWISRFEVWPYMETFIEDVGKEITAELQAKPDLIIGNYSEGNLAASLLAHKLGVTQCTIAHALEKTKYPDSDIYLNKFDEKYHFSAQFTADLIAMNHTDFIITSTFQEIAGSKDTVGQYESHMAFTMPGLYRVVHGIDVFDPKFNIVSPGADVNLYFPYSEKEKRLTTFHPEIEDLLFSDVENEEHLCVLKDRNKPIIFTMARLDRVKNLTGLVEWYAKNPRLRELVNLVVVGGDRRKESKDLEEQAEMKKMYELIKTHNLNGQFRWISSQMNRVRNGELYRYIADTRGAFVQPAFYEAFGLTVVEAMSCGLPTFATNQGGPAEIIVHGKSGFQIDPYHGEQAADLLADFFEKCKVDPSHWEAISEGGLKRIQEKYTWQIYSDRLLTLAAVYGFWKHVSKLDRLEIRRYLEMFYALKFRKLAQLVPLAVE

SEQ ID NO: 4 - protein, 794 aa, Acidithiobacillus caldus
MAIEALRQQLLDDPRSWYAFLRHLVASQRDSWLYTDLQRACADFREQLPEGYAEGIGPLEDFVAHTQEVIFRDPWMVFAWRPRPGRWIYVRIHREQLALEELSTDAYLQAKEGIVGLGAEGEAVLTVDFRDFRPVSRRLRDESTIGDGLTHLNRRLAGRIFSDLAAGRSQILEFLSLHRLDGQNLMLSNGNTDFDSLRQTVQYLGTLPRETPWAEIREDMRRRGFAPGWGNTAGRVRETMRLLMDLLDSPSPAALESFLDRIPMISRILIVSIHGWFAQDKVLGRPDTGGQVVYILDQARALEREMRNRLRQQGVDVEPRILIATRLIPESDGTTCDQRLEPVVGAENVQILRVPFRYPDGRIHPHWISRFKIWPWLERYAQDLEREVLAELGSRPDLIIGNYSDGNLVATLLSERLGVTQCNIAHALEKSKYLYSDLHWRDHEQDHHFACQFTADLIAMNAADIIVTSTYQEIAGNDREIGQYEGHQDYTLPGLYRVENGIDVFDSKFNIVSPGADPRFYFSYARTEERPSFLEPEIESLLFGREPGADRRGVLEDRQKPLLLSMARMDRIKNLSGLAELYGRSSRLRGLANLVIIGGHVDVGNSRDAEEREEIRRMHEIMDHYQLDGQLRWVGALLDKTVAGELYRVVADGRGVFVQPALFEAFGLTVIEAMSSGLPVFATRFGGPLEIIEDGVSGFHIDPNDHEATAERLADFLEAARERPKYWLEISDAALARVAERYTWERYAERLMTIARIFGFWRFVLDRESQVMERYLQMFRHLQWRPLAHAVPME

SEQ ID NO: 5 - protein, 794 aa, Acidithiobacillus caldus
MAIEALRQQLLDDPRSWYAFLRHLVASQRDSWLYTDLQRACADFREQLPEGYAEGIGPLEDFVAHTQEVIFRDPWMVFAWRPRPGRWIYVRIHREQLALEELSTDAYLQAKEGIVGLGAEGEAVLTVDFRDFRPVSRRLRDESTIGDGLTHLNRRLAGRIFSDLAAGRSQILEFLSLHRLDGQNLMLSNGNTDFDSLRQTVQYLGTLPRETPWAEIREDMRRRGFAPGWGNTAGRVRETMRLLMDLLDSPSPAALESFLDRIPMISRILIVSIHGWFAQDKVLGRPDTGGQVVYILDQARALEREMRNRLRQQGVDVEPRILIATRLIPESDGTTCDQRLEPVVGAENVQILRVPFRYPDGRIHPHWISRFKIWPWLERYAQDLEREVLAELGSRPDLIIGNYSDGNLVATLLSERLGVTQCNIAHALEKSKYLYSDLHWRDHEQDHHFACQFTADLIAMNAADIIVTSTYQEIAGNDREIGQYEGHQDYTLPGLYRVENGIDVFDSKFNIVSPGADPRFYFSYARTEERPSFLEPEIESLLFGREPGADRRGVLEDRQKPLLLSMARMDRIKNLSGLAELYGRSSRLRGLANLVIIGGHVDVGNSRDAEEREEIRRMHEIMDHYQLDGQLRWVGALMDKVVAGELYRVVADGRGVFVQPALFEAFGLTVIEAMSSGLPVFATRFGGPLEIIEDGVSGFHIDPNDHEATAERLADFLEAARERPKYWLEISDAALARVAERYTWERYAERLMTIARIFGFWRFVLDRESQVMERYLQMFRHLQWRPLAHAVPME

SEQ ID NO: 6 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 7 - protein, 809 aa, Arabidopsis thaliana
MANPKLTRVLSTRDRVQDTLSAHRNELVALLSRYVDQGKGILQPHNLIDELESVIGDDETKKSLSDGPFGEILKSAMEAIVVPPFVALAVRPRPGVWEYVRVNVFELSVEQLTVSEYLRFKEELVDGPNSDPFCLELDFEPFNANVPRPSRSSSIGNGVQFLNRHLSSVMFRNKDCLEPLLDFLRVHKYKGHPLMLNDRIQSISRLQIQLSKAEDHISKLSQETPFSEFEYALQGMGFEKGWGDTAGRVLEMMHLLSDILQAPDPSSLEKFLGMVPMVFNVVILSPHGYFGQANVLGLPDTGGQVVYILDQVRALETEMLLRIKRQGLDISPSILIVTRLIPDAKGTTCNQRLERVSGTEHTHILRVPFRSEKGILRKWISRFDVWPYLENYAQDAASEIVGELQGVPDFIIGNYSDGNLVASLMAHRMGVTQCTIAHALEKTKYPDSDIYWKDFDNKYHFSCQFTADLIAMNNADFIITSTYQEIAGTKNTVGQYESHGAFTLPGLYRVVHGIDVFDPKFNIVSPGADMTIYFPYSEETRRLTALHGSIEEMLYSPDQTDEHVGTLSDRSKPILFSMARLDKVKNISGLVEMYSKNTKLRELVNLVVIAGNIDVNKSKDREEIVEIEKMHNLMKNYKLDGQFRWITAQTNRARNGELYRYIADTRGAFAQPAFYEAFGLTVVEAMTCGLPTFATCHGGPAEIIEHGLSGFHIDPYHPEQAGNIMADFFERCKEDPNHWKKVSDAGLQRIYERYTWKIYSERLMTLAGVYGFWKYVSKLERRETRRYLEMFYILKFRDLVKTVPSTADD

SEQ ID NO: 8 - protein, 805 aa, Vigna radiata
MATDRLTRVHSLRERLDETLSANRNEILALLSRIEGKGKGILQHHQVIAEFEEIPEESRQKLTDGAFGEVLRSTQEAIVLPPWVALAVRPRPGVWEYLRVNVHALVVEVLQPAEYLRFKEELVDGSSNGNFVLELDFEPFTASFPRPTLNKSIGNGVQFLNRHLSAKLFHDKESLHPLLEFLRLHSVKGKTLMLNDRIQNPDALQHVLRKAEEYLGTVPPETPYSAFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTENGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHTEIEELLYSSVENEEHICVLKDRSKPIIFTMARLDRVKNITGLVEWYGKNAKLRELVNLVVVAGDRRKESKDLEEKAEMKKMYSLIETYKLNGQFRWISSQMNRVRNGELYRVIADTKGAFVQPAVYEAFGLTVVEAMTCGLPTFATCNGGPAEIIVHGKSGFHIDPYHGDRAADLLVEFFEKVKVDPSHWDKISQAGLQRIEEKYTWQIYSQRLLTLTGVYGFWKHVSNLDRRESRRYLEMFYALKYRKLAESVPLAVE

SEQ ID NO: 9 - protein, 805 aa, Vigna radiata
MATDRLTRVHELRERLDETLSANRNEILALLSRIEGKGKGILQHHQVIAEFEEIPEESRQKLTDGAFGEVLRSTQEAIVLPPWVALAVRPRPGVWEYLRVNVHALVVEVLQPAEYLRFKEELVDGSSNGNFVLELDFEPFTASFPRPTLNKSIGNGVQFLNRHLSAKLFHDKESLHPLLEFLRLHSVKGKTLMLNDRIQNPDALQHVLRKAEEYLGTVPPETPYSAFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTENGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHTEIEELLYSSVENEEHICVLKDRSKPIIFTMARLDRVKNITGLVEWYGKNAKLRELVNLVVVAGDRRKESKDLEEKAEMKKMYSLIETYKLNGQFRWISSQMNRVRNGELYRVIADTKGAFVQPAVYEAFGLTVVEAMTCGLPTFATCNGGPAEIIVHGKSGFHIDPYHGDRAADLLVEFFEKVKVDPSHWDKISQAGLQRIEEKYTWQIYSQRLLTLTGVYGFWKHVSNLDRRESRRYLEMFYALKYRKLAESVPLAVE

SEQ ID NO: 10 - protein, 805 aa, Glycine max
MATDRLTRVHSLRERLDETLTANRNEILALLSRIEAKGKGILQHHQVIAEFEEIPEENRQKLTDGAFGEVLRSTQEAIVLPPWVALAVRPRPGVWEYLRVNVHALVVEELQPAEYLHFKEELVDGSSNGNFVLELDFEPFNAAFPRPTLNKSIGNGVQFLNRHLSAKLFHDKESLHPLLEFLRLHSVKGKTLMLNDRIQNPDALQHVLRKAEEYLGTVPPETPYSEFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTEKGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHPEIEELLYSSVENEEHICVLKDRSKPIIFTMARLDRVKNITGLVEWYGKNAKLRELVNLVVVAGDRRKESKDLEEKAEMKKMYGLIETYKLNGQFRWISSQMNRVRNGELYRVICDTRGAFVQPAVYEAFGLTVVEAMTCGLPTFATCNGGPAEIIVHGKSGFHIDPYHGDRAADLLVDFFEKCKLDPTHWDKISKAGLQRIEEKYTWQIYSQRLLTLTGVYGFWKHVSNLDRRESRRYLEMFYALKYRKLAESVPLAAE

SEQ ID NO: 11 - protein, 805 aa, Glycine max
MATDRLTRVHELRERLDETLTANRNEILALLSRIEAKGKGILQHHQVIAEFEEIPEENRQKLTDGAFGEVLRSTQEAIVLPPWVALAVRPRPGVWEYLRVNVHALVVEELQPAEYLHFKEELVDGSSNGNFVLELDFEPFNAAFPRPTLNKSIGNGVQFLNRHLSAKLFHDKESLHPLLEFLRLHSVKGKTLMLNDRIQNPDALQHVLRKAEEYLGTVPPETPYSEFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTEKGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHPEIEELLYSSVENEEHICVLKDRSKPIIFTMARLDRVKNITGLVEWYGKNAKLRELVNLVVVAGDRRKESKDLEEKAEMKKMYGLIETYKLNGQFRWISSQMNRVRNGELYRVICDTRGAFVQPAVYEAFGLTVVEAMTCGLPTFATCNGGPAEIIVHGKSGFHIDPYHGDRAADLLVDFFEKCKLDPTHWDKISKAGLQRIEEKYTWQIYSQRLLTLTGVYGFWKHVSNLDRRESRRYLEMFYALKYRKLAESVPLAAE

SEQ ID NO: 12 - protein, 807 aa, Anabaena sp. 54
MASELMQAILDSEEKHDLRGFISELRQQDKNYLLRNDILNVYAEYCSKCQKPETSYKFSNLSKLIYYTQEIIPEDSNFCFIIRPKIAAQEVYRLTADLDVEPMTVQELLDLRDRLVNKFHPYEGDILELDFGPFYDYTPTIRDPKNIGKGVQYLNRYLSSKLFQDSQQWLESLFNFLRLHNYNGIQLLINHQIQSQQQLSQQVKNALNFVSDRPNDEPYEQFRLQLQTMGFEPGWGNTASRVRDTLNILDELIDSPDPQTLEAFISRIPMIFRIVLVSAHGWFGQEGVLGRPDTGGQVVYVLDQAKNLEKQLQEDAILAGLEVLNVQPKVIILTRLIPNSDGTLCNQRLEKVYGTENAWILRVPLREFNPKMTQNWISRFEFWPYLETFAIDSERELLAEFQGRPDLIVGNYTDGNLVAFLLTRRMKVTQCNIAHALEKSKYLFSNLYWQDLEEKYHFSLQFTADLIAMNAANFVISSTYQEIVGTPDSIGQYESYKCFTMPELYHVVNGIELFSPKFNVVPPGVNENSYFPYTQTQNRIESDRDRLEEMLFTLEDSSQIFGKLDDPNKRPIFSMARLDRIKNLTGLAECFGQSQELQERCNLILVAGKLRIEESEDNEEKDEIVKLYRIIDEYNLHGKIRWLGVRLSKNDSGEIYRVICDRQGIFVQPALFEAFGLTILESMISGLPTFATQFGGPLEIIQDKINGFYINPTHLEETATKILDFVTKCEQNPNYWNIISEKAIDRVYSTYTWKIHTTKLLTLARIYGFWNFTSKEKREDLLRYLESLFYLIYKPRAQQLLEQHKYR

SEQ ID NO: 13 - protein, 463 aa, Artificial Sequence
  Synthetic Sequence
MATKGSSGMSLAERFWLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAEMIASIADERLEHAETESPAAAGQGRPAAAPTFEVARMKLIR

SEQ ID NO: 14 - protein, 463 aa, Artificial Sequence
  Synthetic Sequence
MATKNSSGMSLAERFWLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPITGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDSFHHWAAAAALEHKVPCAMMLLGSAEMIASIADERLEHAETESPAAAGQGRPAAAPTFEVARMKLIR

SEQ ID NO: 15 - protein, 465 aa, Artificial Sequence
  Synthetic Sequence
MARRTKNSSGMSLAERFWLTLSRSSLVVGRSCVEFEPETVPLLSTLRGKPITFLGLMPPLPEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPITGDQGPNARLIEAKNAGLQVARNDGDGSFDREGVAAAIRAVAVEEESSKVFQEKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDSFHHWAAAAALEHKVPCAMMFLGSAEMIASIADERLEHAETESPAAAGQGRPAAAPTFEVARMKLIR

SEQ ID NO: 16 - protein, 465 aa, Artificial Sequence
  Synthetic Sequence
MARRTKNSSGMSLAERFWLTLSRSSLVVGRSCVEFEPETVPLLSTLRGKPITFLGLMPPLPEGRREDGEDATVRWLDAQPAKSVVYVALGSEVPLGVEKVHELALGLELAGTRFLWALRKPTGVSDADLLPAGFEERTRGRGVVATRWVPQAAILAHAAVGAFLTHCGWNSTIEGLMFGHPLIMLPITGDQGPNARLIEAKNAGLQVARNDGDGSFDREGVAAAIRAVAVEEESSKVFQEKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDSFHHWAAAAALEHKVPCAMMFLGSAEMIASIADERLEHAETESPAAAGQGRPAAAPTFEVARMKLIR

SEQ ID NO: 17 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 18 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 19 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 20 - protein, 459 aa, Artificial Sequence
  Synthetic Sequence
MANWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 21 - protein, 459 aa, Stevia rebaudiana
MAENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 22 - protein, 459 aa, Artificial Sequence
  Synthetic Sequence
MAKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDS

SEQ ID NO: 23 - protein, 458 aa, Artificial Sequence
  Synthetic Sequence
MAFLWVVRPGFVKGSTWVEPLPDGFLGERGRIVKWVPQQEVLAHGAIGAFWTHGGWNSTLESVCEGVPMIFSDFGLDQPLNARYMSDVLKVGVYLENGWERGEIANAIRRLMVDEEGEYIRQNARVLKQKADVSLMKGGSSYESLESLVSYISSLGSGGSGGSGRRRRIILFPVPFQGHINPMLQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSQS

SEQ ID NO: 24 - protein, 455 aa, Artificial Sequence
  Synthetic Sequence
MAFLWVVRPGFVKGSTWVEPLPDGFLGERGRIVKWVPQQEVLAHGAIGAFWTHGGWNSTLESVCEGVPMIFSDFGLDQPLNARYMSDVLKVGVYLENGWERGEIANAIRRLMVDEEGEYIRQNARVLKQKADVSLMKGGSSYESLESLVSYISSLGSGGSGRRRRIILFPVPFQGHINPMLQLANVLYSKGFSITIFHTNFNKPKTSNYPHFTFRFILDNDPQDERISNLPTHGPLAGMRIPIINEHGADELRRELELQMLASEEDEEVSCLITDALWYFAQSVADSLNLPRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLEHDRTVFQWLDQQPPSSVLYVSFGSTSEVDEKDFLEIARGLVDSQS

SEQ ID NO: 25 - protein, 447 aa, Artificial Sequence
  Synthetic Sequence
MAFLWVVRPGFVKGSTWVEPLPDGFLGERGRIVKWVPQQEVLAHGAIGAFWTHGGWNSTLESVCEGVPMIFQDFGLDQPLNARYMSDVLKVGVYLENGWERGEIANAIRRLMVDEEGEYIRQNARVLKQKADVSLMKGGSSYESLESLVSYISSLGSGGSGRRRRIILFPVPFQGHINPMLQLANVLYSKGFSITIFHTNFNKPKTSNYPHFTFRFILDNDPQDHGPLAGMRIPIINEHGADELRRELELQMLASEEDEEVSCLITDALWYFAQSVADSLNLPRLVLMTSSLFNFHCHVSLPQFDELGYLDPDDKTRLEEQASGFPMLKVKDIKSAFSNWQIAKEILGKMIKQTKASSGVIWNSFKELEESELETVIREIPAPSFLIPLPKHLTASSSSLLEHDRTVFQWLDQQPPSSVIYVSFGSTSEVDEKDFLEIARGLVDSQS

SEQ ID NO: 26 - protein, 485 aa, Stevia rebaudiana
MYNVTYHQNSKAMATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 27 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 28 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 29 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 30 - protein, 463 aa, Artificial Sequence
  Synthetic Sequence
MAECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKAGSGEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHH

SEQ ID NO: 31 - protein, 463 aa, Artificial Sequence
  Synthetic Sequence
MANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKAGSGEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKA

SEQ ID NO: 32 - protein, 482 aa, Stevia rebaudiana
MADAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEATQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 33 - protein, 523 aa, Siraitia grosvenorii
MEDRNAMDMSRIKYRPQPLRPASMVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGINLHPTISLKKDTPTTSEHPRHEFENMRGMNYEMLVGNAIKSPTLTKK

SEQ ID NO: 34 - protein, 473 aa, Siraitia grosvenorii
MTIFFSVEILVLGIAEFAAIAMDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 35 - protein, 460 aa, Siraitia grosvenorii
MDETTVNGGRRASDVVVFAFPRHGHMSPMLQFSKRLVSKGLRVTFLITTSATESLRLNLPPSSSLDLQVISDVPESNDIATLEGYLRSFKATVSKTLADFIDGIGNPPKFIVYDSVMPWVQEVARGRGLDAAPFFTQSSAVNHILNHVYGGSLSIPAPENTAVSLPSMPVLQAEDLPAFPDDPEVVMNFMTSQFSNFQDAKWIFFNTFDQLECKKQSQVVNWMADRWPIKTVGPTIPSAYLDDGRLEDDRAFGLNLLKPEDGKNTRQWQWLDSKDTASVLYISFGSLAILQEEQVKELAYFLKDTNLSFLWVLRDSELQKLPHNFVQETSHRGLVVNWCSQLQVLSHRAVSCFVTHCGWNSTLEALSLGVPMVAIPQWVDQTTNAKFVADVWRVGVRVKKKDERIVTKEELEASIRQVVQGEGRNEFKHNAIKWKKLAKEAVDEGGSSDKNIEEFVKTIA

SEQ ID NO: 36 - protein, 486 aa, Siraitia grosvenorii
MGDNGDGGEKKELKENVKKGKELGRQAIGEGYINPSLQLARRLISLGVNVTFATTVLAGRRMKNKTHQTATTPGLSFATFSDGFDDETLKPNGDLTHYFSELRRCGSESLTHLITSAANEGRPITFVIYSLLLSWAADIASTYDIPSALFFAQPATVLALYFYYFHGYGDTICSKLQDPSSYIELPGLPLLTSQDMPSFFSPSGPHAFILPPMREQAEFLGRQSQPKVLVNTFDALEADALRAIDKLKMLAIGPLIPSALLGGNDSSDASFCGDLFQVSSEDYIEWLNSKPDSSVVYISVGSICVLSDEQEDELVHALLNSGHTFLWVKRSKENNEGVKQETDEEKLKKLEEQGKMVSWCRQVEVLKHPALGCFLTHCGWNSTIESLVSGLPVVAFPQQIDQATNAKLIEDVWKTGVRVKANTEGIVEREEIRRCLDLVMGSRDGQKEEIERNAKKWKELARQAIGEGGSSDSNLKTFLWEIDLEI

SEQ ID NO: 37 - protein, 485 aa, Siraitia grosvenorii
MAEQAHDLLHVLLFPFPAEGHIKPFLCLAELLCNAGFHVTFLNTDYNHRRLHNLHLLAARFPSLHFESISDGLPPDQPRDILDPKFFISICQVTKPLFRELLLSYKRISSVQTGRPPITCVITDVIFRFPIDVAEELDIPVFSFCTFSARFMFLYFWIPKLIEDGQLPYPNGNINQKLYGVAPEAEGLLRCKDLPGHWAFADELKDDQLNFVDQTTASSRSSGLILNTFDDLEAPFLGRLSTIFKKIYAVGPIHSLLNSHHCGLWKEDHSCLAWLDSRAAKSVVFVSFGSLVKITSRQLMEFWHGLLNSGKSFLFVLRSDVVEGDDEKQVVKEIYETKAEGKWLVVGWAPQEKVLAHEAVGGFLTHSGWNSILESIAAGVPMISCPKIGDQSSNCTWISKVWKIGLEMEDRYDRVSVETMVRSIMEQEGEKMQKTIAELAKQAKYKVSKDGTSYQNLECLIQDIKKLNQIEGFINNPNFSDLLRV

SEQ ID NO: 38 - protein, 473 aa, Siraitia grosvenorii
MDAQQGHTTTILMLPWVGYGHLLPFLELAKSLSRRKLFHIYFCSTSVSLDAIKPKLPPSISSDDSIQLVELRLPSSPELPPHLHTTNGLPSHLMPALHQAFVMAAQHFQVILQTLAPHLLIYDILQPWAPQVASSLNIPAINFSTTGASMLSRTLHPTHYPSSKFPISEFVLHNHWRAMYTTADGALTEEGHKIEETLANCLHTSCGVVLVNSFRELETKYIDYLSVLLNKKVVPVGPLVYEPNQEGEDEGYSSIKNWLDKKEPSSTVFVSFGTEYFPSKEEMEEIAYGLELSEVNFIWVLRFPQGDSTSTIEDALPKGFLERAGERAMVVKGWAPQAKILKHWSTGGLVSHCGWNSMMEGMMFGVPIIAVPMHLDQPFNAGLVEEAGVGVEAKRDSDGKIQREEVAKSIKEVVIEKTREDVRKKAREMDTKHGPTYFSRSKVSSFGRLYKINRPTTLTVGRFWSKQIKMKRE

SEQ ID NO: 39 - protein, 453 aa, Siraitia grosvenorii
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 40 - protein, 472 aa, Momordica charantia
MAQPQTQARVLVFPYPTVGHIKPFLSLAELLADGGLDVVFLSTEYNHRRIPNLEALASRFPTLHFDTIPDGLPIDKPRVIIGGELYTSMRDGVKQRLRQVLQSYNDGSSPITCVICDVMLSGPIEAAEELGIPVVTFCPYSARYLCAHFVMPKLIEEGQIPFTDGNLAGEIQGVPLFGGLLRRDHLPGFWFVKSLSDEVWSHAFLNQTLAVGRTSALIINTLDELEAPFLAHLSSTFDKIYPIGPLDALSKSRLGDSSSSSTVLTAFWKEDQACMSWLDSQPPKSVIFVSFGSTMRMTADKLVEFWHGLVNSGTRFLCVLRSDIVEGGGAADLIKQVGETGNGIVVEWAAQEKVLAHRAVGGFLTHCGWNSTMESIAAGVPMMCWQIYGDQMINATWIGKVWKIGIERDDKWDRSTVEKMIKELMEGEKGAEIQRSMEKFSKLANDKVVKGGTSFENLELIVEYLKKLKPSN

SEQ ID NO: 41 - protein, 466 aa, Momordica charantia
MAQPRVLLFPFPAMGHVKPFLSLAELLSDAGVEVVFLSTEYNHRRIPDIGALAARFPTLHFETIPDGLPPDQPRVLADGHLYFSMLDGTKPRFRQLIQSLNGNPRPITCIINDVMLSSPIEVAEEFGIPVIAFCPCSARFLSVHFFMPNFIEEAQIPYTDENPMGKIEEATVFEGLLRRKDLPGLWCAKSSNISFSHRFINQTIAAGRASALILNTFDELESPFLNHLSSIFPKIYCIGPLNALSRSRLGKSSSSSSALAGFWKEDQAYMSWLESQPPRSVIFVSFGSTMKMEAWKLAEFWYGLVNSGSPFLFVFRPDCVINSGDAAEVMEGRGRGMVVEWASQEKVLAHPAVGGFLTHCGWNSTVESIVAGVPMMCCPIVADQLSNATWIHKVWKIGIEGDEKWDRSTVEMMIKELMESQKGTEIRTSIEMLSKLANEKVVKGGTSLNNFELLVEDIKTLRRPYT

SEQ ID NO: 42 - protein, 482 aa, Momordica charantia
MEQSDSNSDDHQHHVLLFPFPAKGHIKPFLCLAQLLCGAGLQVTFLNTDHNHRRIDDRHRRLLATQFPMLHFKSISDGLPPDHPRDLLDGKLIASMRRVTESLFRQLLLSYNGYGNGTNNVSNSGRRPPISCVITDVIFSFPVEVAEELGIPVFSFATFSARFLFLYFWIPKLIQEGQLPFPDGKTNQELYGVPGAEGIIRCKDLPGSWSVEAVAKNDPMNFVKQTLASSRSSGLILNTFEDLEAPFVTHLSNTFDKIYTIGPIHSLLGTSHCGLWKEDYACLAWLDARPRKSVVFVSFGSLVKTTSRELMELWHGLVSSGKSFLLVLRSDVVEGEDEEQVVKEILESNGEGKWLVVGWAPQEEVLAHEAIGGFLTHSGWNSTMESIAAGVPMVCWPKIGDQPSNCTWVSRVWKVGLEMEERYDRSTVARMARSMMEQEGKEMERRIAELAKRVKYRVGKDGESYRNLESLIRDIKITKSSN

SEQ ID NO: 43 - protein, 450 aa, Momordica charantia
MDAHQQAEHTTTILMLPWVGYGHLTAYLELAKALSRRNFHIYYCSTPVNIESIKPKLTIPCSSIQFVELHLPSSDDLPPNLHTTNGLPSHLMPTLHQAFSAAAPLFEEILQTLCPHLLIYDSLQPWAPKIASSLKIPALNFNTSGVSVIAQALHAIHHPDSKFPLSDFILHNYWKSTYTTADGGASEKTRRAREAFLYCLNSSGNAILINTFRELEGEYIDYLSLLLNKKVIPIGPLVYEPNQDEDQDEEYRSIKNWLDKKEPCSTVFVSFGSEYFPSNEEMEEIAPGLEESGANFIWVVRFPKLENRNGIIEEGLLERAGERGMVIKEWAPQARILRHGSIGGFVSHCGWNSVMESIICGVPVIGVPMRVDQPYNAGLVEEAGVGVEAKRDPDGKIQRHEVSKLIKQVVVEKTRDDVRKKVAQMSEILRRKGDEKIDEMVALISLLPKG

SEQ ID NO: 44 - protein, 450 aa, Momordica charantia
MDARQQAEHTTTILMLPWVGYGHLSAYLELAKALSRRNFHIYYCSTPVNIESIKPKLTIPCSSIQFVELHLPFSDDLPPNLHTTNGLPSHLMPALHQAFSAAAPLFEAILQTLCPHLLIYDSLQPWAPQIASSLKIPALNFNTTGVSVIARALHTIHHPDSKFPLSEIVLHNYWKATHATADGANPEKFRRDLEALLCCLHSSCNAILINTFRELEGEYIDYLSLLLNKKVTPIGPLVYEPNQDEEQDEEYRSIKNWLDKKEPYSTIFVSFGSEYFPSNEEMEEIARGLEESGANFIWVVRFHKLENGNGITEEGLLERAGERGMVIQGWAPQARILRHGSIGGFVSHCGWNSVMESIICGVPVIGVPMGLDQPYNAGLVEEAGVGVEAKRDPDGKIQRHEVSKLIKQVVVEKTRDDVRKKVAQMSEILRRKGDEKIDEMVALISLLLKG

SEQ ID NO: 45 - protein, 471 aa, Cucumis sativus
MGLSPTDHVLLFPFPAKGHIKPFFCLAHLLCNAGLRVTFLSTEHHHQKLHNLTHLAAQIPSLHFQSISDGLSLDHPRNLLDGQLFKSMPQVTKPLFRQLLLSYKDGTSPITCVITDLILRFPMDVAQELDIPVFCFSTFSARFLFLYFSIPKLLEDGQIPYPEGNSNQVLHGIPGAEGLLRCKDLPGYWSVEAVANYNPMNFVNQTIATSKSHGLILNTFDELEVPFITNLSKIYKKVYTIGPIHSLLKKSVQTQYEFWKEDHSCLAWLDSQPPRSVMFVSFGSIVKLKSSQLKEFWNGLVDSGKAFLLVLRSDALVEETGEEDEKQKELVIKEIMETKEEGRWVIVNWAPQEKVLEHKAIGGFLTHSGWNSTLESVAVGVPMVSWPQIGDQPSNATWLSKVWKIGVEMEDSYDRSTVESKVRSIMEHEDKKMENAIVELAKRVDDRVSKEGTSYQNLQRLIEDIEGFKLN

SEQ ID NO: 46 - protein, 462 aa, Cucurbita maxima
MELSHTHHVLLFPFPAKGHIKPFFSLAQLLCNAGLRVTFLNTDHHHRRIHDLNRLAAQLPTLHFDSVSDGLPPDEPRNVFDGKLYESIRQVTSSLFRELLVSYNNGTSSGRPPITCVITDVMFRFPIDIAEELGIPVFTFSTFSARFLFLIFWIPKLLEDGQLRYPEQELHGVPGAEGLIRWKDLPGFWSVEDVADWDPMNFVNQTLATSRSSGLILNTFDELEAPFLTSLSKIYKKIYSLGPINSLLKNFQSQPQYNLWKEDHSCMAWLDSQPRKSVVFVSFGSVVKLTSRQLMEFWNGLVNSGMPFLLVLRSDVIEAGEEVVREIMERKAEGRWVIVSWAPQEEVLAHDAVGGFLTHSGWNSTLESLAAGVPMISWPQIGDQTSNSTWISKVWRIGLQLEDGFDSSTIETMVRSIMDQTMEKTVAELAERAKNRASKNGTSYRNFQTLIQDITNIIETHI

SEQ ID NO: 47 - protein, 474 aa, Prunus persica
MAMKQPHVIIFPFPLQGHMKPLLCLAELLCHAGLHVTYVNTHHNHQRLANRQALSTHFPTLHFESISDGLPEDDPRTLNSQLLIALKTSIRPHFRELLKTISLKAESNDTLVPPPSCIMTDGLVTFAFDVAEELGLPILSFNVPCPRYLWTCLCLPKLIENGQLPFQDDDMNVEITGVPGMEGLLHRQDLPGFCRVKQADHPSLQFAINETQTLKRASALILDTVYELDAPCISHMALMFPKIYTLGPLHALLNSQIGDMSRGLASHGSLWKSDLNCMTWLDSQPSKSIIYVSFGTLVHLTRAQVIEFWYGLVNSGHPFLWVMRSDITSGDHQIPAELENGTKERGCIVDWVSQEEVLAHKSVGGFLTHSGWNSTLESIVAGLPMICWPKLGDHYIISSTVCRQWKIGLQLNENCDRSNIESMVQTLMGSKREEIQSSMDAISKLSRDSVAEGGSSHNNLEQLIEYIRNLQHQN

SEQ ID NO: 48 - protein, 468 aa, Theobroma cacao
MRQPHVLVLPFPAQGHIKPMLCLAELLCQAGLRVTFLNTHHSHRRLNNLQDLSTRFPTLHFESVSDGLPEDHPRNLVHFMHLVHSIKNVTKPLLRDLLTSLSLKTDIPPVSCIIADGILSFAIDVAEELQIKVIIFRTISSCCLWSYLCVPKLIQQGELQFSDSDMGQKVSSVPEMKGSLRLHDRPYSFGLKQLEDPNFQFFVSETQAMTRASAVIFNTFDSLEAPVLSQMIPLLPKVYTIGPLHALRKARLGDLSQHSSFNGNLREADHNCITWLDSQPLRSVVYVSFGSHVVLTSEELLEFWHGLVNSGKRFLWVLRPDIIAGEKDHNQIIAREPDLGTKEKGLLVDWAPQEEVLAHPSVGGFLTHCGWNSTLESMVAGVPMLCWPKLPDQLVNSSCVSEVWKIGLDLKDMCDRSTVEKMVRALMEDRREEVMRSVDGISKLARESVSHGGSSSSNLEMLIQELET

SEQ ID NO: 49 - protein, 454 aa, Cucurbita maxima
MDAQKAVDTPPTTVLMLPWIGYGHLSAYLELAKALSRRNFHVYFCSTPVNLDSIKPNLIPPPSSIQFVDLHLPSSPELPPHLHTTNGLPSHLKPTLHQAFSAAAQHFEAILQTLSPHLLIYDSLQPWAPRIASSLNIPAINFNTTAVSIIAHALHSVHYPDSKFPFSDFVLHDYWKAKYTTADGATSEKIRRGAEAFLYCLNASCDVVLVNSFRELEGEYMDYLSVLLKKKVVSVGPLVYEPSEGEEDEEYWRIKKWLDEKEALSTVLVSFGSEYFPSKEEMEEIAHGLEESEANFIWVVRFPKGEESCRGIEEALPKGFVERAGERAMVVKKWAPQGKILKHGSIGGFVSHCGWNSVLESIRFGVPVIGVPMHLDQPYNAGLLEEAGIGVEAKRDADGKIQRDQVASLIKRVVVEKTREDIWKTVREMREVLRRRDDDMIDEMVAEISVVLKI

SEQ ID NO: 50 - protein, 457 aa, Cucurbita moschata
MDAQKAVDTPPTTVLMLPWIGYGHLSAYLELAKALSRRNFHVYFCSTPVNLDSIKPNLIPPPPSIQFVDLHLPSSPELPPHLHTTNGLPSHLKPTLHQAFSAAAQHFEAILQTLSPHLLIYDSLQPWAPRIASSLNIPAINFNTTAVSIIAHALHSVHYPDSKFPFSDFVLHDYWKAKYTTADGATSEKTRRGVEAFLYCLNASCDVVLVNSFRELEGEYMDYLSVLLKKKVVSVGPLVYEPSEGEEDEEYWRIKKWLDEKEALSTVLVSFGSEYFPPKEEMEEIAHGLEESEANFIWVVRFPKGEESSSRGIEEALPKGFVERAGERAMVVKKWAPQGKILKHGSIGGFVSHCGWNSVLESIRFGVPVIGAPMHLDQPYNAGLLEEAGIGVEAKRDADGKIQRDQVASLIKQVVVEKTREDIWKKVREMREVLRRRDDDDMMIDEMVAVISVVLKI

SEQ ID NO: 51 - protein, 492 aa, Cucurbita maxima
MSSNLFLKISIPFGRLRDSALNCSVFHCKLHLAIAIAMDAQQAANKSPTATTIFMLPWAGYGHLSAYLELAKALSTRNFHIYFCSTPVSLASIKPRLIPSCSSIQFVELHLPSSDEFPPHLHTTNGLPSRLVPTFHQAFSEAAQTFEAFLQTLRPHLLIYDSLQPWAPRIASSLNIPAINFFTAGAFAVSHVLRAFHYPDSQFPSSDFVLHSRWKIKNTTAESPTQAKLPKIGEAIGYCLNASRGVILTNSFRELEGKYIDYLSVILKKRVFPIGPLVYQPNQDEEDEDYSRIKNWLDRKEASSTVLVSFGSEFFLSKEETEAIAHGLEQSEANFIWGIRFPKGAKKNAIEEALPEGFLERAGGRAMVVEEWVPQGKILKHGSIGGFVSHCGWNSAMESIVCGVPIIGIPMQVDQPFNAGILEEAGVGVEAKRDSDGKIQRDEVAKLIKEVVVERTREDIRNKLEKINEILRSRREEKLDELATEISLLSRN

SEQ ID NO: 52 - protein, 455 aa, Cucurbita moschata
MDAQQAANKSPTASTIFMLPWVGYGHLSAYLELAKALSTRNFHVYFCSTPVSLASIKPRLIPSCSSIQFVELHLPSSDEFPPHLHTTNGLPAHLVPTIHQAFAAAAQTFEAFLQTLRPHLLIYDSLQPWAPRIASSLNIPAINFFTAGAFAVSHVLRAFHYPDSQFPSSDFVLHSRWKIKNTTAESPTQVKIPKIGEAIGYCLNASRGVILTNSFRELEGKYIDYLSVILKKRVLPIGPLVYQPNQDEEDEDYSRIKNWLDRKEASSTVLVSFGSEFFLSKEETEAIAHGLEQSEANFIWGIRFPKGAKKNAIEEALPEGFLERVGGRAMVVEEWVPQGKILKHGNIGGFVSHCGWNSAMESIMCGVPVIGIPMQVDQPFNAGILEEAGVGVEAKRDSDGKIQRDEVAKLIKEVVVERTREDIRNKLEEINEILRTRREEKLDELATEISLLCKN

SEQ ID NO: 53 - protein, 462 aa, Corchorus capsularis
MDSKQKKMSVLMFPWLAYGHISPFLELAKKLSKRNFHTFFFSTPINLNSIKSKLSPKYAQSIQFVELHLPSLPDLPPHYHTTNGLPPHLMNTLKKAFDMSSLQFSKILKTLNPDLLVYDFIQPWAPLLALSNKIPAVHFLCTSAAMSSFSVHAFKKPCEDFPFPNIYVHGNFMNAKFNNMENCSSDDSISDQDRVLQCFERSTKIILVKTFEELEGKFMDYLSVLLNKKIVPTGPLTQDPNEDEGDDDERTKLLLEWLNKKSKSSTVFVSFGSEYFLSKEEREEIAYGLELSKVNFIWVIRFPLGENKTNLEEALPQGFLQRVSERGLVVENWAPQAKILQHSSIGGFVSHCGWSSVMESLKFGVPIIAIPMHLDQPLNARLVVDVGVGLEVIRNHGSLEREEIAKLIKEVVLGNGNDGEIVRRKAREMSNHIKKKGEKDMDELVEELMLICKMKPNSCHLS

SEQ ID NO: 54 - protein, 448 aa, Ziziphus jujube
MMERQRSIKVLMFPWLAHGHISPFLELAKRLTDRNFQIYFCSTPVNLTSVKPKLSQKYSSSIKLVELHLPSLPDLPPHYHTTNGLALNLIPTLKKAFDMSSSSFSTILSTIKPDLLIYDFLQPWAPQLASCMNIPAVNFLSAGASMVSFVLHSIKYNGDDHDDEFLTTELHLSDSMEAKFAEMTESSPDEHIDRAVTCLERSNSLILIKSFRELEGKYLDYLSLSFAKKVVPIGPLVAQDTNPEDDSMDIINWLDKKEKSSTVFVSFGSEYYLTNEEMEEIAYGLELSKVNFIWVVRFPLGQKMAVEEALPKGFLERVGEKGMVVEDWAPQMKILGHSSIGGFVSHCGWSSLMESLKLGVPIIAMPMQLDQPINAKLVERSGVGLEVKRDKNGRIEREYLAKVIREIVVEKARQDIEKKAREMSNIITEKGEEEIDNVVEELAKLCGM

SEQ ID NO: 55 - protein, 446 aa, Vitis vinifera
MDARQSDGISVLMFPWLAHGHISPFLQLAKKLSKRNFSIYFCSTPVNLDPIKGKLSESYSLSIQLVKLHLPSLPELPPQYHTTNGLPPHLMPTLKMAFDMASPNFSNILKTLHPDLLIYDFLQPWAPAAASSLNIPAVQFLSTGATLQSFLAHRHRKPGIEFPFQEIHLPDYEIGRLNRFLEPSAGRISDRDRANQCLERSSRFSLIKTFREIEAKYLDYVSDLTKKKMVTVGPLLQDPEDEDEATDIVEWLNKKCEASAVFVSFGSEYFVSKEEMEEIAHGLELSNVDFIWVVRFPMGEKIRLEDALPPGFLHRLGDRGMVVEGWAPQRKILGHSSIGGFVSHCGWSSVMEGMKFGVPIIAMPMHLDQPINAKLVEAVGVGREVKRDENRKLEREEIAKVIKEVVGEKNGENVRRKARELSETLRKKGDEEIDVVVEELKQLCSY

SEQ ID NO: 56 - protein, 456 aa, Juglans regia
MDTARKRIRVVMLPWLAHGHISPFLELSKKLAKRNFHIYFCSTPVNLSSIKPKLSGKYSRSIQLVELHLPSLPELPPQYHTTKGLPPHLNATLKRAFDMAGPHFSNILKTLSPDLLIYDFLQPWAPAIAASQNIPAINFLSTGAAMTSFVLHAMKKPGDEFPFPEIHLDECMKTRFVDLPEDHSPSDDHNHISDKDRALKCFERSSGFVMMKTFEELEGKYINFLSHLMQKKIVPVGPLVQNPVRGDHEKAKTLEWLDKRKQSSAVFVSFGTEYFLSKEEMEEIAYGLELSNVNFIWVVRFPEGEKVKLEEALPEGFLQRVGEKGMVVEGWAPQAKILMHPSIGGFVSHCGWSSVMESIDFGVPIVAIPMQLDQPVNAKVVEQAGVGVEVKRDRDGKLEREEVATVIREVVMGNIGESVRKKEREMRDNIRKKGEEKMDGVAQELVQLYGNGIKNV

SEQ ID NO: 57 - protein, 456 aa, Hevea brasiliensis
METLQRRKISVLMFPWLAHGHLSPFLELSKKLNKRNFHVYFCSTPVNLDSIKPKLSAEYSFSIQLVELHLPSSPELPLHYHTTNGLPPHLMKNLKNAFDMASSSFFNILKTLKPDLLIYDFIQPWAPALASSLNIPAVNFLCTSMAMSCFGLHLNNQEAKFPFPGIYPRDYMRMKVFGALESSSNDIKDGERAGRCMDQSFHLILAKTFRELEGKYIDYLSVKLMKKIVPVGPLVQDPIFEDDEKIMDHHQVIKWLEKKERLSTVFVSFGTEYFLSTEEMEEIAYGLELSKAHFIWVVRFPTGEKINLEESLPKRYLERVQERGKIVEGWAPQQKILRHSSIGGFVSHCGWSSIMESMKFGVPIIAMPMNLDQPVNSRIVEDAGVGIEVRRNKSGELEREEIAKTIRKVVVEKDGKNVSRKAREMSDTIRKKGEEEIDGVVDELLQLCDVKTNYLQ

SEQ ID NO: 58 - protein, 461 aa, Manihot esculenta
MATAQTRKISVLMFPWLAHGHLSPFLELSKKLANRNFHVYFCSTPVNLDSIKPKLSPEYHFSIQFVELHLPSSPELPSHYHTTNGLPPHLMKTLKKAFDMASSSFFNILKTLNPDLLIYDFLQPWAPALASSLNIPAVNFLCSSMAMSCFGLNLNKNKEIKFLFPEIYPRDYMEMKLFRVFESSSNQIKDGERAGRCIDQSFHVILAKTFRELEGKYIDYVSVKCNKKIVPVGPLVEDTIHEDDEKTMDHHHHHHDEVIKWLEKKERSTTVFVSFGSEYFLSKEEMEEIAHGLELSKVNFIWVVRFPKGEKINLEESLPEGYLERIQERGKIVEGWAPQRKILGHSSIGGFVSHCGWSSIMESMKLGVPIIAMPMNLDQPINSRIVEAAGVGIEVSRNQSGELEREEMAKTIRKVVVEREGVYVRRKAREMSDVLRKKGEEEIDGVVDELVQLCDMKTNYL

SEQ ID NO: 59 - protein, 452 aa, Cephalotus follicularis
MDLKRRSIRVLMLPWLAHGHISPFLELAKKLTNRNFLIYFCSTPINLNSIKPKLSSKYSFSIQLVELHLPSLPELPPHYHTTNGLPLHLMNTLKTAFDMASPSFLNILKTLKPDLLICDHLQPWAPSLASSLNIPAIIFPTNSAIMMAFSLHHAKNPGEEFPFPSININDDMVKSINFLHSASNGLTDMDRVLQCLERSSNTMLLKTFRQLEAKYVDYSSALLKKKIVLAGPLVQVPDNEDEKIEIIKWLDSRGQSSTVFVSFGSEYFLSKEEREDIAHGLELSKVNFIWVVRFPVGEKVKLEEALPNGFAERIGERGLVVEGWAPQAMILSHSSIGGFVSHCGWSSMMESMKFGVPIIAMPMHIDQPLNARLVEDVGVGLEIKRNKDGRFEREELARVIKEVLVYKNGDAVRSKAREMSEHIKKNGDQEIDGVADALVKLCEMKTNSLNQD

SEQ ID NO: 60 - protein, 451 aa, Coffea arabica
MENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNAIFYRDYESVFVENLTRRDRDTYRVINCMERSSKIILIKGFNEIEGKYFDYFSCLTGKKVVPVGPLVQDPVLDDEDCRIMQWLNKKEKGSTVFVSFGSEYFLSKKDMEEIAHGLEVSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVMKEASGESVRKKARELQEKLELKGDEEIDDVVKELVQLCATKNKRNGLHYY

SEQ ID NO: 61 - protein, 461 aa, Cucurbita moschata
MELSPTHHLLLFPFPAKGHIKPFFSLAQLLCNAGARVTFLNTDHHHRRIHDLDRLAAQLPTLHFDSVSDGLPPDESRNVFDGKLYESIRQVTSSLFRELLVSYNNGTSSGRPPITCVITDCMFRFPIDIAEELGIPVFTFSTFSARFLFLFFWIPKLLEDGQLRYPEQELHGVPGAEGLIRCKDLPGFLSDEDVAHWKPINFVNQILATSRSSGLILNTFDELEAPFLTSLSKIYKKIYSLGPINSLLKNFQSQPQYNLWKEDHSCMAWLDSQPPKSVVFVSFGSVVKLTNRQLVEFWNGLVNSGKPFLLVLRSDVIEAGEEVVRENMERKAEGRWMIVSWAPQEEVLAHDAVGGFLTHSGWNSTLESLAAGVPMISWTQIGDQTSNSTWVSKVWRIGLQLEDGFDSFTIETMVRSVMDQTMEKTVAELAERAKNRASKNGTSYRNFQTLIQDITNIIETH

SEQ ID NO: 62 - protein, 490 aa, Arabidopsis thaliana
MGSISEMVFETCPSPNPIHVMLVSFQGQGHVNPLLRLGKLIASKGLLVTFVTTELWGKKMRQANKIVDGELKPVGSGSIRFEFFDEEWAEDDDRRADFSLYIAHLESVGIREVSKLVRRYEEANEPVSCLINNPFIPWVCHVAEEFNIPCAVLWVQSCACFSAYYHYQDGSVSFPTETEPELDVKLPCVPVLKNDEIPSFLHPSSRFTGFRQAILGQFKNLSKSFCVLIDSFDSLEREVIDYMSSLCPVKTVGPLFKVARTVTSDVSGDICKSTDKCLEWLDSRPKSSVVYISFGTVAYLKQEQIEEIAHGVLKSGLSFLWVIRPPPHDLKVETHVLPQELKESSAKGKGMIVDWCPQEQVLSHPSVACFVTHCGWNSTMESLSSGVPVVCCPQWGDQVTDAVYLIDVFKTGVRLGRGATEERVVPREEVAEKLLEATVGEKAEELRKNALKWKAEAEAAVAPGGSSDKNFREFVEKLGAGVTKTKDNGY

SEQ ID NO: 63 - protein, 481 aa, Arabidopsis thaliana
MGSHVAQKQHVVCVPYPAQGHINPMMKVAKLLYAKGFHITFVNTVYNHNRLLRSRGPNAVDGLPSFRFESIPDGLPETDVDVTQDIPTLCESTMKHCLAPFKELLRQINARDDVPPVSCIVSDGCMSFTLDAAEELGVPEVLFWTTSACGFLAYLYYYRFIEKGLSPIKDESYLTKEHLDTKIDWIPSMKNLRLKDIPSFIRTTNPDDIMLNFIIREADRAKRASAIILNTFDDLEHDVIQSMKSIVPPVYSIGPLHLLEKQESGEYSEIGRTGSNLWREETECLDWLNTKARNSVVYVNFGSITVLSAKQLVEFAWGLAATGKEFLWVIRPDLVAGDEAMVPPEFLTATADRRMLASWCPQEKVLSHPAIGGFLTHCGWNSTLESLCGGVPMVCWPFFAEQQTNCKFSRDEWEVGIEIGGDVKREEVEAVVRELMDEEKGKNMREKAEEWRRLANEATEHKHGSSKLNFEMLVNKVLLGE

SEQ ID NO: 64 - protein, 438 aa, Columba livia
MIHCGKKHICAFVTCILISASILMYSWKDPQLQNNITRKIFQATSALPASQLCRGKPAQNVITALEDNRTFIISPYFDDRESKVTRVIGIVHHEDVKQLYCWFCCQPDGKIYVARAKIDVHSDRFGFPYGAADIVCLEPENCNPTHVSIHQSPHANIDQLPSFKIKNRKSETFSVDFTVCISAMFGNYNNVLQFIQSVEMYKILGVQKVVIYKNNCSQLMEKVLKFYMEEGTVEIIPWPINSHLKVSTKWHFSMDAKDIGYYGQITALNDCIYRNMQRSKFVVLNDADEIILPLKHLDWKAMMSSLQEQNPGAGIFLFENHIFPKTVSTPVFNISSWNRVPGVNILQHVHREPDRKEVFNPKKMIIDPRQVVQTSVHSVLRAYGNSVNVPADVALVYHCRVPLQEELPRESLIRDTALWRYNSSLITNVNKVLHQTVL

SEQ ID NO: 65 - protein, 252 aa, Haemophilus ducreyi
MPTLTVAMIVKNEAQDLAECLKTVDGWVDEIVIVDSGSTDDTLKIATQFNAKVYVNSDWQGFGPQRQFAQQYVTSDYVLWLDADERVTPELKASILQAVQHNQKNTVYKVSRLSEIFGKEIRYSGWYPDYVVRLYPTYLAKYGDELVHEKVHYPADSRVEKLQGDLLHFTYKNIHHYLVKSASYAKAWAMQRAKAGKKASLLDGVTHAIACFLKMYLFKAGFLDGKQGFLLAVLSAHSTFVKYADLWDRTRS

SEQ ID NO: 66 - protein, 252 aa, Neisseria gonorrhoeae
MKKVSVLIVAKNEANHIRECIESCRFDKEVIVIDDHSADNTAEIAEGLGAKVFRRHLNGDFGAQKTFAIEQAGGEWVFLIDADERCTPELSDEISKIVRTGDYAAYFVERRNLFPNHPATHGAMRPDSVCRLMPKKGGSVQGKVHETVQTPYPERRLKHFMYHYTYDNWEQYFNKFNKYTSISAEKYREQGKPVSFVRDIILRPIWGFFKIYILNKGFLDGKMGWIMSVNHSYYTMIKYVKLYYLYKSGGKF

SEQ ID NO: 67 - protein, 309 aa, Rhizobium meliloti
MPNETLHIDIGVCTYRRPELAETLRSLAAMNVPERARLRVIVADNDAEPSARALVEGLRPEMPFDILYVHCPHSNISIARNCCLDNSTGDFLAFLDDDETVSGDWLTRLLETARTTGAAAVLGPVRAHYGPTAPRWMRSGDFHSTLPVWAKGEIRTGYTCNALLRRDAASLLGRRFKLSLGKSGGEDTDFFTGMHCAGGTIAFSPEAWVHEPVPENRASLAWLAKRRFRSGQTHGRLLAEKAHGLRQAWNIALAGAKSGFCATAAVLCFPSAARRNRFALRAVLHAGVISGLLGLKEIEQYGAREVTSA

SEQ ID NO: 68 - protein, 861 aa, Rhizobium radiobacter
MCRCGRAVRSRPVCRPGQLVVRRSPRPRSRNHSRCRPLRLSVFPRPHRRVRHHCQRDLRWEPGRWIAVRWKAARSHRRFRRCPFPRQLVWPVRERHRDAGDRRNQRERRRRDAYHEISEPKFRTRKRTESFWMNKAITVIVWLLVSLCVLAIITMPVSLQTHLVATAISLILLATIKSFNGQGAWRLVALGFGTAIVLRYVYWRTTSTLPPVNQLENFIPGFLLYLAEMYSVVMLGLSLVIVSMPLPSRKTRPGSPDYRPTVDVFVPSYNEDAELLANTLAAAKNMDYPADRFTVWLLDDGGSVQKRNAANIVEAQAAQRRHEELKKLCEDLDVRYLTRERNVHAKAGNLNNGLAHSTGELVTVFDADHAPARDFLLETVGYFDEDPRLFLVQTPHFFVNPDPIERNLRTFETMPSENEMFYGIIQRGLDKWNGAFFCGSAAVLRREALQDSDGFSGVSITEDCETALALHSRGWNSVYVDKPLIAGLQPATFASFIGQRSRWAQGMMQILIFRQPLFKRGLSFTQRLCYMSSTLFWLFPFPRTIFLFAPLFYLFFDLQIFVASGGEFLAYTAAYMLVNLMMQNYLYGSFRWPWISELYEYVQTVHLLPAVVSVIFNPGKPTFKVTAKDESIAEARLSEISRPFFVIFALLLVAMAFAVWRIYSEPYKADVTLVVGGWNLLNLIFAGCALGVVSERGDKSASRRITVKRRCEVQLGGSDTWVPASIDNVSVHGLLINIFDSATNIEKGATAIVKVKPHSEGVPETMPLNVVRTVRGEGFVSIGCTFSPQRAVDHRLIADLIFANSEQWSEFQRVRRKKPGLIRGTAIFLAIALFQTQRGLYYLVRARRPAPKSAKPVGAVK

SEQ ID NO: 69 - protein, 315 aa, Streptococcus agalactiae
MIKKIEKDLISVIVPIYNVEDYLVECIESLIVQTYRNIEILLINDGSTDNCATIAKEFSERDCRVIYIEKSNGGLSEARNYGIYHSKGKYLTFVDSDDKVSSDYIANLYNAIQKHDSSIAIGGYLEFYERHNSIRNYEYLDKVIPVEEALLNMYDIKTYGSIFITAWGKLFHKSIFNDLEFALNKYHEDEFFNYKAYLKANSITYIDKPLYHYRIRVGSIMNNSDNVIIARKKLDVLSALDERIKLITSLRKYSVFLQKTEIFYVNQYFRTKKFLKQQSVMFKEDNYIDAYRMYGRLLRKVKLVDKLKLIKNRFF

SEQ ID NO: 70 - protein, 416 aa, Streptococcus pneumoniae
MYTFILMLLDFFQNHDFHFFMLFFVFILIRWAVIYFHAVRYKSYSCSVSDEKLFSSVIIPVVDEPLNLFESVLNRISRHKPSEIIVVINGPKNERLVKLCHDFNEKLENNMTPIQCYYTPVPGKRNAIRVGLEHVDSQSDITVLVDSDTVWTPRTLSELLKPFVCDKKIGGVTTRQKILDPERNLVTMFANLLEEIRAEGTMKAMSVTGKVGCLPGRTIAFRNIVERVYTKFIEETFMGFHKEVSDDRSLTNLTLKKGYKTVMQDTSVVYTDAPTSWKKFIRQQLRWAEGSQYNNLKMTPWMIRNAPLMFFIYFTDMILPMLLISFGVNIFLLKILNITTIVYTASWWEIILYVLLGMIFSFGGRNFKAMSRMKWYYVFLIPVFIIVLSIIMCPIRLLGLMRCSDDLGWGTRNLTE

SEQ ID NO: 71 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 72 - protein, 466 aa, Hordeum vulgare
MAQAESERMRVVMFPWLAHGHINPYLELAKRLIASASGDHHLDVVVHLVSTPANLAPLAHHQTDRLRLVELHLPSLPDLPPALHTTKGLPARLMPVLKRACDLAAPRFGALLDELCPDILVYDFIQPWAPLEAEARGVPAFHFATCGAAATAFFIHCLKTDRPPSAFPFESISLGGVDEDAKYTALVTVREDSTALVAERDRLPLSLERSSGFVAVKSSADIERKYMEYLSQLLGKEIIPTGPLLVDSGGSEEQRDGGRIMRWLDGEEPGSVVFVSFGSEYFMSEHQMAQMARGLELSGVPFLWVVRFPNAEDDARGAARSMPPGFEPELGLVVEGWAPQRRILSHPSCGAFLTHCGWSSVLESMAAGVPMVALPLHIDQPLNANLAVELGAAAARVKQERFGEFTAEEVARAVRAAVKGKEGEAARRRARELQEVVARNNGNDGQIATLLQRMARLCGKDQAVPN

SEQ ID NO: 73 - protein, 476 aa, Hordeum vulgare
MAEANDGGKMHVVMLPWLAFGHVLPFTEFAKRVARQGHRVTLLSAPRNTRRLIDIPPGLAGLIRVVHVPLPRVDGLPEHAEATIDLPSDHLRPCLRRAFDAAFERELSRLLQEEAKPDWVLVDYASYWAPTAAARHGVPCAFLSLFGAAALSFFGTPETLLGIGRHAKTEPAHLTVVPEYVPFPTTVAYRGYEARELFEPGMVPDDSGVSEGYRFAKTIEGCQLVGIRSSSEFEPEWLRLLGELYRKPVIPVGLFPPAPQDDVAGHEATLRWLDGQAPSSVVYAAFGSEVKLTGAQLQRIALGLEASGLPFIWAFRAPTSTETGAASGGLPEGFEERLAGRGVVCRGWVPQVKFLAHASVGGFLTHAGWNSIAEGLAHGVRLVLLPLVFEQGLNARNIVDKNIGVEVARDEQDGSFAAGDIAAALRRVMVEDEGEGFGAKVKELAKVFGDDEVNDQCVREFLMHLSDHSKKNQGQD

SEQ ID NO: 74 - protein, 453 aa, Coffea canephora
MAENHATFNVLMLPWLAHGHVSPYLELAMKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNTIFYRDYESVFVENLKKRDRDTYRVVNCMERSSKIILIKGFKEIEGKYFDYFSCLTGKKVVPVGPLVQDPVLDDEDCRIMQWLNKKEKGSTVFVSFGSEYFLSKEDMEEIAHGLELSNVDFIWVVRFPKGENIVIEETLPKGFFERVGERGLVVNGWAPQAKILTHPNVGGFVSHCGWNSVMESMKFGLPIVAMPMHLDQPINARLIEEVGAGVEVLRDSKGKLHRERMAETINKVTKEASGEPARKKARELQEKLELKGDEEIDDVVKELVQLCATKNKRNGLHCYN

SEQ ID NO: 75 - protein, 452 aa, Coffea eugenioides
MAENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAEPNFCNVLKSLKPDLLIYDLLQPWAPEAASAFNIPAVVFISSSATMTSFGLHFFKNPGTKYPYGNTIFYRDYESVFVENLKRRDRDTYRVVNCMERSSKIILIKGFKEIEGKYFDYFSCLTGKKVVPVGPLVQDPVLDDEDCRIMQWLNKKEKGSTVFVSFGSEYFLSKEDMEEIAHGLELSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVTKEASGESVRKKARELQEKLELKGDEEIDDVVKELVQLCATKNKRNGLHYN

SEQ ID NO: 76 - protein, 452 aa, Coffea eugenioides
MAENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNAIFYRDYESVFVENLTRRDRDTYRVINCMERSSKIILIKGFNEIEGKYFDYFSCLTGKKVVPVGPLVQDPVLDDEDCEIMQWLNKKEKVSTVFVSFGSEYFLSKKDMEEIAHGLELSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVMKEASGESVRKKARELQEKMDLKGDEEIDDVVKELVQLCATKNKRNGLHYY

SEQ ID NO: 77 - protein, 453 aa, Siraitia grosvenorii
MADAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 78 - protein, 474 aa, Oryza sativa
MAQAERERLRVLMFPWLAHGHINPYLELATRLTTTSSSQIDVVVHLVSTPVNLAAVAHRRTDRISLVELHLPELPGLPPALHTTKHLPPRLMPALKRACDLAAPAFGALLDELSPDVVLYDFIQPWAPLEAAARGVPAVHFSTCSAAATAFFLHFLDGGGGGGGRGAFPFEAISLGGAEEDARYTMLTCRDDGTALLPKGERLPLSFARSSEFVAVKTCVEIESKYMDYLSKLVGKEIIPCGPLLVDSGDVSAGSEADGVMRWLDGQEPGSVVLVSFGSEYFMTEKQLAEMARGLELSGAAFVWVVRFPQQSPDGDEDDHGAAAARAMPPGFAPARGLVVEGWAPQRRVLSHRSCGAFLTHCGWSSVMESMSAGVPMVALPLHIDQPVGANLAAELGVAARVRQERFGEFEAEEVARAVRAVMRGGEALRRRATELREVVARRDAECDEQIGALLHRMARLCGKGTGRAAQLGH

SEQ ID NO: 79 - protein, 443 aa, Panax ginseng
MADNQNGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 80 - protein, 499 aa, Stevia rebaudiana
MAHHHHHHVGTGSNDDDDKSPDPNWASTSELVFIPSPGAGHLPPTVELAKLLLHRDQRLSVTIIVMNLWLGPKHNTEARPCVPSLRFVDIPCDESTMALISPNTFISAFVEHHKPRVRDIVRGIIESDSVRLAGFVLDMFCMPMSDVANEFGVPSYNYFTSGAATLGLMFHLQWKRDHEGYDATELKNSDTELSVPSYVNPVPAKVLPEVVLDKEGGSKMFLDLAERIRESKGIIVNSCQAIERHALEYLSSNNNGIPPVFPVGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 81 - protein, 494 aa, Camelina sativa
MASEKTLQVHPPLHFVLFPFMAQGHMIPMVDIARLLAQRGATVTIVTTRYNAGRFENVLSRAVESGLPINIVHVKFPYEEVGLPKGKENIDSLDSMELMVPFFKAVNMLQDPVVKLMEEMESRPSCIISDLLLPYTSKIAKKFNIPKIVFHGISCFCLLCVHVLRRNLEILTNLKSDKEYFLVPSFPDRVEFTKPQVTVETNASGDWKEFLDEMVEAEDTSYGVIINTFEELEPAYVKDYKDARAGNVWSIGPVSLCNKAGVDKAERGNKATIDQDECLKWLDSKEEGSVLYVCLGSICNLPLVQLKELGLGLEESQRPFIWVIRGWEKYNELSEWMVESGFEERIRERGLLIRGWAPQVLILSHPSVGGFLTHCGWNSTVEGITSGVPLITWPLFGDQFCNQTLVVQVLKAGVSVGVEEVMKWGEEEKIGVLVDKEGVKKAVEDLMGESDDAKERTKRVKELGGLAHKAVEEGGSSHSNITLFLQDIRQVQSV

SEQ ID NO: 82 - protein, 478 aa, Glycyrrhiza uralensis
MADVAEEQPLKIYFIPYLAAGHMIPLCDIATLFASRGHHVTIITTPSNAQTLRESHHFRVQTIQFPSQEVGLPAGVQNLTAVTNLDDSYKIYHATMLLRKHIEDFVERDPPDCIVADFLFPWVDDVATKLHIPRLVFNGFTLFTICAMESHKAHPLPVDAASGSFVIPDFPHHVTINSTPPKRTKEFVDPLLTEAFKSHGFLINSFVELDGEECVEHYERITGGHKAWHLGPAFLVHRTAQDRGEKSVVSTQECLSWLDSKRDNSVLYICFGTICYFPDKQLYEIASAIEASGHEFIWVVPEKRGNADESEEEKEKWLPKGFEERNNGKKGMIIRGWAPQVAILGHPAVGGFLTHCGWNSTVEAVSAGVPMITWPVHSDQYFNEKLITQVRGIGVEVGAEEWIVTAFRETEKLVGRDRIERAVRRVMDGGDEAVQIRRRARELGEMARQAVQEGGSSHTNLTALINDLKRWRDSKQLN

SEQ ID NO: 83 - protein, 491 aa, Glycyrrhiza uralensis
MAVFQANQPHFVLFPLMAQGHIIPMIDIARLLAQRGAIVTIFTTPKNASRFTSVLSRAVSSGLQIRLVHLHFPSKEAGLPEGCENLDMVASHDMICNIFQAIRMLQKQAEELFETLTPKPSCIISDFCIPWTTQVAEKHHIPRISFHGFSCFCLHCMLKIHTSKVLEGITSESEYFTVPGIPDQIQVTKQQVPGPMIDEMKEFGEQMRDAEIRSYGVIINTFEELEKAYVNDYKKERNGKVWCIGPVSLCNKDGLDKAQRGNKASISEHHCLEWLDLQQPNSVIYVCLGSLCNLTPPQLMELALGLEATKRPFIWVIREGNKFEELEKWISEEGFEERIKGRGLIIRGWAPQVLILSHPSIGGFLTHCGWNSTLEGVTAGVPMVTWPLFADQFLNEKLVTQVLRIGVSLGVDVPLKWGEEEKVGVQVKKEGIEKAICMVMDEGEESKERRERAKELSEMAKRAVEKDGSSHLNMTMLIQDIMQQSSSKVET

SEQ ID NO: 84 - protein, 483 aa, Stevia rebaudiana
MDQMAKIDEKKPHVVFIPFPAQSHIKCMLKLARILHQKGLYITFINTDTNHERLVASGGTQWLENAPGFWFKTVPDGFGSAKDDGVKPTDALRELMDYLKTNFFDLFLDLVLKLEVPATCIICDGCMTFANTIRAAEKLNIPVILFWTMAACGFMAFYQAKVLKEKEIVPVKDETYLTNGYLDMEIDWIPGMKRIRLRDLPEFILATKQNYFAFEFLFETAQLADKVSHMIIHTFEELEASLVSEIKSIFPNVYTIGPLQLLLNKITQKETNNDSYSLWKEEPECVEWLNSKEPNSVVYVNFGSLAVMSLQDLVEFGWGLVNSNHYFLWIIRANLIDGKPAVMPQELKEAMNEKGFVGSWCSQEEVLNHPAVGGFLTHCGWGSIIESLSAGVPMLGWPSIGDQRANCRQMCKEWEVGMEIGKNVKRDEVEKLVRMLMEGLEGERMRKKALEWKKSATLATCCNGSSSLDVEKLANEIKKLSRN

SEQ ID NO: 85 - protein, 6 aa, Artificial Sequence
  Synthetic Sequence
GGSGGS

SEQ ID NO: 86 - protein, 7 aa, Artificial Sequence
  Synthetic Sequence
GGSGGSG

SEQ ID NO: 87 - protein, 8 aa, Artificial Sequence
  Synthetic Sequence
GGSGGSGG

SEQ ID NO: 88 - protein, 9 aa, Artificial Sequence
  Synthetic Sequence
GGSGGSGGS

SEQ ID NO: 89 - protein, 10 aa, Artificial Sequence
  Synthetic Sequence
GGSGGSGGSG

SEQ ID NO: 90 - protein, 11 aa, Artificial Sequence
  Synthetic Sequence
GGSGGSGGSGG

SEQ ID NO: 91 - protein, 12 aa, Artificial Sequence
  Synthetic Sequence
GGSGGSGGSGGS

SEQ ID NO: 92 - protein, 546 aa, Escherichia coli
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 93 - protein, 302 aa, Escherichia coli
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 94 - protein, 219 aa, Escherichia coli [sequence omitted]

SEQ ID NO: 95 - protein, 489 aa, Bifidobacterium bifidum
MAFAEDLKRTEKMTVDDVFEQSAQKMREQGMSEIAISQFRHAYHVWASEKESAWIREDTVEPLHGVRSFHDVYKTIDHDKAVHAFAKTAFLKLNGGLGTSMGLQCAKSLLPVRRHKARQMRFLDIILGQVLTARTRLNVPLPVTFMNSFRTSDDTMKALRHQRKFKQTDIPLEIIQHQEPKIDAATGAPASWPANPDLEWCPPGHGDLFSTLRESGLLDTLLEHGFEYLFISNSDNLGARPSRTLAQYFEDTGAPFMVEVANRTYADRKGGHIVRDTATGRLILREMSQVHPDDKDAAQDIAKHPYFNTNNIWVRIDVLRDMLAEHDGVLPLPVIINNKTVDPIDPQSPAVVQLETAMGAAIGLFEGAICVQVDRMRFLPVKTTNDLFIMRSDRFHLTDSYEMEDGNYIFPNVDLDPRYYKNIEDFNERFPYNVPSLAAANSVSIKGDWTFGRDVIMFADARLEDRNEPSYVPNGEYVGPMGIEPGDWV

SEQ ID NO: 96 - protein, 214 aa, Escherichia coli
MRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILG

SEQ ID NO: 97 - protein, 143 aa, Escherichia coli
MAIERTFSIIKPNAVAKNVIGNIFARFEAAGFKIVGTKMLHLTVEQARGFYAEHDGKPFFDGLVEFMTSGPIVVSVLEGENAVQRHRDLLGATNPANALAGTLRADYADSLTENGTHGSDSVESAAREIAYFFGEGEVCPRTR

SEQ ID NO: 98 - protein, 227 aa, Escherichia coli
MTAIAPVITIDGPSGAGKGTLCKAMAEALQWHLLDSGAIYRVLALAALHHHVDVASEDALVPLASHLDVRFVSTNGNLEVILEGEDVSGEIRTQEVANAASQVAAFPRVREALLRRQRAFRELPGLIADGRDMGTVVFPDAPVKIFLDASSEERAHRRMLQLQEKGFSVNFERLLAEIKERDDRDRNRAVAPLVPAADALVLDSTTLSIEQVIEKALQYARQKLALA

SEQ ID NO: 99 - protein, 474 aa, Arabidopsis thaliana
MANNNSNSPTGPHFLFVTFPAQGHINPSLELAKRLAGTISGARVTFAASISAYNRRMFSTENVPETLIFATYSDGHDDGFKSSAYSDKSRQDATGNFMSEMRRRGKETLTELIEDNRKQNRPFTCVVYTILLTWVAELAREFHLPSALLWVQPVTVFSIFYHYFNGYEDAISEMANTPSSSIKLPSLPLLTVRDIPSFIVSSNVYAFLLPAFREQIDSLKEEINPKILINTFQELEPEAMSSVPDNFKIVPVGPLLTLRTDFSSRGEYIEWLDTKADSSVLYVSFGTLAVLSKKQLVELCKALIQSRRPFLWVITDKSYRNKEDEQEKEEDCISSFREELDEIGMVVSWCDQFRVLNHRSIGCFVTHCGWNSTLESLVSGVPVVAFPQWNDQMMNAKLLEDCWKTGVRVMEKKEEEGVVVVDSEEIRRCIEEVMEDKAEEFRGNATRWKDLAAEAVREGGSSFNHLKAFVDEHM